(19) United States
(12) Patent Application Publication
(10) Pub. No.: US 2015/0025006 A1
(43) Pub. Date: Jan. 22, 2015

(54) Pharmaceutical Combinations Including Anti-Inflammatory and Antioxidant Conjugates Useful for Treating Metabolic Disorders

(71) Applicants: Julio Cesar Castro Palomino Laria, Mataro (ES); Luc Marti Clauzel, Barcelona (ES); Antonio Zorzano Olarte, Barcelona (ES); Silvia Garcia Vicente, Sant Feliu de Llobregat (ES); Alec Mian, Barcelona (ES)

(72) Inventors: Julio Cesar Castro Palomino Laria, Mataro (ES); Luc Marti Clauzel, Barcelona (ES); Antonio Zorzano Olarte, Barcelona (ES); Silvia Garcia Vicente, Sant Feliu de Llobregat (ES); Alec Mian, Barcelona (ES)

(73) Assignee: Genmedica Therapeutics SL, Barcelona (ES)

(21) Appl. No.: 14/344,529

(22) PCT Filed: Sep. 14, 2012
(86) PCT No.: PCT/EP2012/068179

§ 371 Date: Oct. 6, 2014

Related U.S. Application Data
(60) Provisional application No. 61/535,865, filed on Sep. 16, 2011.

(51) Int. Cl. A61K 31/265; A61K 38/23; A61K 38/22; A61K 31/155; A61K 38/26

(52) U.S. Cl. CPC A61K 31/265; A61K 31/155; A61K 38/26; A61K 38/2207; A61K 38/23
USPC 514/6.7; 514/506; 514/12.3; 514/11.9; 514/7.2; 514/11.7

(57) ABSTRACT

One aspect of the present invention is a pharmaceutical combination comprising (a) an anti-inflammatory agent/anti-oxidant agent conjugate; and (b) an insulin secretogogue, an insulin sensitizer, an alpha-glucosidase inhibitor, a peptide analog, or a combination thereof. Another aspect of the invention relates to methods of treating metabolic disorders with such conjugates.

### CROSS-REFERENCE TO RELATED APPLICATIONS

This application claims the benefit of priority to U.S. Provisional Patent Application Ser. No. 61/535,865, filed Sep. 16, 2011, which is hereby incorporated herein by reference in its entirety.

### BACKGROUND

Oxidative stress and inflammation are implicated in the pathogenesis of metabolic diseases, diabetes, obesity, dyslipidemia and their associated cardiovascular complications. For example, oxidative stress is a common pathogenic factor leading to insulin resistance, β-cell dysfunction, impaired glucose tolerance, and type 2 diabetes mellitus. With regard to inflammation, clinical studies suggest that acute hyperglycemia results in elevated levels of circulating inflammatory cytokines such as TNFα, IL6, and IL18.

During hyperglycemia and/or hyperlipidemia, mitochondria generate cellular energy through TCA cycle activity and the associated electron transport chain of the inner mitochondrial membrane. However, while mitochondria generate elevated ATP production, mitochondria can also generate significant reactive oxygen species (ROS) and reactive nitrogen species (RNS). Cells are equipped with several antioxidant enzymes to neutralize ROS and RNS. For example, superoxide anions are enzymatically converted to hydrogen peroxide by a manganese superoxide dismutase (MnSOD) within mitochondria. Hydrogen peroxide can then be rapidly removed by the mitochondrial enzyme glutathione (GSH) peroxidase. A further antioxidant enzyme, catalase, is the hydrogen peroxide detoxifying enzyme founded exclusively in peroxisomes. Glutathione (GSH) is probably the most important defense with which the cell is equipped, for scavenging ROS generated by mitochondria metabolism and excess free radicals produced secondary to hyperglycemia and hyperlipidemia.

However, while cells have a number of available anti-oxidant mechanisms, damage most likely occurs when the ROS is excessive and/or anti-oxidant pathways are overwhelmed as is frequently the case in diabetes. In diabetic patients, the levels of antioxidant enzymes responsible for scavenging free radicals are diminished. Glutathione pools become depleted in diabetic patients following frequent and severe hyperglycemic episodes. It is now widely accepted that overproduction of reactive oxygen species (ROS) contributes to cell and tissue dysfunction and damage caused by glucolipotoxicity in diabetes, insulin resistance, and obesity.

In particular, compared to several other cells of the body, pancreatic β-cells have relatively low levels of free radical detoxification and redox regulating enzymes such as superoxide dismutase, glutathione peroxidase, catalase and thioredoxin. The consequence of limited scavenging systems is that ROS concentration in β-cells may increase rapidly, damaging the β-cells. Thus, under hyperglycemic conditions, the production of ROS, and subsequent oxidative stress, contributes to β-cell deterioration observed in type 2 diabetes.

ROS is also considered a strong stimulus for the release of cytokines and increased superoxide can promote inflammation through NF-kB activation. Thus the role of oxidative stress and associated activation of NF-kB leading to chronic inflammation and insulin resistance is essential in the processes implicated in the pathogenesis of diabetes and its progression. Administration of glutathione, a powerful antioxidant, completely suppresses cytokine elevation, providing further support that an oxidative stress mechanism mediates the inflammatory effects of hyperglycemia in humans.

Salicylates, or aspirin-like drugs, are some of the most commonly used anti-inflammatory agents. For more than two decades, the anti-inflammatory properties of aspirin have been almost exclusively attributed to blocking prostaglandin synthesis via inhibition of cyclo-oxygenase activity. Recently, aspirin and sodium salicylate have been found to inhibit the activation of the transcription factor NF-kB. High doses of salicylate are thought to inhibit NF-kB and its upstream activator, the IKB kinase β (IKKβ).

Also, high doses of salicylic acid lower blood glucose levels. Recent studies report that diabetic animals given salicylates or salsalate showed a decrease in IKKβ activity, accompanied by improvement in insulin sensitivity. High doses of Salicylate (120 mg/kg/day) administered by subcutaneous infusion in Zucker fa/fa rats or ob/ob mice for 3-4 weeks exhibited anti-diabetic effects, reduction in fasting blood glucose, and glucose tolerance improvement. Beneficial effects of high doses of salicylic acid have been recently reported in human diabetic patients treated with 4.5 g/day of salsalate. However, at this high dose, side effects, such as tinnitus, are enhanced by 66% and the long term risk of gastric bleeding and ulceration is also increased.

Thus, there remains a need in the art for compounds for treating metabolic disorders by way of ameliorating the inflammatory and oxidative processes associated with such disorders, particularly diabetes.

### SUMMARY OF THE INVENTION

The present invention relates to pharmaceutical combinations including (a) an anti-inflammatory agent/anti-oxidant agent conjugate, and (b) an insulin secretagogue, an insulin sensitizer, a peptide analog, or a combination thereof. The pharmaceutical combinations of the present invention are useful for treating atherosclerosis, neuropathy, nephropathy, retinopathy, inflammatory disorders, Chronic Obstructive Pulmonary Disease (COPD), cardiovascular diseases, and metabolic disorders, such as any form of diabetes mellitus including type I and type II diabetes and Latent Autoimmune Diabetes of Adulthood (LADA), metabolic syndrome, hyperglycemia, and insulin sensitivity. The combinations are also useful for reducing advanced glycated end products (AGEs), ROS, lipid peroxidation, tissue and plasma TNFα and IL6 levels, and for delaying or preventing cardiovascular complications associated with atherosclerosis. Also, the pharmaceutical combinations of the present invention are useful for protecting pancreatic β-cells, preventing their impairment or failure and subsequent lower insulin secretion.

The present invention provides combinations, as described herein. In another aspect, the present invention provides pharmaceutical compositions including a pharmaceutical combination as described herein and at least one pharmaceutically acceptable carrier. The pharmaceutical combinations and the pharmaceutical compositions including these pharmaceutical combinations are useful for treating atherosclerosis, neuropathy, nephropathy, retinopathy, inflammatory disorders, Chronic Obstructive Pulmonary Disease (COPD), cardiovascular diseases, metabolic disorders, type I diabetes mellitus, type II diabetes mellitus, Latent Autoimmune Diabetes of Adulthood (LADA), metabolic syndrome, dyslipidemia, hyperglycemia, or insulin resistance. The pharmaceutical combinations and pharmaceutical compositions of the present invention are useful for protecting pancreatic β-cells, preventing their impairment or failure and subsequent lower insulin secretion. Also, the compounds and pharmaceutical compositions of the present invention are also useful for reducing free fatty acids (FFA), triglycerides, advanced glycated end products (AGEs), ROS, lipid peroxidation, tissue and plasma TNFα and IL6 levels, or for delaying or preventing cardiovascular complications associated with atherosclerosis.

In another aspect, the present invention provides methods for treating atherosclerosis, neuropathy, nephropathy, retinopathy, inflammatory disorders, Chronic Obstructive Pulmonary Disease (COPD), cardiovascular diseases, metabolic disorders, type I diabetes mellitus, type II diabetes mellitus, Latent Autoimmune Diabetes of Adulthood (LADA), metabolic syndrome, dyslipidemia, hyperglycemia, or insulin resistance in a mammal or human patient including administering to the mammal or human patient in need of such treatment a therapeutically effective amount of a pharmaceutical combination of the disclosure or a pharmaceutical composition including a pharmaceutical combination of the disclosure. The present invention also provides methods for reducing free fatty acids (FFA), triglycerides, advanced glycated end products (AGEs), ROS, lipid peroxidation, tissue and plasma TNFα and IL6 levels, or for delaying or preventing cardiovascular complications associated with atherosclerosis in a mammal or human patient comprising administering to the mammal or human patient in need of such treatment a therapeutically effective amount of a pharmaceutical combination of the disclosure or a pharmaceutical composition including a pharmaceutical combination of the disclosure. Also, the present invention provides methods for protecting pancreatic β-cells, preventing their impairment or failure and subsequent lower insulin secretion in a mammal or human patient comprising administering to the mammal or human patient in need of such treatment a therapeutically effective amount of a pharmaceutical combination of the disclosure or a pharmaceutical composition including a pharmaceutical combination of the disclosure.

In another aspect, the present invention provides uses for pharmaceutical combinations of the disclosure, or pharmaceutical compositions including a pharmaceutical combination of the disclosure, for preparing, or for the manufacture of, a medicament for treating atherosclerosis, neuropathy, nephropathy, retinopathy, inflammatory disorders, Chronic Obstructive Pulmonary Disease (COPD), cardiovascular diseases, metabolic disorders, type I diabetes mellitus, type II diabetes mellitus, Latent Autoimmune Diabetes of Adulthood (LADA), metabolic syndrome, dyslipidemia, hyperglycemia, or insulin resistance in a mammal or human patient. The present invention also provides uses for pharmaceutical combinations of the disclosure, or pharmaceutical compositions including a pharmaceutical combination of the disclosure, for preparing, or for the manufacture of, a medicament for reducing free fatty acids (FFA), triglycerides, advanced glycated end products (AGEs), ROS, lipid peroxidation, tissue and plasma TNFα and IL6 levels, or for delaying or preventing cardiovascular complications associated with atherosclerosis in a mammal or human patient. The present invention also provides uses for pharmaceutical combinations of the disclosure, or pharmaceutical compositions including a pharmaceutical combination of the disclosure, for preparing, or for the manufacture of, a medicament for protecting pancreatic β-cells, preventing their impairment or failure and subsequent lower insulin secretion, in a mammal or human patient.

Specific embodiments of the present invention will become evident from the following more detailed description of certain preferred embodiments and the claims.

### BRIEF DESCRIPTION OF THE DRAWINGS

The results set forth herein, and the properties and characteristics of the conjugates provided by the invention, can be advantageously understood with regard to the drawings. In each of the drawings comprising bar graphs, the legends identify the bars in left-to-right order.

FIG. 1 shows the non-fasting glycemia levels, insulin tolerance and pancreatic insulin content in db/db mice after treatment with the compounds according to certain embodiments of the invention identified in the figure legend.

FIG. 2 shows the fasting glycemia levels in db/db mice after treatment with the compounds according to certain embodiments of the invention identified in the figure legend.

FIG. 3 shows the level of non-esterified fatty acids (NEFA) in db/db mice after treatment with the compounds according to certain embodiments of the invention identified in the figure legend.

FIG. 4 illustrates the pancreas insulin level after treatment with the compounds according to certain embodiments of the invention identified in the figure legend.

### DETAILED DESCRIPTION

The present invention provides combination and methods for treating atherosclerosis, neuropathy, nephropathy, retinopathy, inflammatory disorders, Chronic Obstructive Pulmonary Disease (COPD), cardiovascular diseases, and metabolic disorders in a mammal or patient comprising administering to the mammal or patient in need of such treatment a therapeutically effective amount of a combination comprising:

(a) an anti-inflammatory agent/anti-oxidant agent conjugate; and

(b) an insulin secretagogue, an insulin sensitizer, a peptide analog, or a combination thereof.

In one embodiment (embodiment 1), the combination of the disclosure comprises:

(a) the conjugate as described above; and

(b) the insulin secretagogue.

In embodiment 2, the combination according to embodiment 1 is wherein the insulin secretagogue is a sulfonylurea or meglitinide. In embodiment 3, the insulin secretagogue is sulfonylurea. Embodiment 4 includes the combination of embodiments 1-3 wherein the insulin secretagogue is selected from the group consisting of: tolbutamide (Orinase), acetohexamide (Dymelor), tolazamide (Tolinase), chlorpropamide (Diabinese), glipizide (Glucotrol), carbutamide (Glucidoral), glyburide (Diabeta, Micronase, Glynase), glimepiride (Amaryl), and gliclazide (Diamicron). Embodiment 5 includes the combination of embodiments 1-2 wherein the insulin secretagogue is meglitinide. In embodiment 6, meglitinide is repaglinide (Prandin) or nateglinide (Starlix).

In one embodiment (embodiment 7), the combination of the disclosure comprises:

(a) the conjugate as described above; and

(b) the insulin sensitizer.

In embodiment 8, the combination according to embodiment 8 is wherein the insulin sensitizer is a biguanide or thiazolidinedione. In embodiment 10, the insulin sensitizer is biguanide. Embodiment 9 includes the combination of embodiments 7-9 wherein the insulin sensitizer is selected from the group consisting of: metformin (Glucophage), phenfonnin (DBI), and bufonnin. Embodiment 10 includes the combination wherein the insulin sensitizer is metformin. In embodiment 11, the combination of embodiments 7-8 comprises the insulin secretagogue, which is meglitinide. In embodiment 12, the insulin sensitizer is thiazolidinedione. Embodiment 13 provides the combinations wherein the thiazolidinedione is rosiglitazone (Avandia), pioglitazone (Actos), or troglitazone (Rezulin).

Embodiment 14 provides the combination of the disclosure comprising:

(a) the conjugate as described above; and

(b) the peptide analog.

In embodiment 15, the combination according to embodiment 14 is where the peptide analog is selected from the group consisting of: glucagon-like peptide (GLP) analogs and agonists, gastric inhibitory peptide analogs, and amylin analogues.

In embodiment 16, the combination of embodiments 14-15 comprises the peptide analog which is glucagon-like peptide (GLP) analog or agonist. In embodiment 17, glucagon-like peptide (GLP) analog is selected from the group consisting of: exenatide (Exendin-4, Byetta), liraglutide (Victoza), Albiglutide, and Taspoglutide.

Embodiment 18 provides combinations wherein the peptide analog is exenatide (Exendin-4, Byetta).

In embodiment 19, the combination of embodiments 14-15 comprises the peptide analog which is gastric inhibitory peptide analogs.

Embodiment 20 provides the combination of the disclosure comprising:

(a) the conjugate as described above; and

(b) a combination of an insulin secretagogue, an insulin sensitizer, an alpha-glucosidase inhibitor, undo peptide analog.

Embodiment 21 includes the combination of embodiments 1-20, wherein the conjugate is a preferred, specifically-named, or example conjugate as described in the above-referenced publications.

The anti-inflammatory agent/anti-oxidant agent conjugates useful in certain aspects of the present invention are disclosed in International Patent Application No. PCT/EP2010/053418, filed on Mar. 16, 2010 (published as WO 2010/106082 on Sep. 23, 2010); U.S. patent application Ser. No. 13/235,031, filed Sep. 16, 2011; and U.S. Provisional Patent Application Ser. No. 61/535,803, filed Sep. 16, 2011; each of which is hereby incorporated herein by reference in its entirety.

In certain embodiments, the anti-inflammatory agent/anti-oxidant agent conjugate is selected from
(R)-2-acetamido-3-(2-hydroxybenzoylthio)propanoic acid;
(R)-methyl 2-acetamido-3-(2-hydroxybenzoylthio)propanoate;
(R)-ethyl 2-acetamido-3-(2-hydroxybenzoylthio)propanoate;
(R)-2-acetamido-3-(2-acetoxybenzoylthio)propanoic acid;
(R)-methyl 2-acetamido-3-(2-acetoxybenzoylthio)propanoate;
(R)-ethyl 2-acetamido-3-(2-acetoxybenzoylthio)propanoate;
(R)-2-acetamido-3-(2-hydroxy-4-(trifluoromethyl)benzoylthio)propanoic acid;
(R)-methyl 2-acetamido-3-(2-hydroxy-4-(trifluoromethyl)benzoylthio)propanoate;
(R)-ethyl 2-acetamido-3-(2-hydroxy-4-(trifluoromethyl)benzoylthio)propanoate;
(R)-2-acetamido-3-(2-acetoxy-4-(trifluoromethyl)benzoylthio)propanoic acid;
(R)-methyl 2-acetamido-3-(2-acetoxy-4-(trifluoromethyl)benzoylthio)propanoate;
(R)-ethyl 2-acetamido-3-(2-acetoxy-4-(trifluoromethyl)benzoylthio)propanoate;
(R)-2-acetamido-3-(2-acetoxy-4-(trifluoromethyl)benzoylthio)propanoic acid;
(R)-methyl 2-acetamido-3-(2-acetoxy-4-(trifluoromethyl)benzoylthio)propanoate;
(R)-ethyl 2-acetamido-3-(2-acetoxy-4-(trifluoromethyl)benzoylthio)propanoate;
(R)-2-acetamido-3-(2′,4′-difluoro-4-hydroxybiphenylcarbonylthio)propanoic acid (GMC-252);
(R)-methyl 2-acetamido-3-(2′,4′-difluoro-4-hydroxybiphenylcarbonylthio)propanoate;
(R)-ethyl 2-acetamido-3-(2′,4′-difluoro-4-hydroxybiphenylcarbonylthio)propanoate;
(R)-2-acetamido-3-(4-acetoxy-2′,4′-difluorobiphenylcarbonylthio)propanoic acid;
(R)-methyl 2-acetamido-3-(4-acetoxy-2′,4′-difluorobiphenylcarbonylthio)propanoate;
(R)-ethyl 2-acetamido-3-(4-acetoxy-2′,4′-difluorobiphenylcarbonylthio)propanoate;
methyl 2-(5-((R)-1,2-dithiolan-3-yl)pentanoyloxy)benzoate;
tert-butyl 2-(5-((R)-1,2-dithiolan-3-yl)pentanoyloxy)benzoate;
benzyl 2-(5-((R)-1,2-dithiolan-3-yl)pentanoyloxy)benzoate;
(R)-2-acetamido-3-((2′,4′-difluoro-3-(methoxycarbonyl)biphenyl-4-yloxy) carbonylthio)propanoic acid;
(R)-2-acetamido-3-((2-(methoxycarbonyl)phenoxy)carbonylthio)propanoic acid;
(R)-2-acetamido-3-((2′,4′-difluoro-3-(benzyloxycarbonyl)biphenyl-4-yloxy)carbonylthio)propanoic acid;
(R)-2-acetamido-3-((2-(benzyloxycarbonyl)phenoxy)carbonylthio)propanoic acid;
(+/−)-2-acetamido-4-((2′,4′-difluoro-3-(methoxycarbonyl)biphenyl-4-yloxy)carbonylthio)butanoic acid (GMC-300);
(+/−)-2-acetamido-4-((2-(methoxycarbonyl)phenoxy)carbonylthio) butanoic acid
(R)-2-acetamido-3-((2′,4′-difluoro-3-(ethoxycarbonyl)biphenyl-4-yloxy)carbonylthio)propanoic acid;
(R)-2-acetamido-3-((2′,4′-difluoro-3-(propoxycarbonyl)biphenyl-4-yloxy) carbonylthio)propanoic acid (GMC-316);
(R)-2-acetamido-3-((2′,4′-difluoro-3-(isopropoxycarbonyl)biphenyl-4-yloxy) carbonylthio)propanoic acid;
(R)-2-acetamido-3-((2-(ethoxycarbonyl)phenoxy)carbonylthio)propanoic acid;
(R)-2-acetamido-3-((2-(propoxycarbonyl)phenoxy)carbonylthio)propanoic acid;
(R)-2-acetamido-3-((2-(isopropoxycarbonyl)phenoxy)carbonylthio)propanoic acid;
(R)-2-acetamido-3-((2-(tert-butoxycarbonyl)phenoxy)carbonylthio)propanoic acid;
(R)-2-acetamido-3-((3-(tert-butoxycarbonyl)-2′,4′-difluorobiphenyl-4-yloxy)carbonylthio)propanoic acid;
(R)-benzyl 4-((2-acetamido-3-methoxy-3-oxopropylthio)carbonyloxy)-2′,4′-difluorobiphenyl-3-carboxylate;
(R)-tert-butyl 4-((2-acetamido-3-methoxy-3-oxopropylthio)carbonyloxy)-2′,4′-difluorobiphenyl-3-carboxylate;
(R)-2-acetamido-3-((2′,4′-difluoro-3-((4-methoxybenzyloxy)carbonyl) biphenyl-4-yloxy)carbonylthio)propanoic acid;
2-(2-((2′,4′-difluoro-3-(methoxycarbonyl)biphenyl-4-yloxy)carbonylthio) propanamido)acetic acid;
(R)-2-acetamido-3-((2-(benzyloxycarbonyl)-5-(trifluoromethyl)phenoxy) carbonylthio)propanoic acid, and
(S)-2-acetamido-4-(2′,4′-difluoro-4-hydroxybiphenylcarbonylthio)butanoic acid (GMC-299),
   and pharmaceutically acceptable salts (e.g., lysine salts) thereof

In certain embodiments, the anti-inflammatory agent/anti-oxidant agent conjugate is (R)-2-acetamido-3-(2′,4′-difluoro-4-hydroxybiphenylcarbonylthio)propanoic acid (GMC-252), or a pharmaceutically acceptable salt thereof (e.g., a lysine salt).

In certain embodiments, the anti-inflammatory agent/anti-oxidant agent conjugate is (R)-2-acetamido-3-((2′,4′-difluoro-3-(propoxycarbonyl)biphenyl-4-yloxy) carbonylthio)propanoic acid (GMC-316), or a pharmaceutically acceptable salt thereof (e.g., a lysine salt).

In certain embodiments, the anti-inflammatory agent/anti-oxidant agent conjugate is (S)-2-acetamido-4-(2′,4′-difluoro-4-hydroxybiphenylcarbonylthio)butanoic acid (GMC-299), or a pharmaceutically acceptable salt thereof (e.g., a lysine salt).

In certain embodiments, the anti-inflammatory agent/anti-oxidant agent conjugate is (+/−)-2-acetamido-4((2′,4′-difluoro-3-(methoxycarbonyl)biphenyl-4-yloxy)carbonylthio)butanoic acid (GMC-300), or a pharmaceutically acceptable salt thereof (e.g., a lysine salt).

In certain embodiments, the anti-inflammatory agent/anti-oxidant agent conjugate is a compound of Formula (A-I)

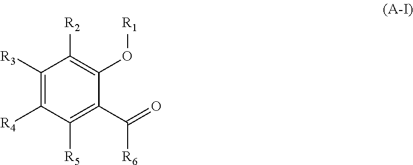

or a pharmaceutically acceptable salt thereof, wherein

R₁ is hydrogen, (C₁-C₆)alkylcarbonyl, or A;

R₂, R₃, R₄, and R₅ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁Z₂, or (NZ₁Z₂)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, (NZ₃Z₄)carbonyl;

Z₁, Z₂, Z₃, and Z₄ are independently hydrogen, (C₁-C₆)alkyl, or (C₁-C₆)alkylcarbonyl;

R₆ is hydroxy, —NZ₅Z₆,

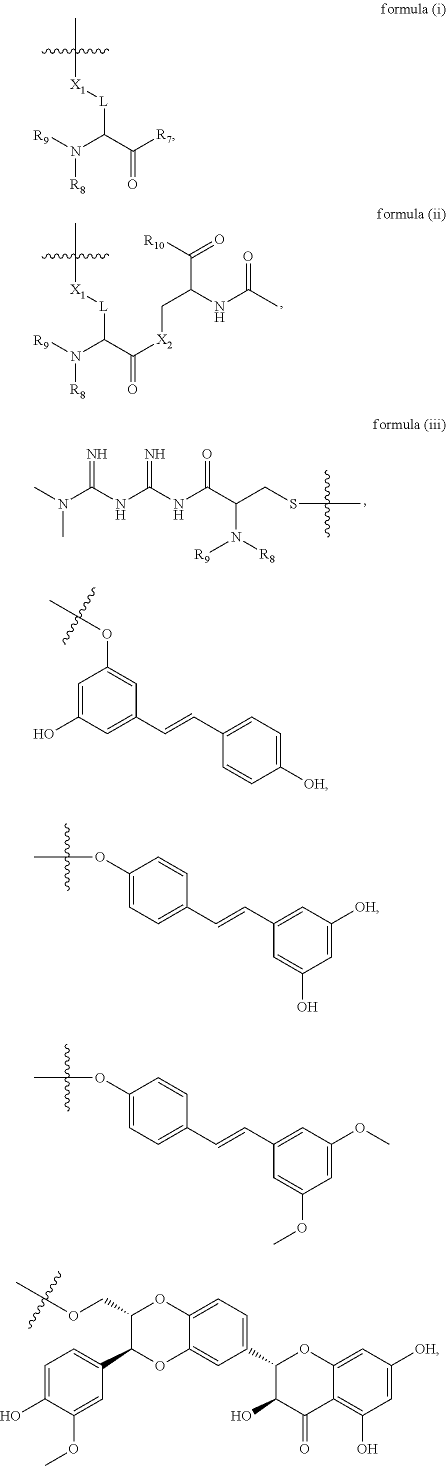

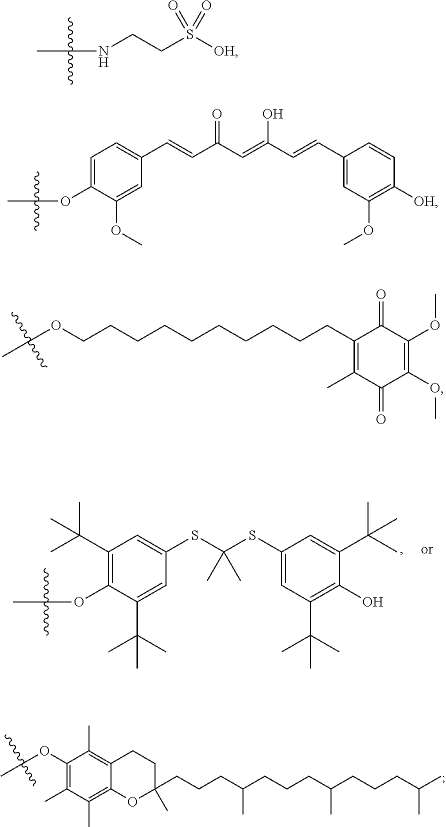 [SMILES: *NCCS(=O)(=O)O ; *Oc1ccc(/C=C/C(=O)/C=C(O)/C=C/c2ccc(O)c(OC)c2)cc1OC ; *OCCCCCCCCCCC1=C(C)C(=O)C(OC)=C(OC)C1=O ; *Oc1c(C(C)(C)C)cc(SC(C)(C)Sc2cc(C(C)(C)C)c(O)c(C(C)(C)C)c2)cc1C(C)(C)C ; *Oc1c(C)c(C)c2c(c1C)CCC(C)(CCCC(C)CCCC(C)CCCC(C)C)O2]

provided that when R₆ is hydroxy, then R₁ is A;

Z₅ and Z₆ are independently hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₃, or (NZ₇Z₈)carbonyl;

Z₇ and Z₈ are independently hydrogen, (C₁-C₆)alkyl, or (C₁-C₆)alkylcarbonyl;

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkyl, (C₁-C₆)alkylthio, hydroxy, or —NZ₉Z₁₀;

Z₉ and Z₁₀ are independently hydrogen, (C₁-C₆)alkyl, or (C₁-C₆)alkylcarbonyl;

X₁ and X₂ are independently O or S;

L is (C₁-C₆)alkylene;

A is

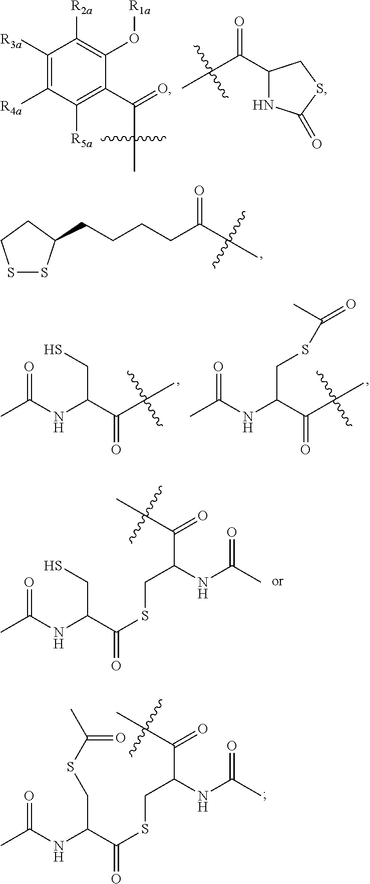

R₁ₐ is hydrogen, (C₁-C₆)alkylcarbonyl, or B;

R₂ₐ, R₃ₐ, R₄ₐ, and R₅ₐ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁ₐZ₂ₐ, or (NZ₁ₐZ₂ₐ)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkyl carbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃ₐZ₄ₐ, or (NZ₃ₐZ₄ₐ)carbonyl;

Z₁ₐ, Z₂ₐ, Z₃ₐ, and Z₄ₐ are independently hydrogen, (C₁-C₆)alkyl, or (C₁-C₆)alkylcarbonyl;

B is

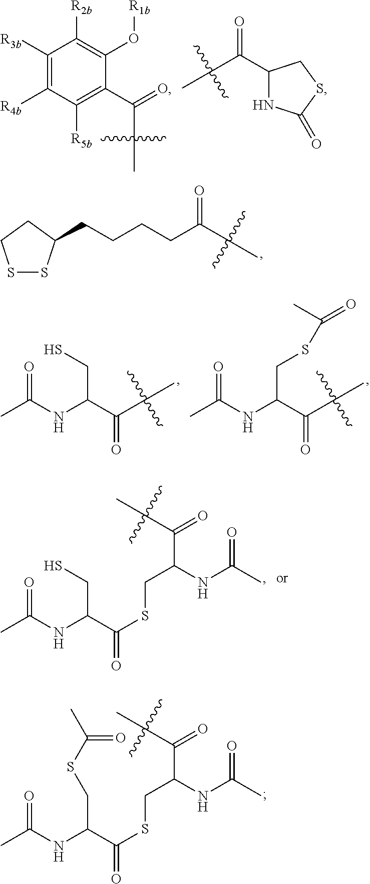

R_{1b} is hydrogen, (C₁-C₆)alkylcarbonyl, or C;

R_{2b}, R_{3b}, R_{4b}, and R_{5b} are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{1b}Z_{2b}, or (NZ_{1b}Z_{2b})carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{3b}Z_{4b}, or (NZ_{3b}Z_{4b})carbonyl;

Z_{1b}, Z_{2b}, Z_{3b}, and Z_{4b} are independently hydrogen, (C₁-C₆)alkyl, or (C₁-C₆)alkylcarbonyl; and

C is

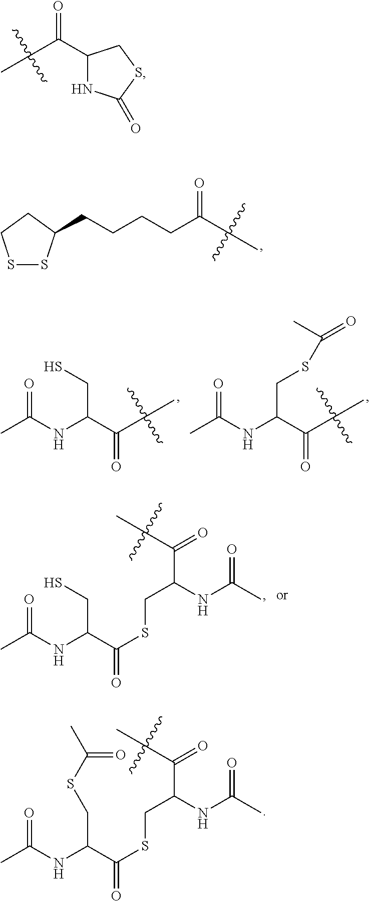

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, halo(C₁-C₆)alkyl, halogen, or phenyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, or (N₃Z₄)carbonyl; R₆ is formula (i); R₇ is (C₁-C₆)alkoxy, (C₁-C₆)alkyl, (C₁-C₆)alkylthio, hydroxy, or —NZ₉Z₁₀; R₈ is hydrogen or (C₁-C₆)alkyl; R₉ is (C₁-C₆)alkylcarbonyl; X₁ is O or S; L is (C₁-C₆)alkylene; and Z₃, Z₄, Z₉, and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen or phenyl, wherein the phenyl is optionally substituted with 1 or 2 groups that are independently halo(C₁-C₆)alkoxy, halo(C₁-C₆)alkyl, or halogen; R₆ is formula (i); R₇ is (C₁-C₆)alkoxy or hydroxy; R₈ is hydrogen or (C₁-C₆)alkyl; R₉ is (C₁-C₆)alkylcarbonyl; X₁ is O or S; and L is (C₁-C₆)alkylene.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen or phenyl, wherein the phenyl is optionally substituted with 1 or 2 halogen groups; R₆ is formula (i); R₇ is ethoxy, methoxy, or hydroxy; R₈ is hydrogen or methyl; R₉ is acetyl; X₁ is O or S; and L is CH₂.

In certain embodiments, the anti-inflammatory agent/anti-oxidant agent conjugate is a compound of Formula (A-I) wherein R₁ is hydrogen or acetyl; one of R₂, R₃, R₄, and R₅ is 2,4-difluorophenyl and the rest are hydrogen; R₆ is formula (i); R₇ is hydroxy; R₈ is hydrogen; R₉ is acetyl; X₁ is S; and L is CH₂.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, halo(C₁-C₆)alkyl, or halogen; R₆ is formula (i); R₇ is (C₁-C₆)alkoxy, (C₁-C₆)alkyl, (C₁-C₆)alkylthio, hydroxy, or —NZ₉Z₁₀; R₈ is hydrogen or (C₁-C₆)alkyl; R₉ is (C₁-C₆)alkylcarbonyl; X₁ is O or S; L is (C₁-C₆)alkylene; and Z₉, and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen or halo(C₁-C₆)alkyl; R₆ is formula (i); R₇ is (C₁-C₆)alkoxy or hydroxy; R₈ is hydrogen or (C₁-C₆)alkyl; R₉ is (C₁-C₆)alkylcarbonyl; X₁ is O or S; and L is (C₁-C₆)alkylene.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen or trifluormethyl; R₆ is formula (i); R₇ is ethoxy, methoxy, or hydroxy; R₈ is hydrogen or methyl; R₉ is acetyl; X₁ is O or S; and L is CH₂.

In certain embodiments, the anti-inflammatory agent/anti-oxidant agent conjugate is a compound of Formula (A-I) wherein R₁ is hydrogen or acetyl; one of R₂, R₃, R₄, and R₅ ais trifluormethyl and the rest are hydrogen; R₆ is formula (i); R₇ is hydroxy; R₈ is hydrogen; R₉ is acetyl; X₁ is S; and L is CH₂.

In certain embodiments, the anti-inflammatory agent/anti-oxidant agent conjugate is a compound of Formula (A-I) wherein R₁ is hydrogen or acetyl; R₂, R₃, R₄, and R₅ are hydrogen; and R₆ is (L) N-acetylcysteine, (D) N-acetylcysteine, or (±) N-acetylcysteine

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the anti-inflammatory agent/anti-oxidant agent conjugate is a compound of Formula (A-I) wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyl or Cl.

In certain embodiments, the anti-inflammatory agent/anti-oxidant agent conjugate is a compound of Formula (A-II)

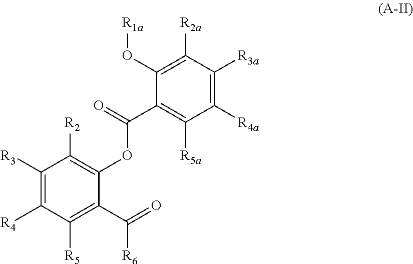

or a pharmaceutically acceptable salt thereof, wherein R₂, R₃, R₄, R₅, R₆, R₁ₐ, R₃ₐ, R₄ₐ, and R₅ₐ are as defined above, provided that when R₆ is hydroxy then R₁, is B.

In certain embodiments, the anti-inflammatory agent/anti-oxidant agent conjugate is a compound of Formula (A-II) wherein R₂, R₃, R₄, R₅, are independently hydrogen, trifluoromethyl, or 2,4-difluorophenyl; R₆ is as defined in Formula (A-I) of the Summary section; R₁ₐ is hydrogen or acetyl; and R₂ₐ, R₃ₐ, R₄ₐ, and R₅ₐ, are independently hydrogen, trifluoromethyl, or 2,4-difluorophenyl.

In certain embodiments, the anti-inflammatory agent/anti-oxidant agent conjugate is a compound of Formula (A-II) wherein R₂, R₃, R₄, R₅, are independently hydrogen, trifluoromethyl, or 2,4-difluorophenyl; R₆ is N-acetylcysteine, (L) N-acetylcysteine, or (D) N-acetylcysteine; R₁, is hydrogen or acetyl; and one of R₂ₐ, R₃ₐ, R₄ₐ, and R₅, is C(O)—R₆ₐ and the rest are hydrogen; and R₆ₐ is as defined in Formula (A-I).

In certain embodiments, the anti-inflammatory agent/anti-oxidant agent conjugate is a compound of Formula (A-III)

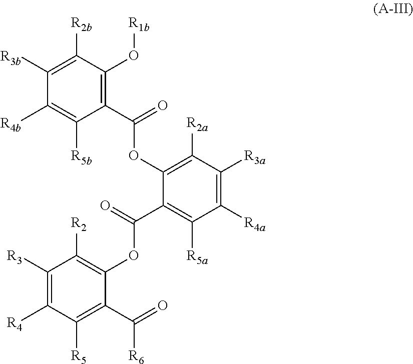

or a pharmaceutically acceptable salt thereof, wherein R₂, R₃, R₄, R₅, R₆, R₂ₐ, R₃ₐ, R₄ₐ, R₅ₐ, R_{1b}, R_{2b}, R_{3b}, R_{4b}, and R_{5b} are as defined in Formula (A-I) above, provided that when R₆ is hydroxy then R_{1b} is C, as defined in Formula (A-I) above.

In certain embodiments, the anti-inflammatory agent/anti-oxidant agent conjugate is a compound of Formula (A-III) wherein R₂, R₃, R₄, R₅, are independently hydrogen, trifluoromethyl, or 2,4-difluorophenyl; R₆ is (L) N-acetylcysteine; R₂ₐ, R₃ₐ, R₄ₐ, and R₅ₐ, are independently hydrogen, trifluoromethyl, or 2,4-difluorophenyl; R_{2b}, R_{3b}, R_{4b}, and R_{5b}, are independently hydrogen, trifluoromethyl, or 2,4-difluorophenyl; and R_{1b} is hydrogen or acetyl.

In certain embodiments, the anti-inflammatory agent/anti-oxidant agent conjugate is a compound of Formula (A-IV)

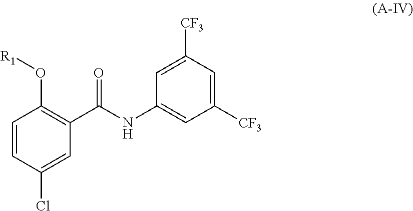

or a pharmaceutically acceptable salt thereof wherein R₁ is hydrogen, (C₁-C₆)alkylcarbonyl,

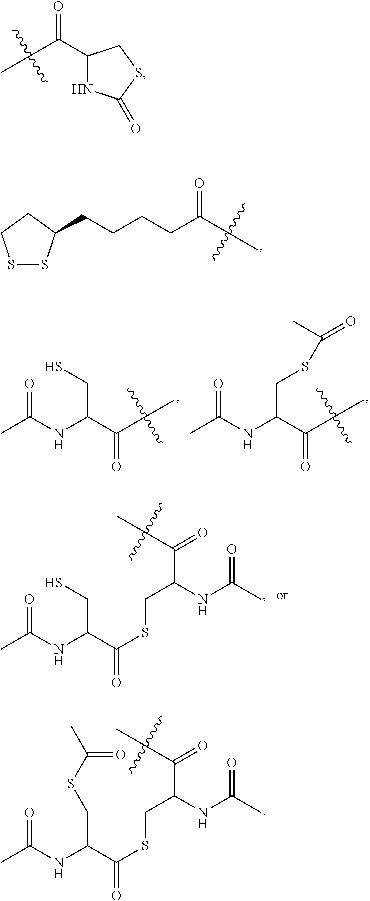

In certain embodiments, the anti-inflammatory agent/anti-oxidant agent conjugate is a compound of Formula (A-V)

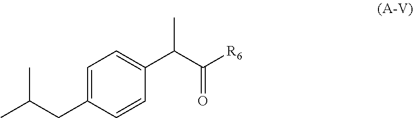

or a pharmaceutically acceptable salt thereof, wherein R₆ is

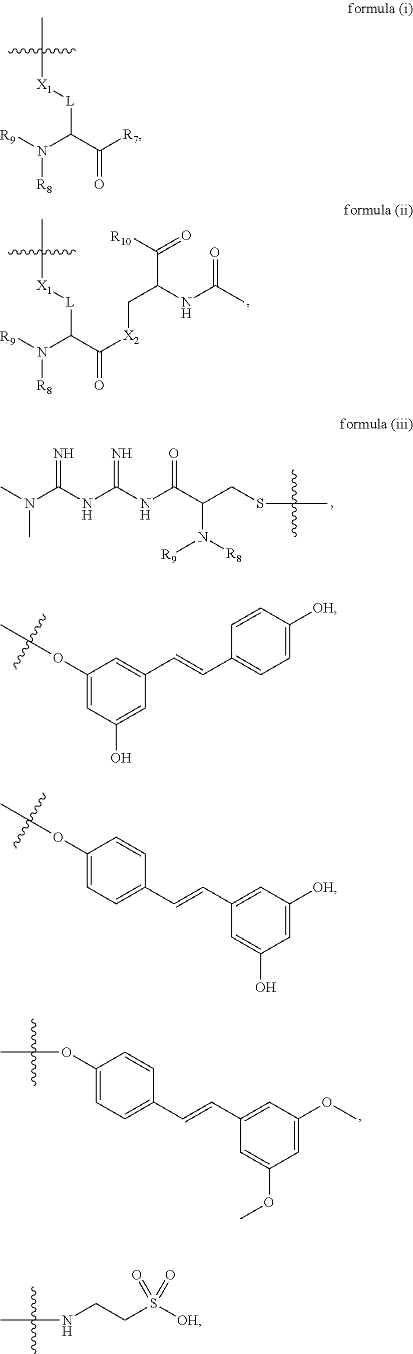

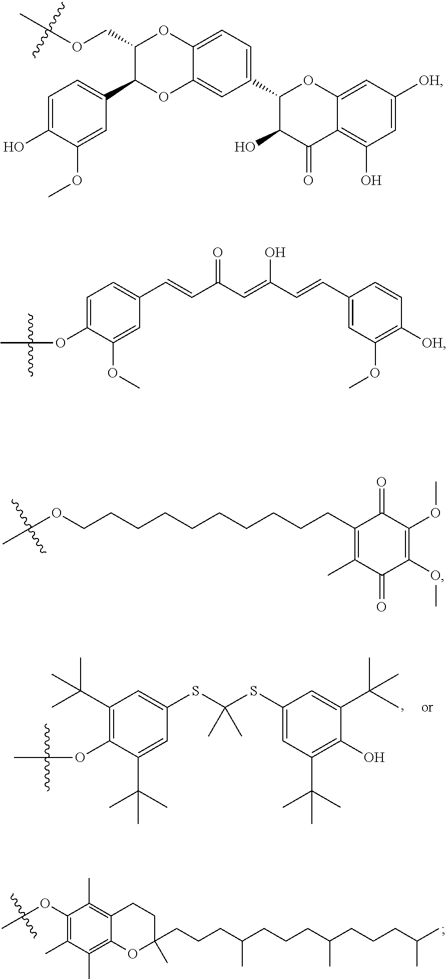 [SMILES: *OC[C@@H]1Oc2ccc([C@@H]3Oc4cc(O)cc(O)c4C(=O)[C@H]3O)cc2O[C@H]1c1ccc(O)c(OC)c1 ; *Oc1ccc(/C=C/C(=O)/C=C(O)/C=C/c2ccc(O)c(OC)c2)cc1OC ; *OCCCCCCCCCCC1=C(C)C(=O)C(OC)=C(OC)C1=O ; *Oc1c(C(C)(C)C)cc(SC(C)(C)Sc2cc(C(C)(C)C)c(O)c(C(C)(C)C)c2)cc1C(C)(C)C ; *Oc1c(C)c(C)c2c(c1C)CCC(C)(CCCC(C)CCCC(C)CCCC(C)C)O2]

R₇ is (C₁-C₆)alkoxy, (C₁-C₆)alkyl, (C₁-C₆)alkylthio, hydroxy, —NZ₉Z₁₀, or O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-VI)

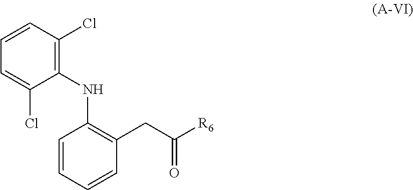

or a pharmaceutically acceptable salt thereof, wherein R₆ is

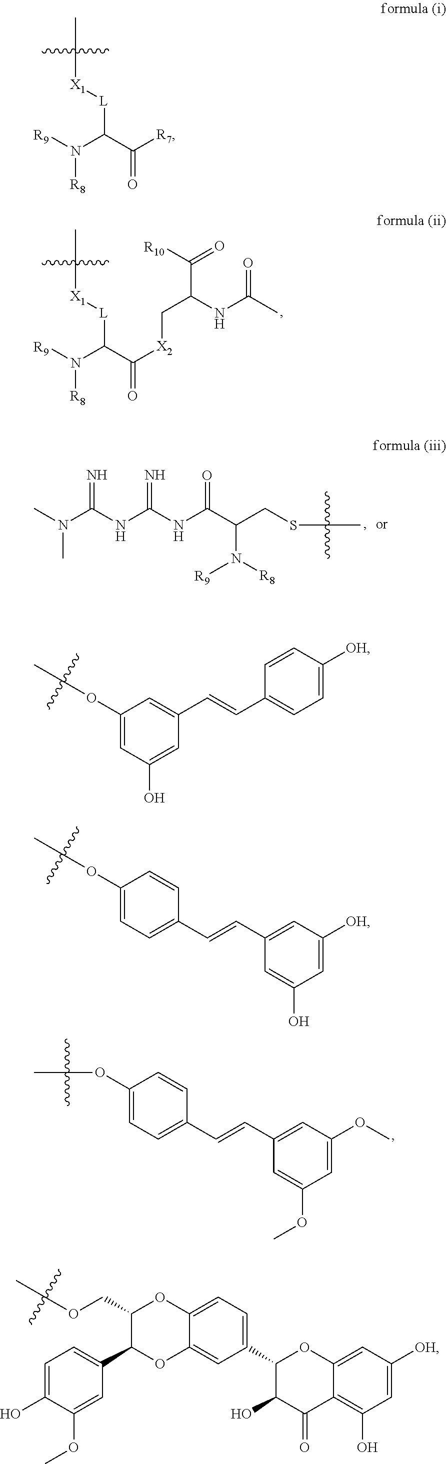

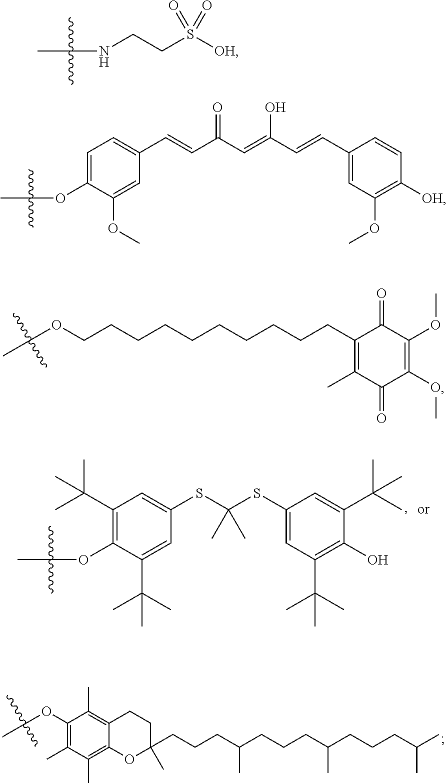

R₇ is (C₁-C₆)alkoxy, (C₁-C₆)alkyl, (C₁-C₆)alkylthio, hydroxy, —NZ₉Z₁₀, or O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-VII)

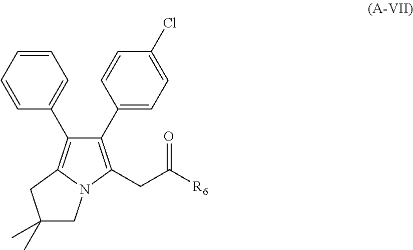

or a pharmaceutically acceptable salt thereof, wherein R₆ is

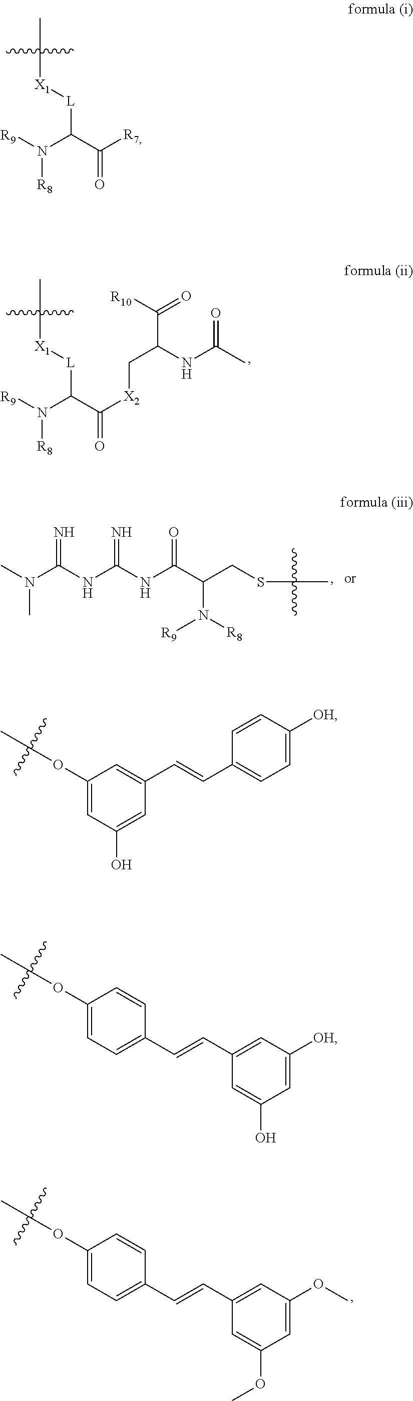

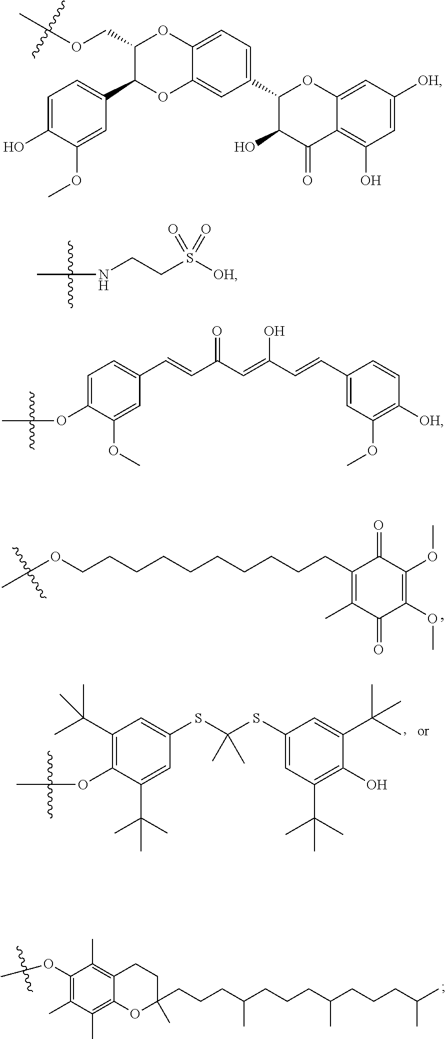

R₇ is (C₁-C₆)alkoxy, (C₁-C₆)alkyl, (C₁-C₆)alkylthio, hydroxy, —NZ₉Z₁₀, or O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-VIII)

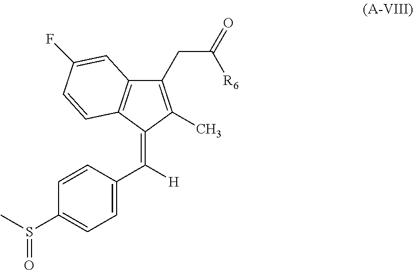

or a pharmaceutically acceptable salt thereof, wherein R₆ is

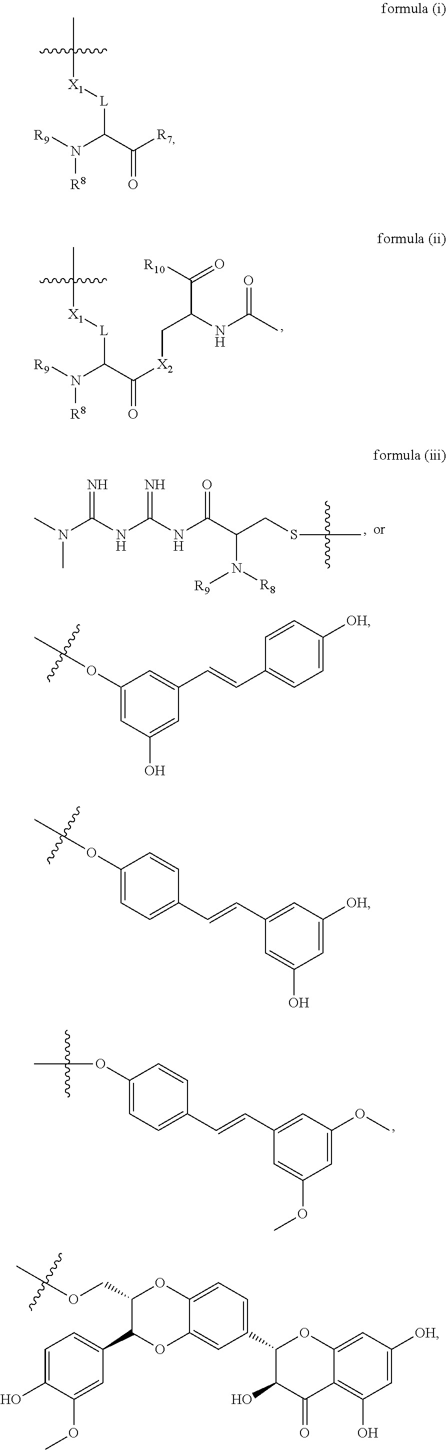

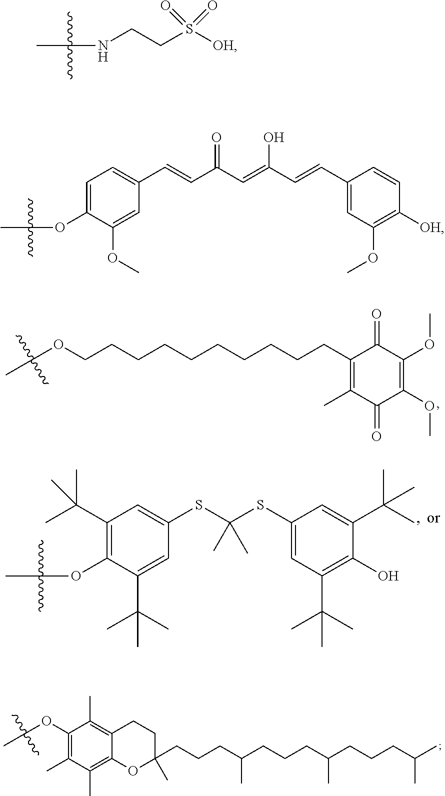

R₇ is (C₁-C₆)alkoxy, (C₁-C₆)alkyl, (C₁-C₆)alkylthio, hydroxy, —NZ₉Z₁₀, or O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-IX)

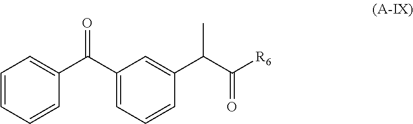

wherein R₆ is

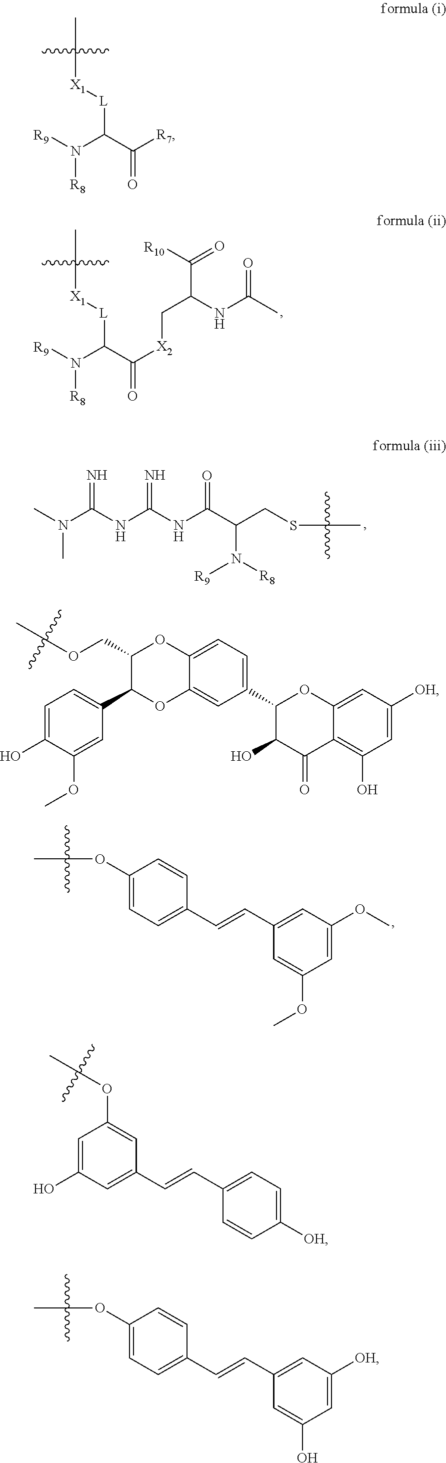

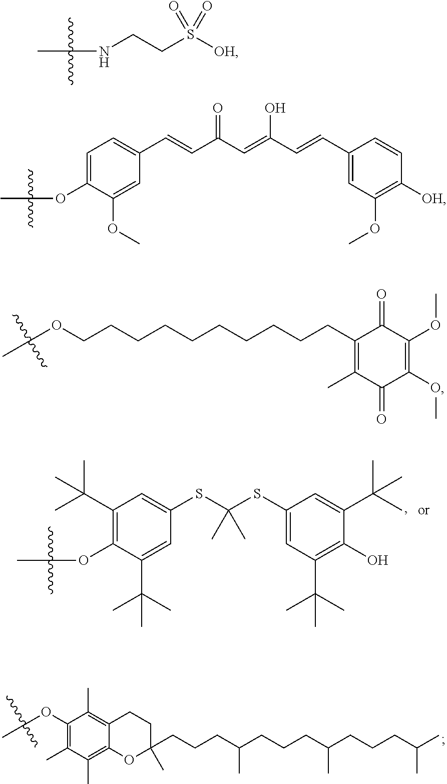

R₇ is (C₁-C₆)alkoxy, (C₁-C₆)alkyl, (C₁-C₆)alkylthio, hydroxy, —NZ₉Z₁₀, or O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀; X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-X)

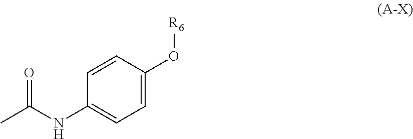

wherein R₆ is

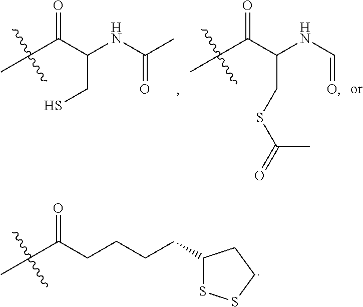

In certain embodiments, the anti-inflammatory agent/anti-oxidant agent conjugate is a compound of Formula (A-XI)

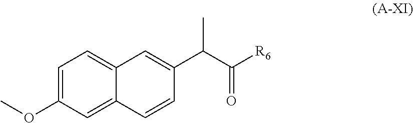

wherein R₆ is

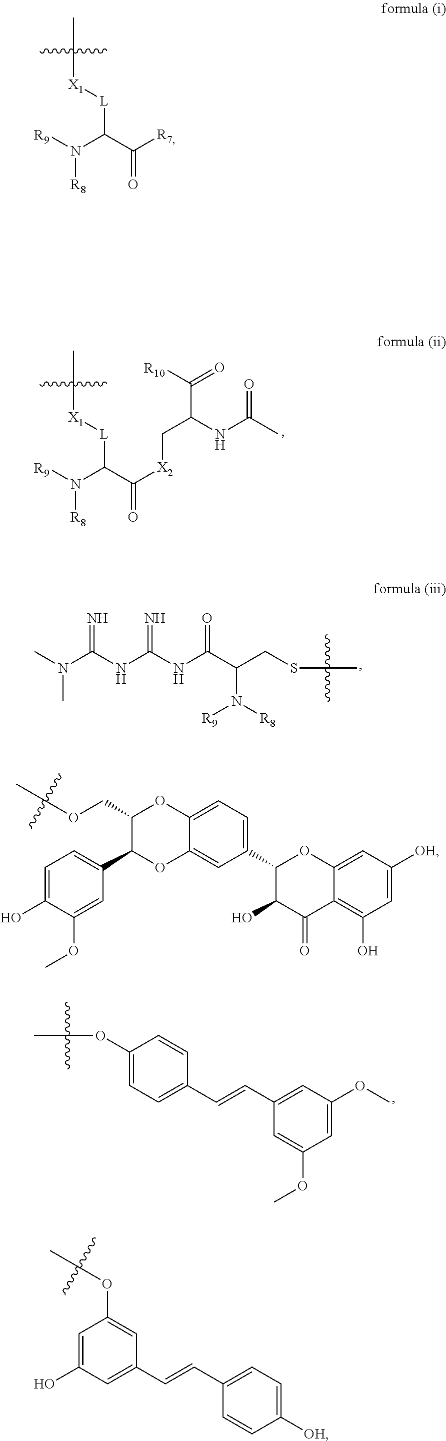

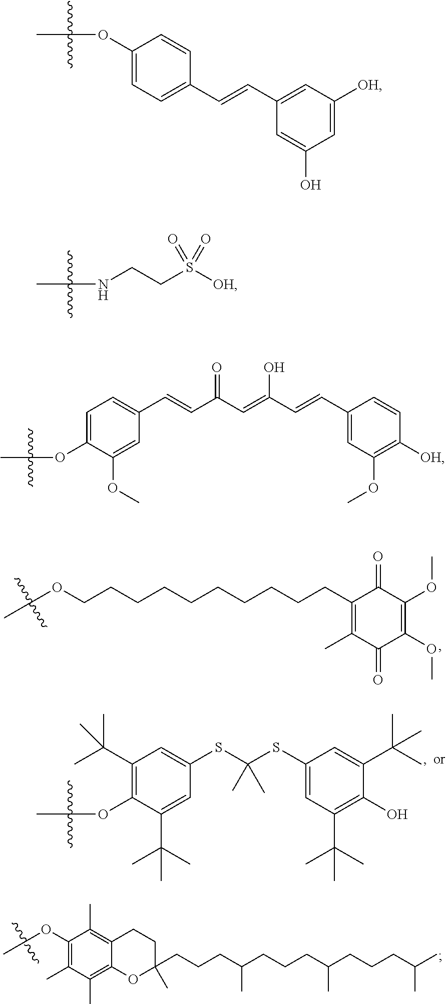

R₇ is (C₁-C₆)alkoxy, (C₁-C₆)alkyl, (C₁-C₆)alkylthio, hydroxy, —NZ₉Z₁₀, or O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

In certain embodiments, the anti-inflammatory agent/anti-oxidant agent conjugate is a compound of Formula (A-XII)

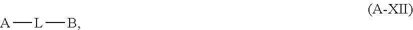

wherein

A is

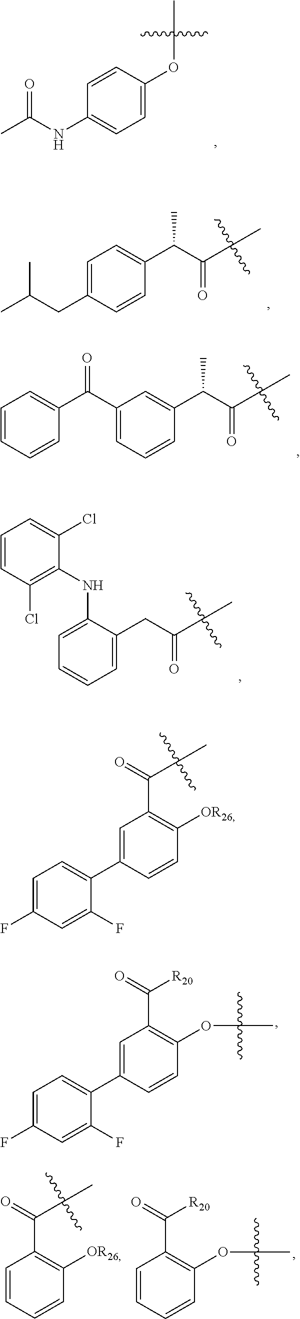

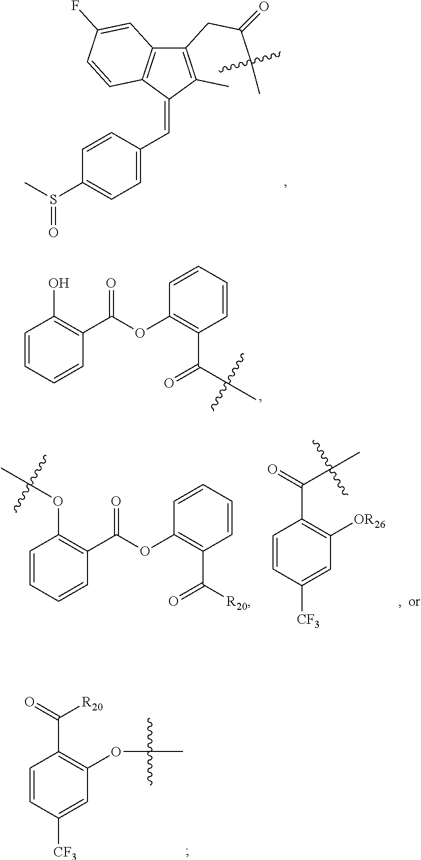

R₂₀ is (C₁-C₄)alkoxy, hydroxy, or NZ₂₀Z₂₁;

Z₂₀ and Z₂₁ are independently hydrogen or (C₁-C₄)alkyl;

L is selected from

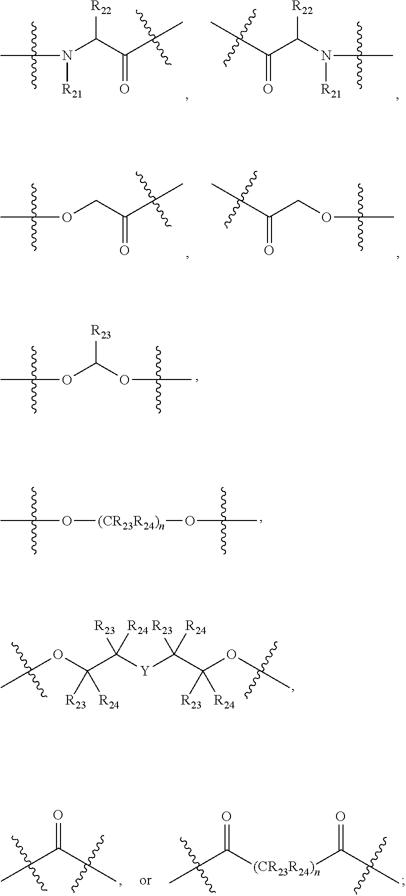

n is 2, 3, or 4;

Y is O, S, S—S, NH, NCH₃;

R₂₁ is hydrogen or (C₁-C₄)alkyl;

R₂₂ is hydrogen, CH₃, CH(CH₃)₂, CH₂CH(CH₃)₂, CH(CH₃)CH₂CH₃; CH₂OH, CH(OH)CH₃, CH₂SH, CH₂COOH, CH₂CH₂COOH, CH₂C(═O)NH₂, CH₂CH₂CH₂NHC(═NH)NH₂, CH₂CH₂CH₂CH₂NH₂, CH₂CH₂SCH₃, CH₂CH₂C(═O)NH₂,

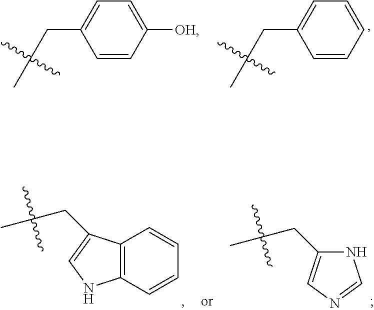 [SMILES: *Cc1ccc(O)cc1 ; *Cc1ccccc1 ; "," ; *Cc1c[nH]c2ccccc12 ; *Cc1cnc[nH]1]

R₂₃ and R₂₄ are independently hydrogen or (C₁-C₆)alkyl;

B is

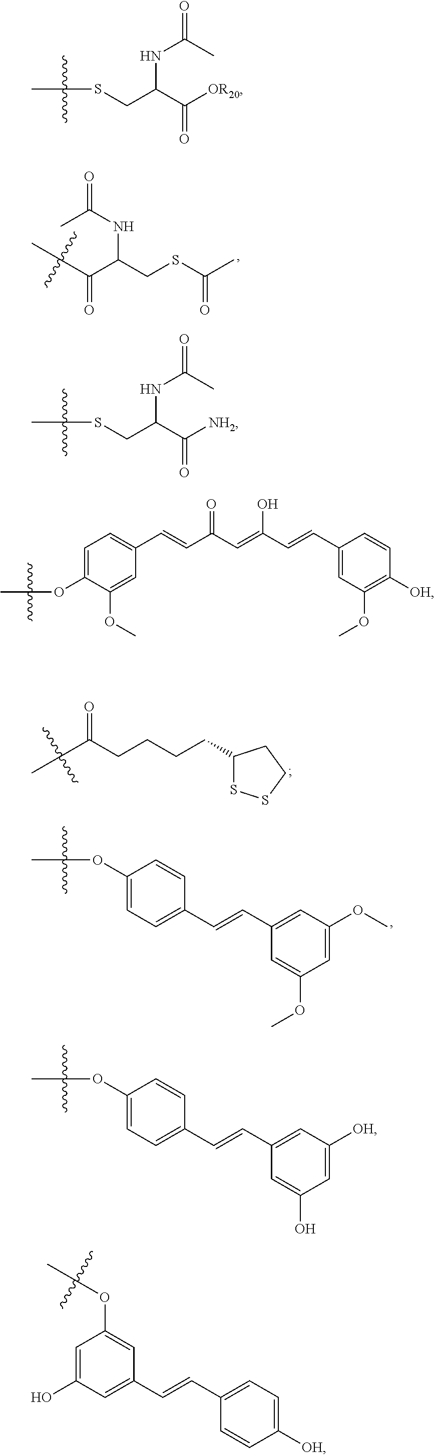

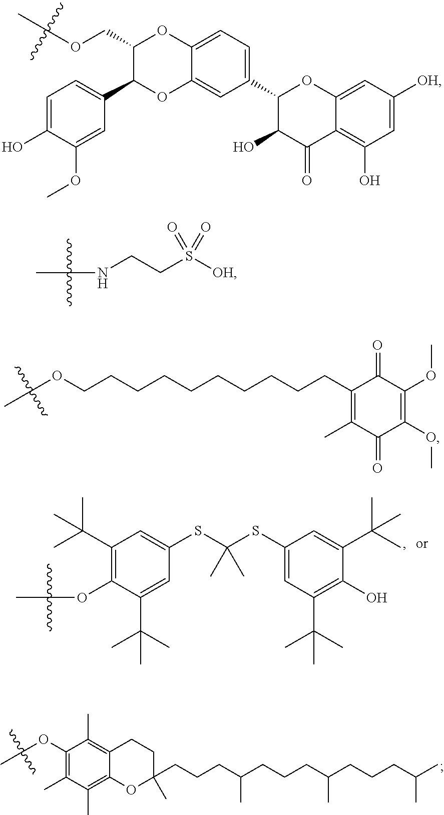

R₂₅ is (C₁-C₄)alkoxy, hydroxy, or —NZ₂₂Z₂₃;

Z₂₂ and Z₂₃ are independently hydrogen or (C₁-C₄)alkyl; and

R₂₆ is hydrogen, (C₁-C₆)alkyl, or (C₁-C₆)alkylcarbonyl.

In certain embodiments, the anti-inflammatory agent/anti-oxidant agent conjugate is a compound of Formula (A-XIII):

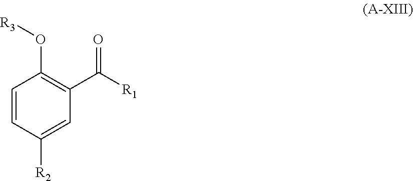

or a pharmaceutically acceptable salt thereof, wherein

R₁ is OR₆ or NR₄R₅;

R₂ is H or 2,4-difluorophenyl;

R₃ is

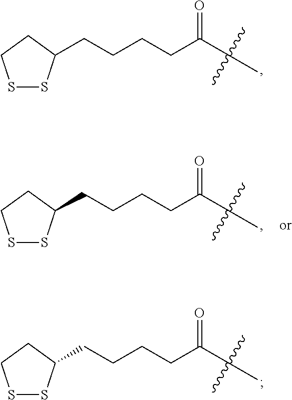 [SMILES: *C(=O)CCCCC1CCSS1 ; *C(=O)CCCC[C@@H]1CCSS1 ; *C(=O)CCCC[C@H]1CCSS1]

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane;

R₆ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; and

Z₁ and Z₂ are independently H or (C₁-C₆)alkyl.

In certain embodiments, the anti-inflammatory agent/anti-oxidant agent conjugate is a compound of Formula (A-XIV)

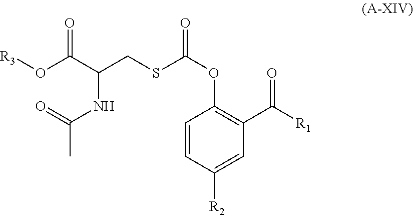

or a pharmaceutically acceptable salt thereof, wherein

R₁ is OR₆ or NR₄R₅;

R₂ is H or 2,4-difluorophenyl;

R₃ is H or (C₁-C₆)alkyl;

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane;

R₆ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; and

Z₁ and Z₂ are independently H or (C₁-C₆)alkyl.

In certain embodiments, the anti-inflammatory agent/anti-oxidant agent conjugate is a compound of Formula (A-XV)

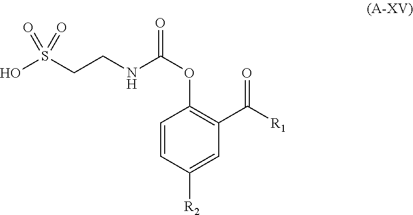

or a pharmaceutically acceptable salt thereof, wherein

R₁ is OR₃ or NR₄R₅;

R₂ is H or 2,4-difluorophenyl;

R₃ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens;

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen) atom to which they are attached form azetidine, pyrrolidine, piperidine, piperazine, N-methylpiperazine, morpholine, or azepane; and

Z₁ and Z₂ are independently H or (C₁-C₆)alkyl.

In certain embodiments, the anti-inflammatory agent/anti-oxidant agent conjugate is a compound of Formula (A-XVI)

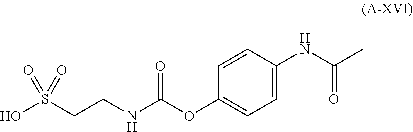 [SMILES: CC(=O)Nc1ccc(OC(=O)NCCS(=O)(=O)O)cc1]

or a pharmaceutically acceptable salt thereof.

In certain embodiments, the anti-inflammatory agent/anti-oxidant agent conjugate is a compound of Formula (A-XVII)

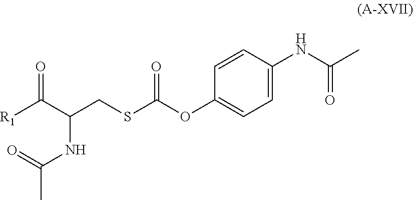

or a pharmaceutically acceptable salt thereof, wherein

R₁ is OR₂ or NR₄R₅;

R₂ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens;

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane; and

Z₁ and Z₂ are independently H or (C₁-C₆)alkyl.

In certain embodiments, the anti-inflammatory agent/anti-oxidant agent conjugate is a compound of Formula (A-XVIII)

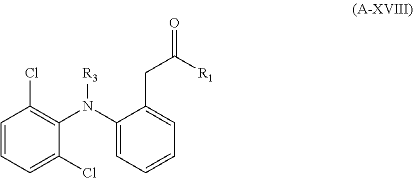

or a pharmaceutically acceptable salt thereof, wherein

R₁ is OR₂ or NR₄R₅;

R₂ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 4, or 5 halogens;

R₃ is

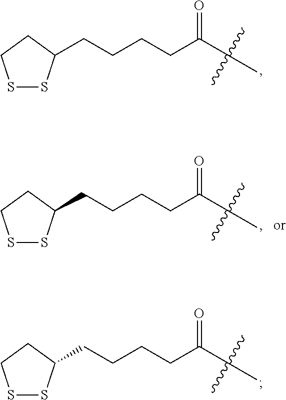 [SMILES: *C(=O)CCCCC1CCSS1 ; *C(=O)CCCC[C@@H]1CCSS1 ; *C(=O)CCCC[C@H]1CCSS1]

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane; and

Z₁ and Z₂ are independently H or (C₁-C₆)alkyl.

In certain embodiments, the anti-inflammatory agent/anti-oxidant agent conjugate is a compound of Formula (A-XIX)

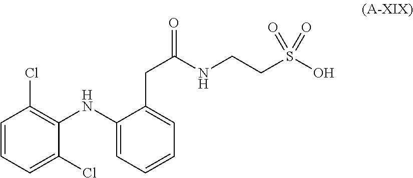 [SMILES: O=C(Cc1ccccc1Nc1c(Cl)cccc1Cl)NCCS(=O)(=O)O]

or a pharmaceutically acceptable salt thereof.

In certain embodiments, the anti-inflammatory agent/anti-oxidant agent conjugate is a compound of Formula (A-XX)

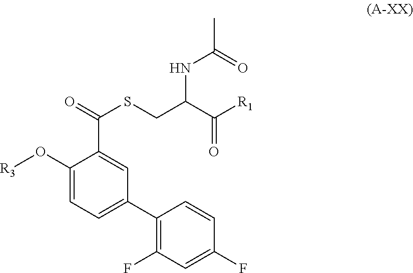

or a pharmaceutically acceptable salt thereof, wherein

R₁ is OR₂, NR₄R₅, or

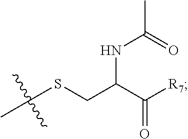

R₂ is (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens;

Z₁ and Z₂ are independently H or (C₁-C₆)alkyl;

R₃ is H or C(O)R₆;

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane;

R₆ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₃Z₄, or phenyl, wherein the phenyl is optionally substituted with 1, 2, 3, 4, or 5 halogens;

Z₃ and Z₄ are independently H or (C₁-C₆)alkyl; and

R₇ is OR, or NR₄R₅.

In certain embodiments, the anti-inflammatory agent/anti-oxidant agent conjugate is a compound of Formula (A-XXI)

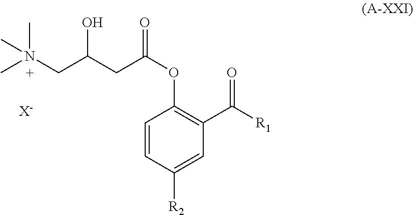

or a pharmaceutically acceptable salt thereof, wherein

X is absent, halogen, HSO₄, HPO₄, CH₃CO₂, or CF₃CO₂;

R₁ is OR₃ or NR₄R₅;

R₂ is H or 2,4-difluorophenyl;

R₃ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens;

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane; and

Z₁ and Z₂ are independently H or (C₁-C₆)alkyl.

In certain embodiments, the anti-inflammatory agent/anti-oxidant agent conjugate is a compound of Formula (A-XXII)

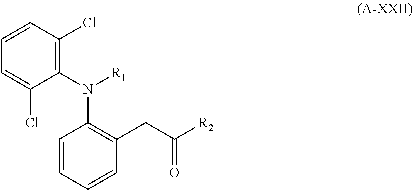

or a pharmaceutically acceptable salt thereof, wherein R₁ is

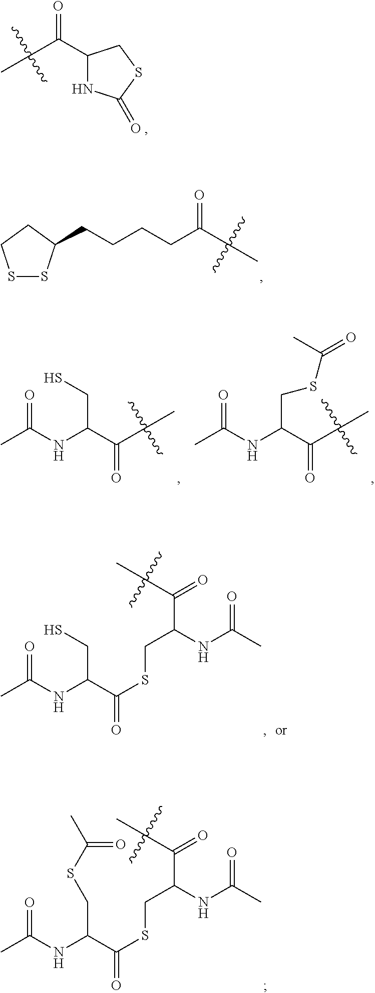

R₂ is (C₁-C₄)alkoxy, hydroxy, or NZ₁Z₂; and

Z₁ and Z₂ are independently hydrogen or (C₁-C₄)alkyl.

In certain embodiments, the anti-inflammatory agent/anti-oxidant agent conjugate is a compound of Formula (B-I)

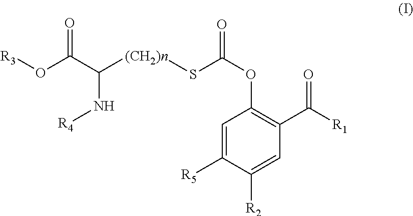

or a pharmaceutically acceptable salt thereof, wherein

n is 1 or 2;

R₁ is OR₆ or NR₆R₇;

R₂ is H or 2,4-difluorophenyl;

R₃ is H or (C₁-C₆)alkyl;

R₄ is H or acetyl;

R₅ is H or trifluoromethyl;

R₆ and R₇ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens, and each Z₁ and Z₂ is independently H or (C₁-C₆)alkyl; or R₆ and R₇ together with the nitrogen atom to which they are attached form azetidine, pyrrolidine, piperidine, piperazine, N-methylpiperazine, morpholine, or azepane.

In certain embodiments of the compounds of Formula (B-I), R₆ is (C₃-C₆)alkyl, (C₃-C₈)cycloalkyl, (C₃-C₈)cycloalkyl(C₁-C₆)alkyl, or aryl(C₁-C₆)alkyl, wherein the alkyl, cycloalkyl, and aryl groups are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₆ and R₇ together with the nitrogen atom to which they are attached form azetidine, pyrrolidine, piperidine, piperazine, N-methylpiperazine, morpholine, or azepane. For example, in one embodiment of the compounds of Formula (B-I), R₆ is (C₃-C₆)alkyl or optionally-substituted benzyl.

In other embodiments of the compounds of Formula (B-I), R₆ is H or (C₁-C₆)alkyl.

In certain embodiments of the compounds of Formula (B-I) as described above, R₁ is OR₆. For example, in one embodiment, R₁ is methoxy, ethoxy or hydroxy. In another embodiment, R₁ is n-propyloxy, i-propyloxy, t-butyoxy, benzyloxy, or 4-methoxybenzyloxy.

In certain embodiments of the compounds of Formula (B-I) as described above, R₁ is NR₆R₇ and R₇ is H, (C₁-C₆)alkyl, (C₃-C₈)cycloalkyl or (C₃-C₈)cycloalkyl(C₁-C₆)alkyl, wherein the alkyl and cycloalkyl groups are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₆ and R₇ together with the nitrogen atom to which they are attached form azetidine, pyrrolidine, piperidine, piperazine, N-methylpiperazine, morpholine, or azepane. For example, in one embodiment, R₇ is H or (C₁-C₆)alkyl.

In certain embodiments of the compounds of Formula (B-I) as described above, R₆ and R₇ are independently (C₁-C₆)alkyl.

In certain embodiments of the compounds of Formula (B-I) as described above, R₁ is amino, methylamino, or dimethylamino.

In certain embodiments of the compounds of Formula (B-I) as described above, R₂ is hydrogen. In other embodiments, R₂ is 2,4-difluorophenyl.

In certain embodiments of the compounds of Formula (B-I) as described above, R₃ is hydrogen or methyl. For example, in one embodiment, R₃ is hydrogen. In another embodiment, R₃ is methyl.

In certain embodiments of the compounds of Formula (B-I) as described above, R₄ is acetyl. In other embodiments, R₄ is H.

In certain embodiments of the compounds of Formula (B-I) as described above, R⁵ is hydrogen.

In certain embodiments of the compounds of Formula (B-I) as described above, R₅ is trifluoromethyl. In certain such embodiments, R₆ is H, methyl or ethyl.

In certain embodiments of the compounds of Formula (B-I) as described above, n is 1.

In certain embodiments of the compounds of Formula (B-I) as described above, n is 2. In certain such embodiments, R₆ is H, methyl or ethyl.

In various embodiments of the compounds of Formula (B-I), substituents and variables are selected from these particular embodiments described above, in several and various combinations thereof.

In certain embodiments, the anti-inflammatory agent/anti-oxidant agent conjugate is a compound of Formula (B-II):

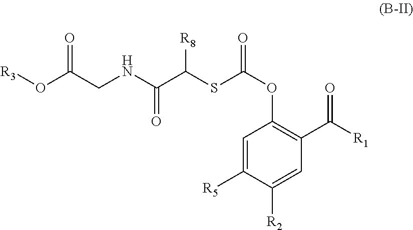

or a pharmaceutically acceptable salt thereof, wherein

R₁ is OR₆ or NR₆R₇;

R₂ is H or 2,4-difluorophenyl;

R₃ is H or (C₁-C₆)alkyl;

R₈ is H or (C₁-C₆)alkyl

R₅ is H or trifluoromethyl;

R₆ and R₇ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens, and each Z₁ and Z₂ is independently H or (C₁-C₆)alkyl; or R₆ and R₇ together with the nitrogen atom to which they are attached form azetidine, pyrrolidine, piperidine, piperazine, N-methylpiperazine, morpholine, or azepane.

In certain embodiments of the compounds of Formula (B-II), R₆ is H or (C₁-C₆)alkyl. In other embodiments, R₆ is (C₃-C₆) alkyl or optionally-substituted benzyl.

In certain embodiments of the compounds of Formula (B-II) as described above, R₁ is OR₆. For example, in one embodiment, R₁ is hydroxy, methoxy, ethoxy n-propyloxy, propyloxy, t-butyoxy, benzyloxy, or 4-methoxybenzyloxy.

In certain embodiments of the compounds of Formula (B-II) as described above, R₁ is NR₆R₇ and R₇ is H, (C₁-C₆)alkyl, (C₃-C₈)cycloalkyl or (C₃-C₈)cycloalkyl(C₁-C₆)alkyl, wherein the alkyl and cycloalkyl groups are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₆ and R₇ together with the nitrogen atom to which they are attached form azetidine, pyrrolidine, piperidine, piperazine, N-methylpiperazine, morpholine, or azepane. For example, in one embodiment, R₇ is H or (C₁-C₆)alkyl.

In certain embodiments of the compounds of Formula (B-II) as described above, R₆ and R₇ are independently H or (C₁-C₆)alkyl.

In certain embodiments of the compounds of Formula (B-II) as described above, R₁ is amino, methylamino, or dimethylamino.

In certain embodiments of the compounds of Formula (B-II) as described above, R₂ is hydrogen. In other embodiments, R₂ is 2,4-difluorophenyl.

In certain embodiments of the compounds of Formula (B-II) as described above, R₃ is hydrogen or methyl. For example, in one embodiment, R₃ is hydrogen. In another embodiment, R₃ is methyl.

In certain embodiments of the compounds of Formula (B-II) as described above, R₈ is acetyl. In other embodiments, R₈ is H.

In certain embodiments of the compounds of Formula (B-II) as described above, R₅ is hydrogen.

In certain embodiments of the compounds of Formula (B-II) as described above, R₅ is trifluoromethyl. In certain such embodiments, R₆ is H, methyl or ethyl.

In various embodiments of the compounds of Formula (B-II), substituents and variables are selected from these particular embodiments described above, in several and various combinations thereof.

In certain embodiments, the anti-inflammatory agent/anti-oxidant agent conjugate is a compound of Formula (B-III)

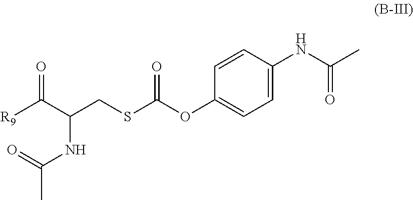

or a pharmaceutically acceptable salt thereof, wherein

R₉ is OR₃ or NR₁₀R₁ₗ;

R₃ is H, (C₁-C₆)alkyl, (C₃-C₈)cycloalkyl, or (C₃-C₈)cycloalkyl(C₁-C₆)alkyl, wherein the (C₁-C₆)alkyl, and (C₃-C₈)cycloalkyl, (C₃-C₈)cycloalkyl(C₁-C₆)alkyl are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens;

R₁₀ and R₁₁ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane;

in which each Z₁ and Z₂ is independently H or (C₁-C₆)alkyl.

In certain embodiments of the compounds of Formula (B-III), R₉ is OR₃, and R₃ is (C₃-C₆)alkyl, (C₃-C₈)cycloalkyl, or (C₃-C₈)cycloalkyl(C₁-C₆)alkyl, wherein the alkyl and cycloalkyl groups are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens. For example, in certain embodiments, R₉ is n-propyloxy, i-propyloxy, t-butyoxy, benzyloxy, or 4-methoxybenzyloxy.

In certain embodiments of the compounds of Formula (B-III), R₉ is NR₁₀R₁₁; and R₁₀ is (C₂-C₆)alkyl, (C₃-C₈)cycloalkyl, or (C₃-C₈)cycloalkyl(C₁-C₆)alkyl, wherein the alkyl and cycloalkyl groups are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; and R₁₁ is H, (C₁-C₆)alkyl, (C₃-C₈)cycloalkyl, or (C₃-C₈)cycloalkyl(C₁-C₆)alkyl, wherein the alkyl and cycloalkyl groups are independently optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens, or R₁₀ and R₁₁ together with the nitrogen atom to which they are attached form azetidine, pyrrolidine, piperidine, piperazine, N-methylpiperazine, morpholine, or azepane. For example, in certain such embodiments, R₉ is NR₁₀R₁₁, R₁₀ is (C₂-C₆)alkyl and R₁, is H or (C₁-C6)alkyl.

In certain embodiments, the anti-inflammatory agent/anti-oxidant agent conjugate is a compound of Formula (C-I)

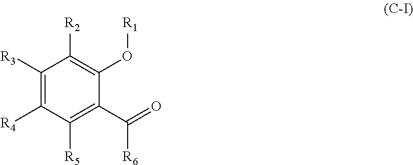

or a pharmaceutically acceptable salt thereof, wherein

R₁ is hydrogen, (C₁-C₆)alkylcarbonyl, or A;

R₂, R₃, R₄, and R₅ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁Z₂, or (NZ₁Z₂)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, (NZ₃Z₄)carbonyl;

Z₁, Z₂, Z₃, and Z₄ are independently hydrogen, (C₁-C₆)alkyl, or (C₁-C₆)alkylcarbonyl;

R₆ is

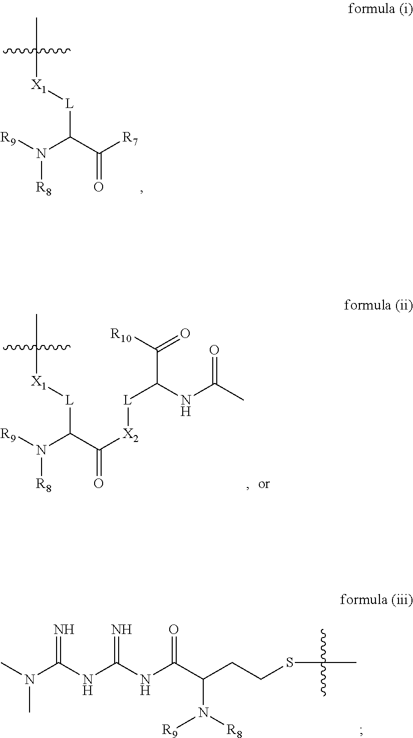

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkyl, (C₁-C₆)alkylthio, hydroxy, or —NZ₉Z₁₀;

Z₉ and Z₁₀ are independently hydrogen, (C₁-C₆)alkyl, or (C₁-C₆)alkylcarbonyl;

X₁ and X₂ are independently O or S;

L is CH₂CH₂;

A is

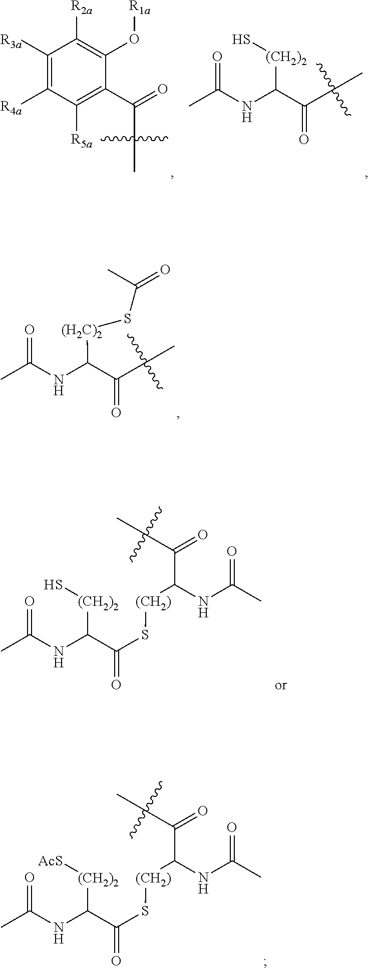

R₁ₐ is hydrogen, (C₁-C₆)alkylcarbonyl, or B;

R₂, R₃ₐ, R₄ₐ, and R₅ₐ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁ₐZ₂ₐ, or (NZ₁ₐZ₂ₐ)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃ₐZ₄ₐ, or (NZ₃ₐZ₄ₐ)carbonyl;

Z₁ₐ, Z₂ₐ, Z₃ₐ, and Z₄ₐ are independently hydrogen, (C₁-C₆)alkyl, or (C₁-C₆)alkylcarbonyl;

B is

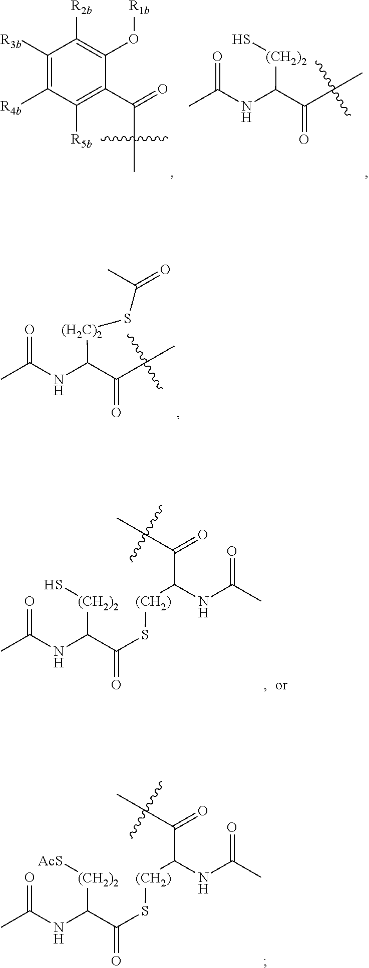

R_{1b} is hydrogen, (C₁-C₆)alkylcarbonyl, or C;

R_{2b}, R_{3b}, R_{4b}, and R_{5b} are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{1b}Z_{2b}, or (NZ_{1b}Z_{2b})carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{3b}Z_{4b}, or (NZ_{3b}Z_{4b})carbonyl;

Z_{1b}, Z_{2b}, Z_{3b}, and Z_{4b} are independently hydrogen, (C₁-C₆)alkyl, or (C₁-C₆)alkylcarbonyl; and

C is

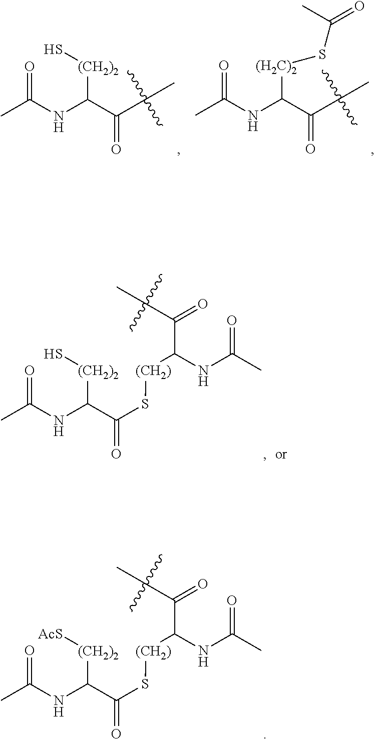

In certain such compounds, X₁ and X₂ are S.

In certain embodiments of the compounds described herein with respect to Formula (C-I), R₂, R₃, R₄ and R₅ are H. In certain such embodiments, R₂ₐ, R_{2b}, R₃ₐ, R_{3b}, R₄ₐ, R_{4b}, R₅ₐ and R_{5b} are H.

In certain embodiments of the compounds described herein with respect to Formula (C-I), R₃ is trifluoromethyl, and R₂, R₄ and R₅ are H. In certain such embodiments, R₃ₐ and R_{3b} are trifluoromethyl, and R₂ₐ, R₄ₐ, R₅ₐ, R_{2b}, R_{4b} and R_{5b} are H.

In certain embodiments of the compounds described herein with respect to Formula (C-I), R₄ is 2,4-difluorophenyl, and R₂, R₃ and R₅ are H. In certain such embodiments, R₄ₐ and R_{4b} are 2,4-difluorophenyl, and R₂ₐ, R₃ₐ, R₅ₐ, R_{2b}, R_{3b} and R_{5b} are H.

In certain embodiments, the anti-inflammatory agent/anti-oxidant agent conjugate is a compound of Formula (C-I) wherein R₁ is hydrogen or acetyl; R₂, R₃, R₄, and R₅ are independently hydrogen, halo(C₁-C₆)alkyl, halogen, or phenyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, or (N₃Z₄)carbonyl; R₆ is formula (i); R₇ is (C₁-C₆)alkoxy, (C₁-C₆)alkyl, (C₁-C₆)alkylthio, hydroxy, or —NZ₉Z₁₀; R₈ is hydrogen or (C₁-C₆)alkyl; R₉ is (C₁-C₆)alkylcarbonyl; X₁ is O or S; and Z₃, Z₄, Z₉, and ₁₀ are independently hydrogen, (C₁-C₆)alkyl, or (C₁-C₆)alkylcarbonyl. In certain such embodiments, X₁ is S.

In certain embodiments, the anti-inflammatory agent/anti-oxidant agent conjugate is a compound of Formula (C-I) wherein R₁ is hydrogen or acetyl; R₂, R₃, R₄, and R₅ are independently hydrogen or phenyl, wherein the phenyl is optionally substituted with 1 or 2 groups that are independently halo(C₁-C₆)alkoxy, halo(C₁-C₆)alkyl, or halogen; R₆ is formula (i); R₇ is (C₁-C₆)alkoxy or hydroxy; R₈ is hydrogen or (C₁-C₆)alkyl; R₉ is (C₁-C₆)alkylcarbonyl; and X₁ is O or S. In certain such embodiments, X₁ is S.

In certain embodiments, the anti-inflammatory agent/anti-oxidant agent conjugate is a compound of Formula (C-I) wherein R₁ is hydrogen or acetyl; R₂, R₃, R₄, and R₅ are independently hydrogen or phenyl, wherein the phenyl is optionally substituted with 1 or 2 halogen groups; R₆ is formula (i); R₇ is ethoxy, methoxy, or hydroxy; R₈ is hydrogen or methyl; R₉ is acetyl; and X₁ is O or S. In certain such embodiments, X₁ is S.

In certain embodiments, the anti-inflammatory agent/anti-oxidant agent conjugate is a compound of Formula (C-I) wherein R₁ is hydrogen or acetyl; one of R₂, R₃, R₄, and R₅ is 2,4-difluorophenyl and the rest are hydrogen; R₆ is formula (i); R₇ is hydroxy; R₈ is hydrogen; R₉ is acetyl; and X₁ is S₂.

In certain embodiments, the anti-inflammatory agent/anti-oxidant agent conjugate is a compound of Formula (C-I) wherein R₁ is hydrogen or acetyl; R₂, R₃, R₄, and R₅ are independently hydrogen, halo(C₁-C₆)alkyl, or halogen; R₆ is formula (i); R₇ is (C₁-C₆)alkoxy, (C₁-C₆)alkyl, (C₁-C₆)alkylthio, hydroxy, or —NZ₉Z₁₀; R₈ is hydrogen or (C₁-C₆)alkyl; R₉ is (C₁-C₆)alkylcarbonyl; X₁ is O or S; and Z₉, and Z₁₀ are independently hydrogen, (C₁-C₆)alkyl, or (C₁-C₆)alkylcarbonyl. In certain such embodiments, X₁ is S.

In certain embodiments, the anti-inflammatory agent/anti-oxidant agent conjugate is a compound of Formula (C-I) wherein R₁ is hydrogen or acetyl; R₂, R₃, R₄, and R₅ are independently hydrogen or halo(C₁-C₆)alkyl; R₆ is formula (i); R₇ is (C₁-C₆)alkoxy or hydroxy; R₈ is hydrogen or (C₁-C₆)alkyl; R₉ is (C₁-C₆)alkylcarbonyl; and X₁ is O or S. In certain such embodiments, X₁ is S.

In certain embodiments, the anti-inflammatory agent/anti-oxidant agent conjugate is a compound of Formula (C-I) wherein R₁ is hydrogen or acetyl; R₂, R₃, R₄, and R₅ are independently hydrogen or trifluormethyl; R₆ is formula (i); R₇ is ethoxy, methoxy, or hydroxy; R₈ is hydrogen or methyl; R₉ is acetyl; and X₁ is O or S. In certain such embodiments, X₁ is S.

In certain embodiments, the anti-inflammatory agent/anti-oxidant agent conjugate is a compound of Formula (C-I) wherein R₁ is hydrogen or acetyl; one of R₂, R₃, R₄, and R₅ ais trifluormethyl and the rest are hydrogen; R₆ is formula (i); R₇ is hydroxy; R₈ is hydrogen; R₉ is acetyl; and X₁ is S.

In certain embodiments, the anti-inflammatory agent/anti-oxidant agent conjugate is a compound of Formula (C-II)

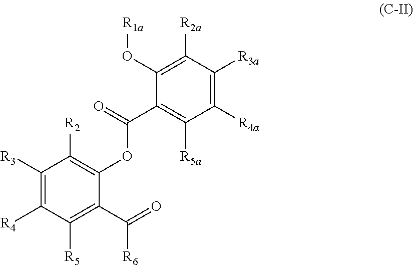

wherein R₂, R₃, R₄, R₅, R₆, R₁ₐ, R₂ₐ, R₃ₐ, R₄ₐ, and R₅ₐ are as defined in Formula (C-I) above, provided that when R₆ is hydroxy then R₁ₐ is B, as defined in Formula (C-I) above.

In certain embodiments, the anti-inflammatory agent/anti-oxidant agent conjugate is a compound of Formula (C-II) wherein R₂, R₃, R₄, R₅, are independently hydrogen, trifluoromethyl, or 2,4-difluorophenyl; R₆ is as defined in Formula (C-I) above; R₁ₐ is hydrogen or acetyl; and R₂ₐ, R₃ₐ, R₄ₐ, and R₅ₐ, are independently hydrogen, trifluoromethyl, or 2,4-difluorophenyl.

In certain embodiments, the anti-inflammatory agent/anti-oxidant agent conjugate is a compound of Formula (C-II) wherein R₂, R₃, R₄, R₅, are independently hydrogen, trifluoromethyl, or 2,4-difluorophenyl; R₆ is (S)-3-acetamido-3-carboxypropylthio; (R)-3-acetamido-3-carboxypropylthio or (+/−)-3-acetamido-3-carboxypropylthio; R₁ₐ is hydrogen or acetyl; and one of R₂ₐ, R₃ₐ, R₄ₐ, and R₅ₐ is C(O)—R₆ₐ and the rest are hydrogen; and R₆ₐ is as defined in Formula (C-I).

In certain such compounds, X₁ and X₂ are S.

In certain embodiments of the compounds described herein with respect to Formula (C-II), R₂, R₃, R₄ and R₅, R₂ₐ, R₃ₐ, R₄ₐ and R₅ₐ are H. In certain such embodiments, R_{2b}, R_{3b}, R_{4b} and R_{5b} are H.

In certain embodiments of the compounds described herein with respect to Formula (C-II), R₃ and R₃ₐ are trifluoromethyl, and R₂, R₂ₐ, R₄, R₄ₐ, R₅ and R₅ₐ are H. In certain such embodiments, R_{3b} is trifluoromethyl, and R_{2b}, R_{4b} and R_{5b} are H.

In certain embodiments of the compounds described herein with respect to Formula (C-II), R₄ and R₄ₐ are 2,4-difluorophenyl, and R₂, R₃. R₅ R₂ₐ, R₃ₐ and R₅ₐ are H. In certain such embodiments, R_{4b} is 2,4-difluorophenyl, and R_{2b}, R_{3b} and R_{5b} are H.

In another aspect, the present invention provides conjugates of Formula (C-III)

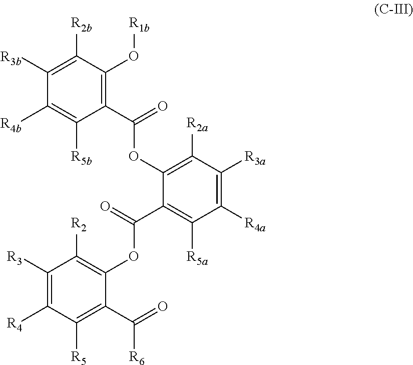

wherein R₂, R₃, R₄, R₅, R₆, R₂ₐ, R₃ₐ, R₄ₐ, R₅ₐ, R_{1b}, R_{2b}, R_{3b}, R_{4b}, and R_{5b} are as defined in Formula (C-I) above, provided that when R₆ is hydroxy then R_{1b} is C, as defined in Formula (C-I) above.

In certain embodiments, the anti-inflammatory agent/anti-oxidant agent conjugate is a compound of Formula (C-III) wherein R₂, R₃, R₄, R₅, are independently hydrogen, trifluoromethyl, or 2,4-difluorophenyl; R₆ is (S)-3-acetamido-3-carboxypropylthio; R₂ₐ, R₃ₐ, R₄ₐ, and R₅ₐ, are independently hydrogen, trifluoromethyl, or 2,4-difluorophenyl; R_{2b}, R_{3b}, R_{4b}, and R_{5b}, are independently hydrogen, trifluoromethyl, or 2,4-difluorophenyl; and R_{1b} is hydrogen or acetyl.

In certain such compounds, X₁ and X₂ are S.

In certain embodiments of the compounds described herein with respect to Formula (C-III), R₂, R₃, R₄ and R₅, R₂ₐ, R₃ₐ, R₄ₐ, R₅ₐ R_{2b}, R_{3b}, R_{4b} and R_{5b} are H.

In certain embodiments of the compounds described herein with respect to Formula (C-III), R₃, R₃ₐ and R_{3b} are trifluoromethyl, and R₂, R₂ₐ, R_{2b}, R₄, R₄ₐ, R_{4b}, R₅, R₅ₐ and R_{5b} are H.

In certain embodiments of the compounds described herein with respect to Formula (C-III), R₄, R₄ₐ and R_{4b} are 2,4-trifluoromethyl, and R₂, R₂ₐ, R_{2b}, R₃, R₃ₐ, R_{3b}, R₅, R₅ₐ and R_{5b} are H.

In certain embodiments, the anti-inflammatory agent/anti-oxidant agent conjugate is a compound of Formula (C-IV)

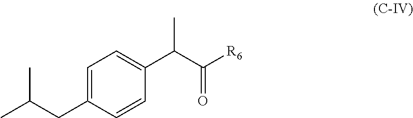

wherein R₆ is

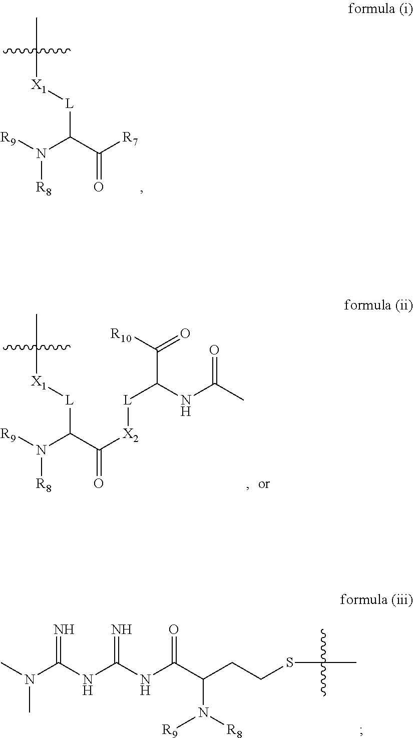

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is CH₂CH₂; and

Z₉ and Z₁₀ are independently hydrogen, (C₁-C₆)alkyl, or (C₁-C₆)alkylcarbonyl.

In certain such compounds, X₁ and X₂ are S.

In certain embodiments, the anti-inflammatory agent/anti-oxidant agent conjugate is a compound of Formula (C-V)

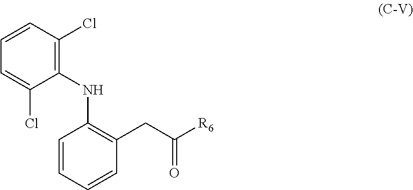

wherein R₆ is

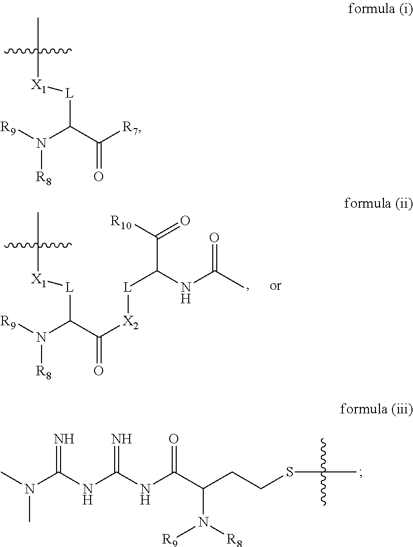

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is CH₂CH₂; and

Z₉ and Z₁₀ are independently hydrogen, (C₁-C₆)alkyl, or (C₁-C₆)alkylcarbonyl.

In certain such embodiments, X₁ and X₂ are S.

In certain embodiments, the anti-inflammatory agent/anti-oxidant agent conjugate is a compound of Formula (C-VI)

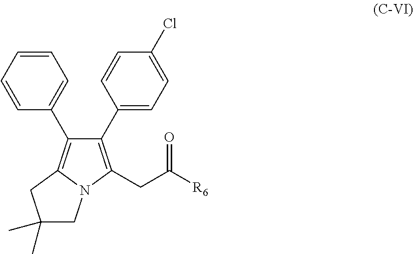

wherein R₆ is

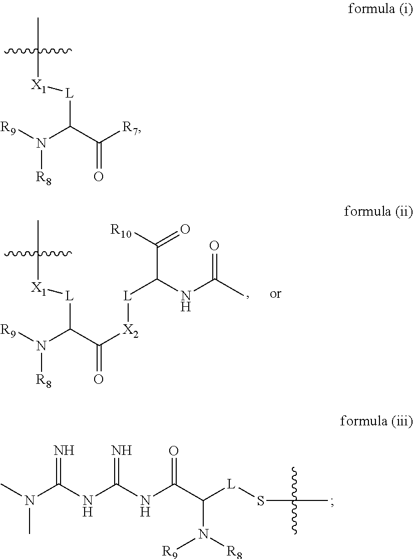

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is CH₂CH₂; and

Z₉ and Z₁₀ are independently hydrogen, (C₁-C₆)alkyl, or (C₁-C₆)alkylcarbonyl.

In certain such embodiments, X₁ and X₂ are S.

In certain embodiments, the anti-inflammatory agent/anti-oxidant agent conjugate is a compound of Formula (C-VII)

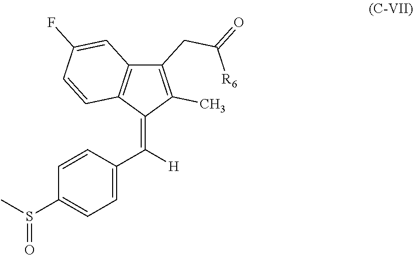

wherein R₆ is

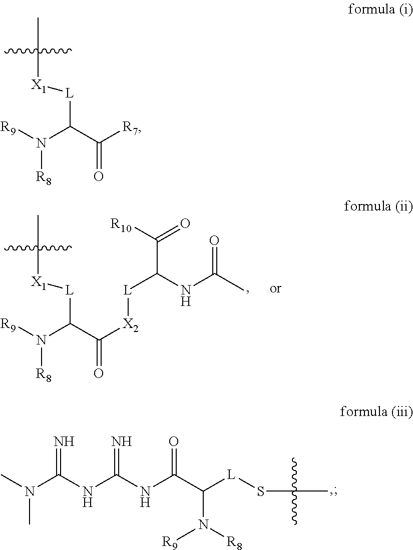

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is CH₂CH₂; and

Z₉ and Z₁₀ are independently hydrogen, (C₁-C₆)alkyl, or (C₁-C₆)alkylcarbonyl.

In certain such embodiments, X₁ and X₂ are S.

In certain embodiments, the anti-inflammatory agent/anti-oxidant agent conjugate is a compound of Formula (C-VIII)

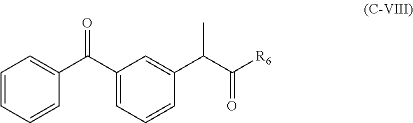

wherein R₆ is

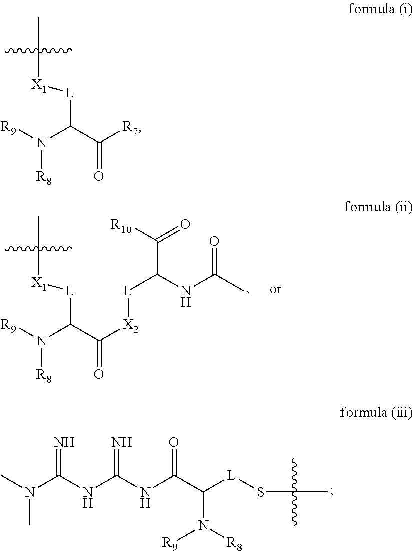

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is CH₂CH₂; and

Z₉ and Z₁₀ are independently hydrogen, (C₁-C₆)alkyl, or (C₁-C₆)alkylcarbonyl.

In certain such embodiments, X₁ and X₂ are S.

In certain embodiments, the anti-inflammatory agent/anti-oxidant agent conjugate is a compound of Formula (C-IX)

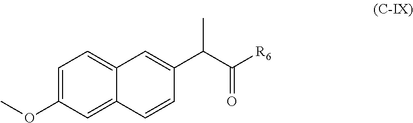

wherein R₆ is

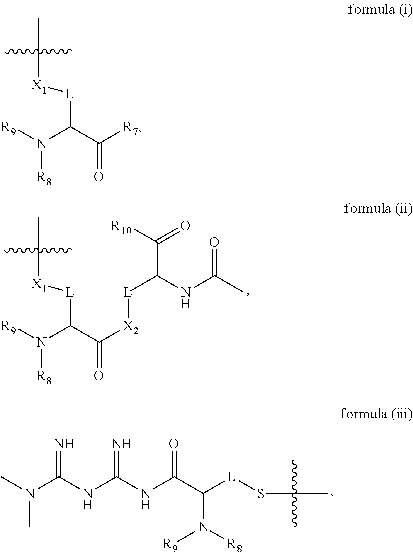

R₇ is (C₁-C₆)alkoxy, (C₁-C₆)alkyl, (C₁-C₆)alkylthio, hydroxy, —NZ₉Z₁₀, or —O-phenyl, wherein the phenyl is optionally substituted with 1, 2, 3, 4, or 5 groups that are independently (C₁-C₆)alkoxy, (C₁-C₆)alkoxycarbonyl, (C₁-C₆)alkyl, (C₁-C₆)alkylcarbonyl, (C₁-C₆)alkylcarbonyloxy, carboxy, cyano, formyl, halo(C₁-C₆)alkoxy, halo(C₁-C₆)alkyl, halogen, hydroxy, or hydroxy(C₁-C₆)alkyl;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is CH₂CH₂; and

Z₉ and Z₁₀ are independently hydrogen, (C₁-C₆)alkyl, or (C₁-C₆)alkylcarbonyl.

In certain such embodiments, X₁ and X₂ are S.

In certain embodiments, the anti-inflammatory agent/anti-oxidant agent conjugate is a compound of Formula (C-X)

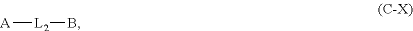

wherein

A is

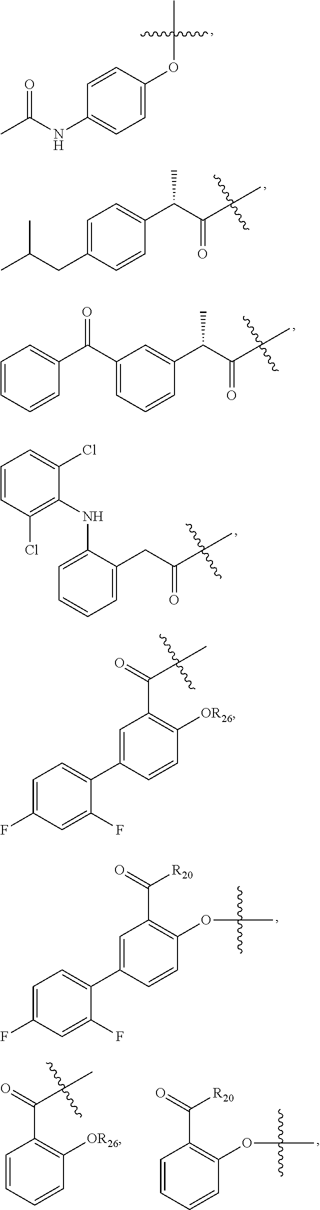

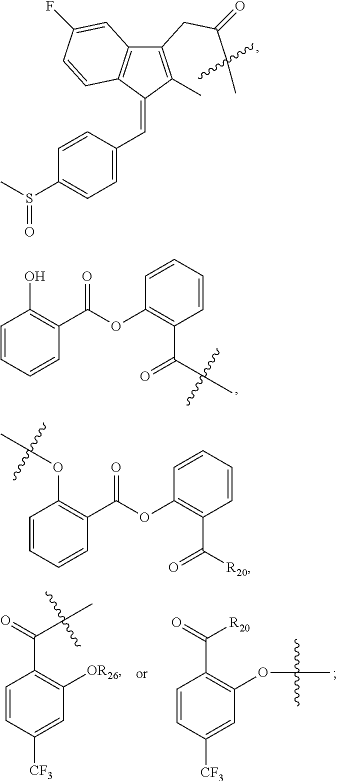

R₂₀ is (C₁-C₄)alkoxy, hydroxy, or —NZ₂₀Z₂₁;

Z₂₀ and Z₂₁ are independently hydrogen or (C₁-C₄)alkyl;

L₂ is selected from

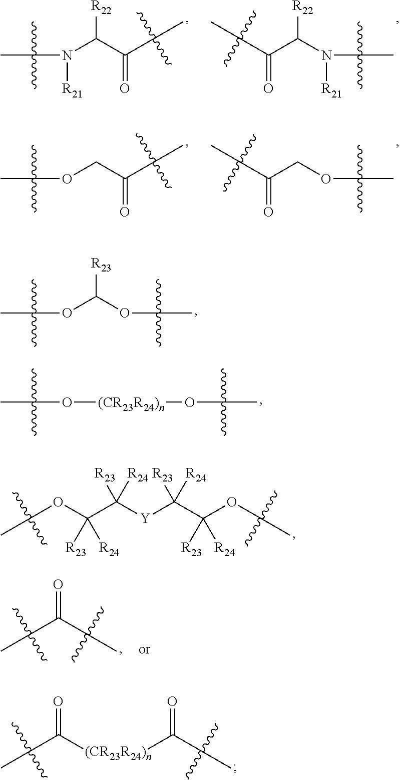

n is 2, 3, or 4;

Y is O, S, S—S, NH, NCH₃;

R₂₁ is hydrogen or (C₁-C₄)alkyl;

R₂₂ is hydrogen, CH₃, CH(CH₃)₂, CH₂CH(CH₃)₂, CH(CH₃)CH₂CH₃; CH₂OH, CH(OH)CH₃, CH₂SH, CH₂COOH, CH₂CH₂COOH, CH₂C(═O)NH₂, CH₂CH₂CH₂NHC(═NH)NH₂, CH₂CH₂CH₂CH₂NH₂, CH₂CH₂SCH₃, CH₂CH₂C(═O)NH₂,

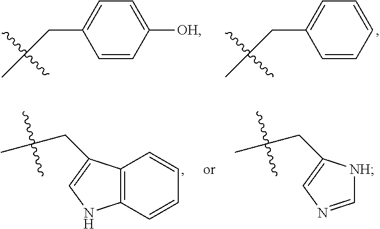 [SMILES: *Cc1ccc(O)cc1 ; *Cc1ccccc1 ; *Cc1c[nH]c2ccccc12 ; *Cc1cnc[nH]1]

R₂₃ and R₂₄ are independently hydrogen or (C₁-C₆)alkyl;

B is

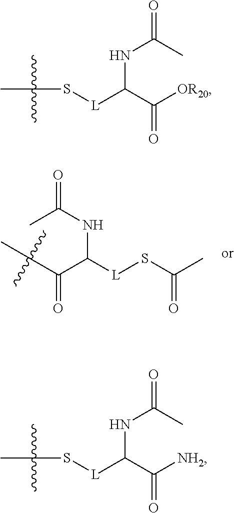

in which L is CH₂CH₂

R₂₅ is (C₁-C₄)alkoxy, hydroxy, or —NZ₂₂Z₂₃;

Z₂₂ and Z₂₃ are independently hydrogen or (C₁-C₄)alkyl; and

R₂₆ is hydrogen, (C₁-C₆)alkyl, or (C₁-C₆)alkylcarbonyl.

In certain embodiments, the anti-inflammatory agent/anti-oxidant agent conjugate is a compound of Formula (C-XI)

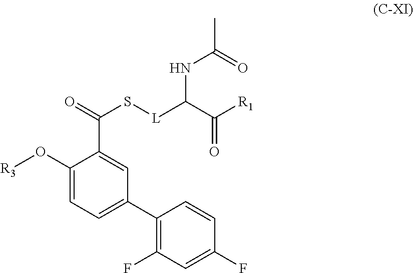

or a pharmaceutically acceptable salt thereof, wherein

R₁ is OR₂, NR₄R₅, or

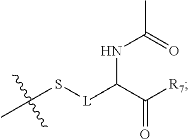

R₂ is (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens;

Z₁ and Z₂ are independently H or (C₁-C₆)alkyl;

R₃ is H or C(O)R₆;

R₄ and R₅ are independently H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₁Z₂, or phenyl, wherein the phenyl is optionally substituted with 1, 2, 3, 4, or 5 halogens; or R₄ and R₅ together with the nitrogen atom to which they are attached form azetidine, pyrrolidine, piperidine, piperazine, N-methylpiperazine, morpholine, or azepane;

R₆ is H, (C₁-C₆)alkyl, (C₃-C₈)cycloalkyl, or (C₃-C₈)cycloalkyl(C₁-C₆)alkyl, wherein the (C₁-C₆)alkyl, (C₃-C₈)cycloalkyl, and (C₃-C₈)cycloalkyl(C₁-C₆)alkyl are optionally substituted with 1, 2, 3, or 4 substituents that are independently (C₁-C₆)alkoxy, (C₁-C₆)alkoxy(C₁-C₆)alkyl, (C₁-C₆)alkoxycarbonyl, (C₁-C₆)alkylthio, halogen, hydroxy, hydroxycarbonyl, NZ₃Z₄, or phenyl, wherein the phenyl is optionally substituted with 1, 2, 3, 4, or 5 halogens;

Z₃ and Z₄ are independently H or (C₁-C₆)alkyl;

R₇ is OR₂ or NR₄R₅; and

L is CH₂CH₂.

### Methods of Treatment

In another aspect, the present invention provides methods for treating atherosclerosis, neuropathy, nephropathy, retinopathy, inflammatory disorders, Chronic Obstructive Pulmonary Disease, cardiovascular diseases, and metabolic disorders in a mammal or patient that comprises administering to the mammal or patient in need of such treatment a therapeutically effective amount of a pharmaceutical combination of the disclosure.

In certain embodiments, the inventive methods include treating dyslipidemia, insulin resistance, elevated free fatty acids, elevated triglycerides, β-cell dysfunction, hyperglycemia, metabolic syndrome, and any form of diabetes mellitus including type I and type II diabetes and Latent Autoimmune Diabetes of Adulthood, in a patient comprising administering to the patient in need of such treatment a therapeutically effective amount of a pharmaceutical combination of the disclosure.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a pharmaceutical combination of the disclosure.

In certain embodiments, the present invention provides methods for treating β-cell dysfunction in a patient comprising administering to the patient in need of such treatment a therapeutically effective amount of a pharmaceutical combination of the disclosure.

In certain embodiments, the present invention provides methods for treating hyperglycemia in a patient comprising administering to the patient in need of such treatment a therapeutically effective amount of a pharmaceutical combination of the disclosure.

In certain embodiments, the present invention provides methods for reducing free fatty acids in a patient comprising administering to the patient in need of such treatment a therapeutically effective amount of a pharmaceutical combination of the disclosure.

In certain embodiments, the present invention provides methods for reducing triglycerides in a patient comprising administering to the patient in need of such treatment a therapeutically effective amount of a pharmaceutical combination of the disclosure.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a pharmaceutical combination of the disclosure.

In certain embodiments, the present invention provides uses for pharmaceutical combinations of the disclosure for preparing, or for the manufacture of, a medicament for treating dyslipidemia, insulin resistance, elevated free fatty acids, elevated triglycerides, β-cell dysfunction, hyperglycemia, metabolic syndrome, and any form of diabetes mellitus including type I and type II diabetes and Latent Autoimmune Diabetes of Adulthood, in a patient.

In certain embodiments, the present invention provides uses for pharmaceutical combinations of the disclosure for preparing, or for the manufacture of, a medicament for reducing advanced glycated end products and/or lipid peroxidation including, but not limited to, oxidation of low-density lipoproteins in a patient.

For each of the pharmaceutical combinations set forth herein, the invention provides in separate aspects provides methods for reducing free fatty acids, triglycerides, advanced glycated end products, ROS, lipid peroxidation, tissue and plasma TNFα and IL6 levels, or for delaying or preventing cardiovascular complications associated with atherosclerosis in a mammal or human patient comprising administering to the mammal or human patient in need of such treatment a therapeutically effective amount of a pharmaceutical combination, as described herein, or a pharmaceutical composition including a pharmaceutical combination of the disclosure.

For each of the combination set forth herein, the invention provides in separate aspects provides methods for protecting pancreatic beta-cells, preventing their impairment or failure and subsequent lower insulin secretion, in a mammal or human patient comprising administering to the mammal or human patient in need of such treatment a therapeutically effective amount of a pharmaceutical combination, as described herein, or a pharmaceutical composition including a pharmaceutical combination of the disclosure.

For each of the combination set forth herein, the invention provides in separate aspects provides uses for pharmaceutical combinations, or pharmaceutical compositions comprising these pharmaceutical combinations, for preparing, or for the manufacture of, a medicament for treating atherosclerosis, neuropathy, nephropathy, retinopathy, inflammatory disorders, COPD, cardiovascular diseases, metabolic disorders, type I diabetes mellitus, type II diabetes mellitus, LADA, metabolic syndrome, dyslipidemia, hyperglycemia, or insulin resistance in a mammal or human patient. The present invention also provides uses for pharmaceutical combinations, or pharmaceutical compositions comprising these pharmaceutical combinations, for preparing, or for the manufacture of, a medicament for reducing free fatty acids, triglycerides, advanced glycated end products, ROS, lipid peroxidation, tissue and plasma TNFα and IL6 levels, or for delaying or preventing cardiovascular complications associated with atherosclerosis in a mammal or human patient. The present invention also provides uses for pharmaceutical combinations, or pharmaceutical compositions comprising these pharmaceutical combinations, for preparing, or for the manufacture of, a medicament for protecting pancreatic β-cells, preventing their impairment or failure and subsequent lower insulin secretion, in a mammal or human patient.

In another aspect, the present invention provides methods for treating adipocyte dysfunction related diseases, carbohydrate metabolism related diseases, vascular diseases, neurodegenerative diseases, cancers, arthritis, osteoarthritis, spondylitis, bone resorption diseases, sepsis, septic shock, chronic pulmonary inflammatory disease, fever, periodontal diseases, ulcerative colitis, pyresis, Alzheimer's disease, Parkinson's diseases, cystic fibrosis, dysfunctions of the immune system, stroke, multiple sclerosis, migraine, pain, inflammatory eye conditions including uveitis, glaucoma and conjunctivitis, degenerative bone or joint conditions including osteoarthritis, rheumatoid arthritis, rheumatoid spondylitis, gouty arthritis ankylosing spondylitis, psoriatic arthritis and other arthritic conditions, as well as inflamed joints, chronic inflammatory skin conditions, including allergic lesions, lichen planus, pityriasis rosea, eczema, psoriasis, and dermatitis, diseases and disorders of the gastrointestinal tract, including inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections, for example, by Helicobacter pylori, inflammatory lung disorders such as asthma, bronchitis, particularly chronic obstructive pulmonary disease, farmer's lung, acute respiratory distress syndrome; bacteraemia, endotoxaemia (septic shock), aphthous ulcers, gingivitis, pyresis, particularly pain, including inflammatory pain, neuropathic pain, acute pain or pain of a central origin; meningitis and pancreatitis, and other conditions associated with inflammation, central nervous system inflammatory conditions and diseases, including ischaemia-reperfusion injury associated with ischemic stroke; vascular diseases, such as atheromatous and nonatheromatous, ischemic heart disease, and Raynaud's Disease and Phenomenon in a mammal or patient comprising administering to the mammal or patient in need of such treatment a therapeutically effective amount of a pharmaceutical combination of the disclosure. In certain embodiments, the present invention provides uses for the pharmaceutical combination of the disclosure for preparing, or for the manufacture of, a medicament for treating the diseases/disorders listed above.

In another aspect, the present invention provides methods for treating adipocyte dysfunction related diseases, carbohydrate metabolism related diseases, vascular diseases, neurodegenerative diseases, cancers, arthritis, osteoarthritis, spondylitis, bone resorption diseases, sepsis, septic shock, chronic pulmonary inflammatory disease, fever, periodontal diseases, ulcerative colitis, pyresis, Alzheimer's disease, Parkinson's diseases, cystic fibrosis, dysfunctions of the immune system, stroke, multiple sclerosis, migraine, pain, inflammatory eye conditions including uveitis, glaucoma and conjunctivitis, degenerative bone or joint conditions including osteoarthritis, rheumatoid arthritis, rheumatoid spondylitis, gouty arthritis ankylosing spondylitis, psoriatic arthritis and other arthritic conditions, as well as inflamed joints, chronic inflammatory skin conditions, including allergic lesions, lichen planus, pityriasis rosea, eczema, psoriasis, and dermatitis, diseases and disorders of the gastrointestinal tract, including inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections, for example, by Helicobacter pylori, inflammatory lung disorders such as asthma, bronchitis, particularly chronic obstructive pulmonary disease, farmer's lung, acute respiratory distress syndrome; bacteraemia, endotoxaemia (septic shock), aphthous ulcers, gingivitis, pyresis, particularly pain, including inflammatory pain, neuropathic pain, acute pain or pain of a central origin; meningitis and pancreatitis, and other conditions associated with inflammation, central nervous system inflammatory conditions and diseases, including ischaemia-reperfusion injury associated with ischemic stroke; vascular diseases, such as atheromatous and nonatheromatous, ischemic heart disease, and Raynaud's Disease and Phenomenon in a patient comprising administering to the patient in need of such treatment a therapeutically effective amount of a pharmaceutical composition comprising at least one pharmaceutically acceptable carrier and a pharmaceutical combination of the disclosure. In certain embodiments, the present invention provides uses for pharmaceutical compositions for preparing, or for the manufacture of, a medicament for treating the diseases/disorders listed above, wherein the pharmaceutical composition comprises at least one pharmaceutically acceptable carrier and a pharmaceutical combination of the disclosure.

### Pharmaceutical Compositions

In another aspect, the present invention provides pharmaceutical combinations of the disclosure, as described above, and at least one pharmaceutically acceptable carrier.

The term “pharmaceutically acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration.

The pharmaceutical compositions of this invention can be administered to humans (patients) and other mammals orally, rectally, parenterally, intracisternally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more pharmaceutically acceptable carriers as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, free fatty acids such as oleic acid are used in the preparation of injectables.

Solid dosage forms for oral administration include capsules, tablets, pills, powders, and granules. In such solid dosage forms, the active compound is mixed with at least one inert pharmaceutically acceptable carrier such as sodium citrate or calcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and free fatty acid esters of sorbitan, and mixtures thereof

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

The phrase “therapeutically effective amount” of the compound of the present invention means a sufficient amount of the compound to treat metabolic disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Actual dosage levels of active ingredients in the pharmaceutical compositions of this invention can be varied so as to obtain an amount of the active compound(s) which is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated.

In certain embodiments, the total daily dose of the compounds of this invention administered to a mammal, and particularly a human, are in the range of from about 0.03 to about 20 mg/kg/day. For purposes of oral administration, more preferable doses can be in the range of from about 0.1 to about 10 mg/kg/day. If desired, the effective daily dose can be divided into multiple doses for purposes of administration, e.g. two to four separate doses per day.

The term “pharmaceutically acceptable salt,” as used herein, means a positively-charged inorganic or organic cation that is generally considered suitable for human consumption. Examples of pharmaceutically acceptable cations are alkali metals (lithium, sodium and potassium), magnesium, calcium, ferrous, ferric, ammonium, alkylammonium, dialkylammonium, trialkylammonium, tetraalkylammonium, diethanolammmonium, and choline. Cations may be interchanged by methods known in the art, such as ion exchange. Where compounds of the present invention are prepared in the carboxylic acid form, addition of a base (such as a hydroxide or a free amine such as an alpha amino acid) will yield the appropriate salt form, (L) lysine is a preferred free amine for preparing salts of the present invention.

The present invention contemplates pharmaceutically active metabolites formed by in vivo biotransformation of combinations of the disclosure. The term pharmaceutically active metabolite, as used herein, means a compound formed by the in vivo biotransformation of the combinations. A thorough discussion of biotransformation is provided in (Goodman and Gilman's, The Pharmacological Basis of Therapeutics, seventh edition).

### EXAMPLES

The embodiments of the invention are further by the following examples, which are not to be construed as limiting the disclosure in scope or spirit.

### Example 1

A combination of metformin and compound GMC-252 is prepared. Metformin HCl is dosed at 200/300 mg/kg/day, and GMC-252 lysine salt is dosed at 0.2 mmol/kg/day. Compound GMC-252, which has the following structure:

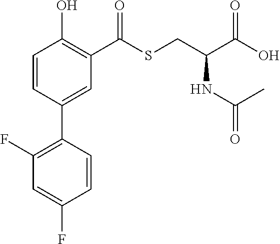 [SMILES: CC(=O)N[C@@H](CSC(=O)c1cc(-c2ccc(F)cc2F)ccc1O)C(=O)O]

is disclosed in WO/2010/106082 and is described below as (Example A-13).

### Example 2

A combination of exenatide and compound GMC-252 is prepared. Exenatide is dosed at 0.25 nmol/kg/Day, and GMC-252 lysine salt is dosed at 0.2 mmol/kg/day.

### Example 3

A combination of exenatide and compound GMC-252 is prepared. Exenatide is dosed at 2.5 nmol/kg/Day, and GMC-252 lysine salt is dosed at 0.2 mmol/kg/day.

### Methods

### Pharmacokinetic Studies:

Male cd-1 mice weighing 25-30 g are purchased from Charles River Laboratories Spain. The animals are housed in animal quarters at 22° C. with a 12-h light/12-h dark cycle and fed ad libitum. O/N fasted animals are dosed at 9:00 pm with 0.05 mmol/kg of the indicated combination. Mice are sacrificed at the indicated time points, with CO₂ euthanasia, and blood is extracted from the inferior cava vein, using heparin as an anticoagulant, and maintained at 4° C. until the preparation of plasma. Plasma was separated after centrifugation of blood and kept at −20° C. until metabolites determination.

### In Vitro Cleavage Studies:

The combinations are incubated with human liver S9 fraction to study metabolic stability and to profile and identify the forming metabolites. The basic incubation mixture of 500 μl in volume consisted of the following components: 1.5 mg of protein per ml, substrate in DMSO, 1 mM NADPH, 1 mM UDPGA, 1 mM PAPS and 1 mM GSH. The substrate concentration used was 2 μM. The final amount of DMSO in the incubation was 1% (v/v). Each reaction mixture was preincubated for 2 minutes at +37° C. The reaction was started by addition of cofactors. After an incubation of 60 min, a 100 μl sample was collected and the reaction was terminated by adding an equal volume of ice-cold acetonitrile. Samples were subsequently cooled in an ice bath for 15 minutes and analyzed. The incubation samples were thawed at room temperature (RT), shaken and centrifuged for 10 min at 16100×g and pipetted to Maximum Recovery vials (Waters Corporation, Milford, Mass., USA). The samples are analyzed by UPLC/TOF-MS to monitor both the disappearance of the parent combination and formation of metabolites. The analytical method is optimised by using the parent compounds for fit-for-purpose chromatographic properties (peak shape and retention) and mass spectrometric ionisation. In addition to ion source conditions optimised to produce molecular ions with high abundance, the samples from last time point (60 min) are analysed also using another parameters (higher aperture voltage) to generate high-resolution in-source fragment ion data.

Parent disappearance is estimated based on relative LC/MS peak areas. The metabolites are mined from the data acquired from the 60 min point samples and the detected metabolites (biotransformations) are tentatively identified according to the accurate MS-data obtained. For the main metabolites also the in-source fragment ion data is used for elucidating the biotransformation sites.

### Chemicals.

The chemicals were purchased from Sigma (Sigma Aldrich, St. Louis, Mo., USA) and PBS was purchase from Invitrogen. All the compounds were dissolved in PBS, with lysine salt when indicated, and the pH of the compounds without lysine was adjusted with NaOH 6N until pH 7.

Chronic Treatment in db/db Mice, ob/ob Mice and Zucker Diabetic Rats

5-weeks old male mice C57BL/Ks bearing the db/db mutation (The Jackson Laboratories) and 7-weeks old male mice C57BL/6 bearing the ob/ob mutation are purchased from Charles River Laboratories Spain (Sant Cugat del Valles, Spain) are treated with combinations as disclosed herein for a month, administered by single oral injection. Glycemia levels are determined in blood from the Tail Vein, using a rapid glucose analyzer (Accu-Chek Aviva; Roche) 3 times per week, as well as body weight measure. The food and water intake is measured twice a week. At the end of the treatment, the mice are sacrificed, in feeding state, with CO₂ euthanasia, and the blood extracted from the Inferior Cave Vein, using heparin as an anticoagulant, and maintained at 4° C. until the preparation of plasma.

Intraperitoneal Insulin Tolerance Test (ipITT).

At the third week of treatment, an Insulin Tolerance Test (ITT) is performed as follows. Animals receive an ip injection of Insulin 2 UI/kg (Humulin®) and glycemia levels are determined at time zero (before the injection of insulin) and at different time points thereafter in blood from the Tail Vein, after the Insulin injection using a rapid glucose analyzer (Accu-Chek Aviva; Roche).

Intraperitoneal Glucose Tolerance Test (ipGTT)

At the fourth week of treatment, a Glucose Tolerance Test (GTT) is performed as follows. Overnight fasted animals receive an ip injection of Glucose 0.5 g/kg (Glucosmon 50®). Glycemic levels are determined, at time zero (before the injection of glucose) and at different time points thereafter, in blood from the Tail Vein using a rapid glucose analyzer (Accu-Chek Aviva; Roche).

### Determination of Biochemical Parameters.

The circulating glucose concentration is determined by a rapid glucose analyzer

(Accu-Chek Aviva; Roche). Plasma triglycerides and non esterified fatty acids are determined using conventional colorimetric methods (commercially available from Biosystems, Barcelona, Spain, and Wako Chemicals, Neuss, Germany, respectively). Plasma insulin concentration is determined by enzyme-linked immunosorbent assay method (CrystalChem, Downers Grove, Ill.).

### Statistical Analysis.

Statistical comparisons between groups are established by two-way ANOVA using Prism 4 (GraphPad, San Diego, Calif.). A p value of less than 0.05 is considered to be statistically significant.

Reduction of Fasting Glycemia in db/db Mice

db/db mice were treated for two weeks with a daily dose of the c. A t-test indicated that the value of p was less than 0.01.

### Protection of β-Cell Failure and Prevention of Hyperglycemia in Streptozotocin Treated Animals

Diabetic mice or rats generated by streptozotocin administration exhibit an increase in levels of lipid peroxidation and a decrease in activity of antioxidant enzymes in the liver and kidneys as compared to control.

Conjugates of the invention administered orally and/or intraperitoneally (˜250 mg/kg) prior to a single dose of streptozotocin (45 mg/kg i.p.) in rats followed by 4 additional treatment days can preserve β-cells, reducing the development of hyperglycemia. The blood glucose level in pretreated animals is lower than the control group associated with a preserve capacity of β-cell to secrete insulin measured in the blood.

Further, combinations of the invention are tested for their efficiency at preserving β-cell function of mice challenged by one shot of streptozotocin (45 mg/kg i.p.). Oral or intraperitoneal administration of a conjugate of the invention, prior and during 5 days following streptozotocin exposure can protect β-cells from oxidative stress and reduces the development of hyperglycemia over time compared to control.

Combinations of the invention can reduce levels of 8-hydroxy-deoxyguanosine (8OhdG) and malondialdehyde+4-hydroxy-2-nonenal (4HNE), markers for both oxidative stress and lipid peroxidation in the blood.

1. Type 1 Diabetic Model in Mice

Diabetic mice induced by streptozotocin injection (120 mg/kg i.p.) are treated for 4 weeks with 250 mg/kg/day (oral or i.p.) of a combination of the invention. At the end of the 4 week treatment, fasting glucose, fructosamine, triglycerides and cholesterol are measured. These biochemical parameters can be reduced in comparison to control group.

Further, oxidative stress and lipid peroxidation markers 8-hydroxy-deoxyguanosine (8OhdG), malondialdehyde and 4-hydroxy-2-nonenal (4HNE) are also reduced.

Still further, inflammatory cytokines, such as TNFα and IL-6, and glutathione (GSH) levels in the liver and the kidney can be reduced compared to non-treated animals.

### In Vivo Beta Cell Protection Model

Beta-cell destruction is induced in cd-1 mice after 3 hours of fasting by a single intraperitoneal injection of a freshly prepared solution of alloxan 200 mg/kg (Sigma-Aldrich, San Luis, Mo.) that was dissolved in 0.9% NaCl. Combinations of the invention or vehicle control are administered intraperitoneally, 1 hour before alloxan administration. At the end of the treatment, at day 4, animals are killed and the plasma collected and kept at −20° C. until used. Conjugates of Formula as disclosed herein can beneficially reduce plasma glucose levels in Alloxan-treated animals as compared to control animals.

Restoration of Insulin Sensibility in ob/ob and db/db Mice

5-8 week old ob/ob and db/db mice are treated for 3 to 4 weeks with a daily dose of 50 to 250 mg/kg of a combination of the invention by oral gavage or with drug mix with food or subcutaneously.

Glucose tolerance test (OGTT or IPTT) can detect reduction in glucose level elevation during the test compared to non-treated animals. The capacity of the β-cells to secrete insulin can be improved in the group administered with a combination of the invention compared to control demonstrating the protective effects toward pancreatic β-cells.

Further, combinations of the invention can improve insulin sensitivity as evidenced by a sustained and pronounced glucose lowering effect. Also, the combinations of the invention can provide reduction in oxidative stress and lipid peroxidation as determined by the level of associated biomarkers: 8-hydroxy-deoxyguanosine (8OhdG), malondialdehyde and 4-hydroxy-2-nonenal (4HNE). Finally, inflammatory cytokines, TNFα and IL-6, can be reduced while the levels of glutathione (GSH) in the liver and the kidney are restored.

### Restoration of Insulin Sensitivity in Zucker Diabetic Fatty (ZDF) Rats

To assess whether conjugates comprising an antioxidant agent and an inflammatory agent would prevent glucose toxicity and progression of diabetes mellitus associated with β-cell failure overtime, the capacity of conjugates of Formula (I, II and III) to alter development of disease in this Type 2 diabetic animal model is assessed.

Zucker diabetic rats from 6 to 12 weeks of age are treated daily with an oral dose of a combination of the invention. Blood levels of 8OhdG, malondialdehyde+4HNE, two markers of chronic oxidative stress and lipid peroxidation, can be reduced in comparison to control animals. Inflammatory cytokines, TNFα and IL6 can be blunted when measured at the end of the 6 week treatment. In comparison, placebo-treated or control animals can develop progressive obesity, hyperglycemia, abnormal glucose tolerance test, defective glucose insulin secretion as well decrease islet insulin content. Further, treatment with combinations of the invention can at least partially prevent worsening of hyperglycemia, improve results of the glucose tolerance test, and preserve insulin secretion from β-cells. Fasting glucose, fructosamine, Hb1Ac, triglycerides and cholesterol all can be reduced in comparison to the control group.

### Results

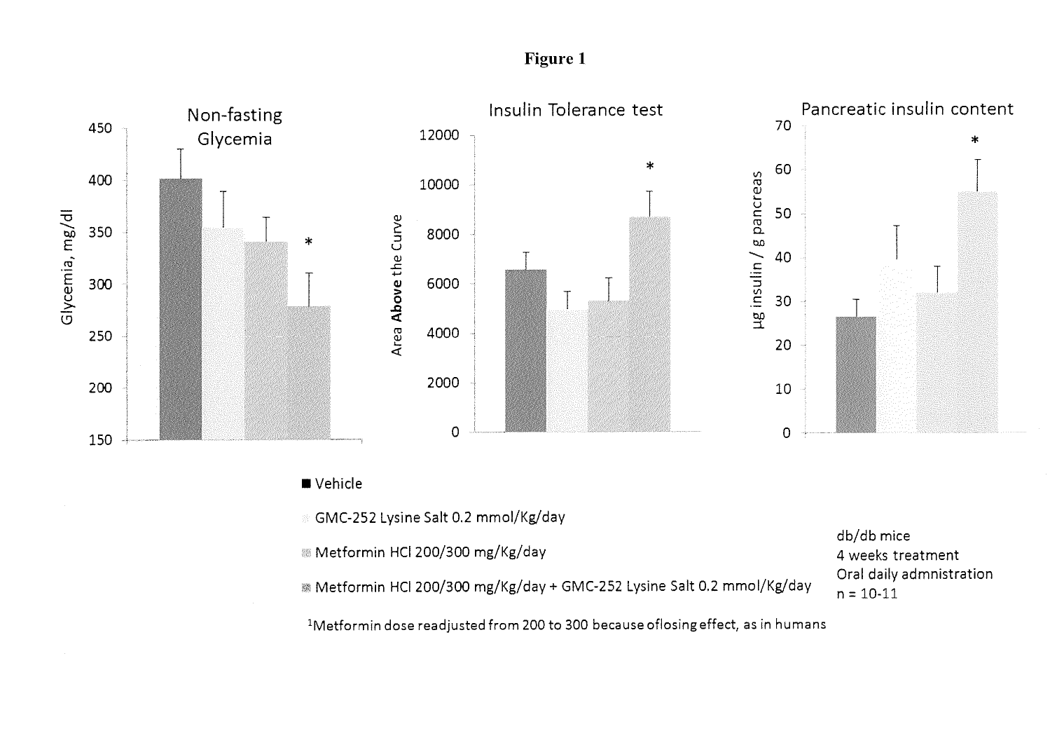

The combination described in Example 1 was evaluated orally in mice for non-fasting glycemia, intraperitoneal insulin tolerance (ipITT) and pancreatic insulin content (FIG. 1).

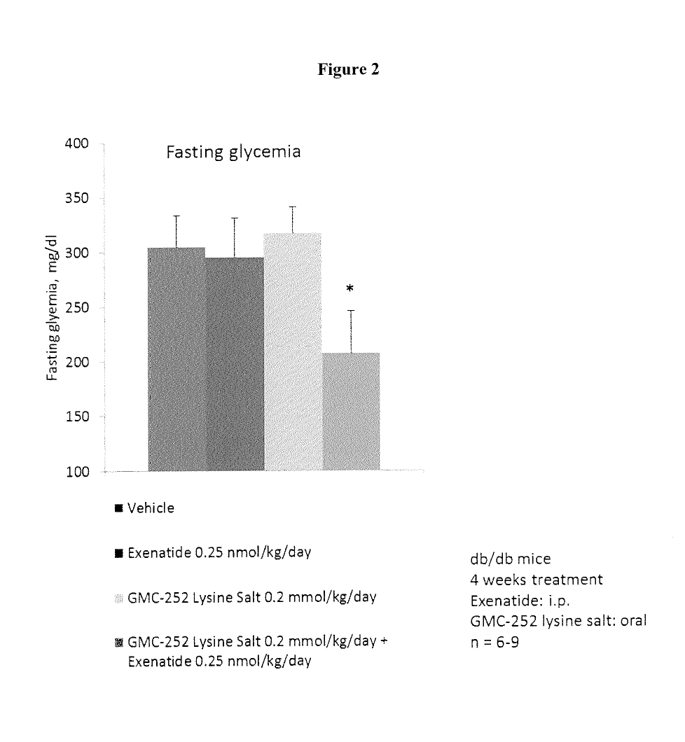

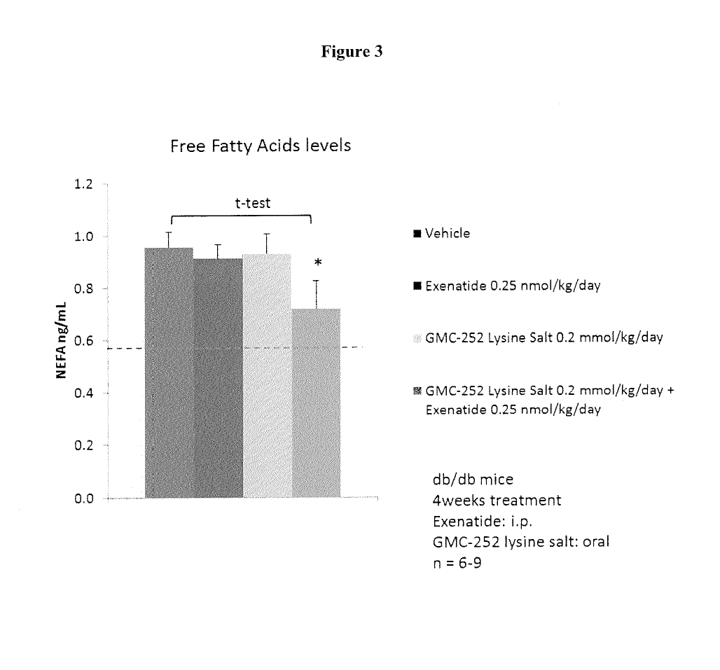

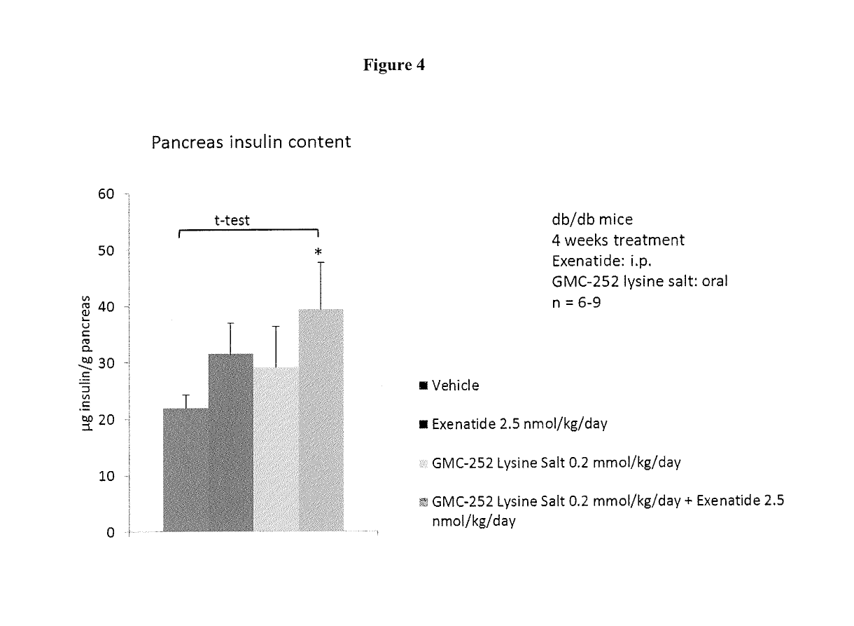

The combination described in Example 2 was evaluated orally in mice for the fasting glycemia (FIG. 2) and the level of non-esterified fatty acids (NEFA) (FIG. 3). The combination described in Example 3 was evaluated orally in mice for the pancreas insulin level (FIG. 4)

Combinations of the invention can have beneficial effects in Type 2 diabetic animal models as compared to control animals, including hypolipidemic and anti-diabetic effects as well as antioxidant properties in different animal models of diabetes useful in preventing the development of β-cell failure and aggravation of the diabetic status leading to cardiovascular complications. Such effects support therapeutic utility of the combinations of the invention.

Moreover the additive and/or synergistic effects of these combinations allow for the decreased dosing of each independent active ingredient. These additive and/or synergistic effects reduce the liability of side effects associated with a salicylate agent, gastric bleeding, or an antioxidant, tinnitus, given to a patient alone.

### Compounds

### Example A-1

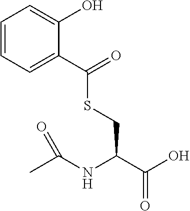 [SMILES: CC(=O)N[C@@H](CSC(=O)c1ccccc1O)C(=O)O]

### Salnacedin (R)-2-acetamido-3-(2-hydroxybenzoylthio)propanoic acid

The title compound is prepared using the procedures described in EP 0 080 229.

### Example A-2

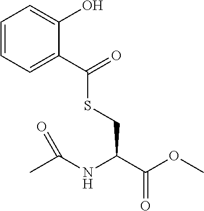 [SMILES: COC(=O)[C@H](CSC(=O)c1ccccc1O)NC(C)=O]

### (R)-methyl 2-acetamido-3-(2-hydroxybenzoylthio)propanoate

The title compound is prepared using similar procedures as described in EP 0 080 229.

### Example A-3

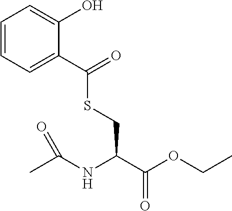 [SMILES: CCOC(=O)[C@H](CSC(=O)c1ccccc1O)NC(C)=O]

### (R)-ethyl 2-acetamido-3-(2-hydroxybenzoylthio)propanoate

The title compound is prepared using similar procedures as described in EP 0 080 22929.

### Example A-4

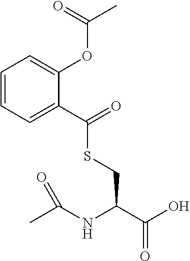 [SMILES: CC(=O)N[C@@H](CSC(=O)c1ccccc1OC(C)=O)C(=O)O]

### (R)-2-acetamido-3-(2-acetoxybenzoylthio)propanoic acid

The title compound is prepared using similar procedures as described in EP 0 080 229.

### Example A-5

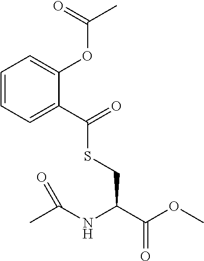 [SMILES: COC(=O)[C@H](CSC(=O)c1ccccc1OC(C)=O)NC(C)=O]

### (R)-methyl 2-acetamido-3-(2-acetoxybenzoylthio)propanoate

The title compound is prepared using similar procedures as described in EP 0 080 229.

### Example A-6

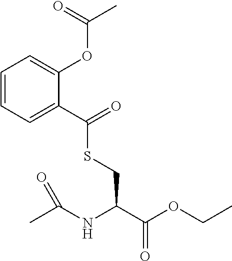 [SMILES: CCOC(=O)[C@H](CSC(=O)c1ccccc1OC(C)=O)NC(C)=O]

### (R)-ethyl 2-acetamido-3-(2-acetoxybenzoylthio)propanoate

The title compound is prepared using similar procedures as described in EP 0 080 229.

### Example A-7

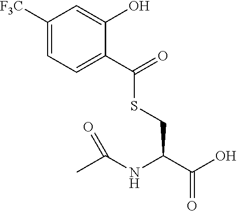 [SMILES: CC(=O)N[C@@H](CSC(=O)c1ccc(C(F)(F)F)cc1O)C(=O)O]

### (R)-2-acetamido-3-(2-hydroxy-4-(trifluoromethyl)benzoylthio)propanoic acid

The title compound is prepared using similar procedures as described in EP 0 080 229.

### Example A-8

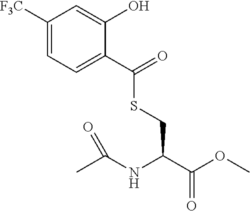 [SMILES: COC(=O)[C@H](CSC(=O)c1ccc(C(F)(F)F)cc1O)NC(C)=O]

### (R)-methyl 2-acetamido-3-(2-hydroxy-4-(trifluoromethyl)benzoylthio)propanoate

The title compound is prepared using similar procedures as described in EP 0 080 229.

### Example A-9

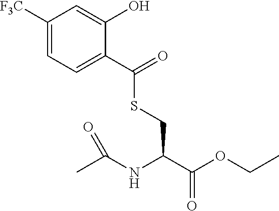 [SMILES: CCOC(=O)[C@H](CSC(=O)c1ccc(C(F)(F)F)cc1O)NC(C)=O]

### (R)-ethyl 2-acetamido-3-(2-hydroxy-4-(trifluoromethyl)benzoylthio)propanoate

The title compound is prepared using similar procedures as described in EP 0 080 229.

### Example A-10

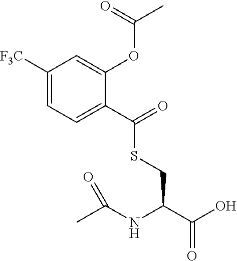 [SMILES: CC(=O)N[C@@H](CSC(=O)c1ccc(C(F)(F)F)cc1OC(C)=O)C(=O)O]

### (R)-2-acetamido-3-(2-acetoxy-4-(trifluoromethyl)benzoylthio)propanoic acid

The title compound is prepared using similar procedures as described in EP 0 080 229.

### Example A-11

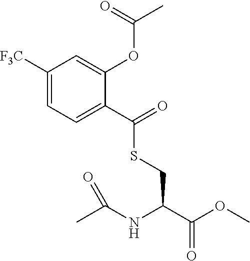 [SMILES: COC(=O)[C@H](CSC(=O)c1ccc(C(F)(F)F)cc1OC(C)=O)NC(C)=O]

### (R)-methyl 2-acetamido-3-(2-acetoxy-4-(trifluoromethyl)benzoylthio)propanoate

The title compound is prepared using similar procedures as described in EP 0 080 229.

### Example A-12

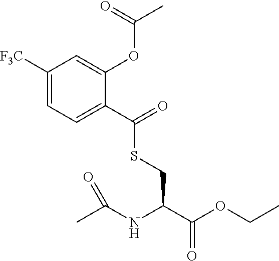 [SMILES: CCOC(=O)[C@H](CSC(=O)c1ccc(C(F)(F)F)cc1OC(C)=O)NC(C)=O]

### (R)-ethyl 2-acetamido-3-(2-acetoxy-4-(trifluoromethyl)benzoylthio)propanoate

The title compound is prepared using similar procedures as described in EP 0 080 229.

### Example A-13

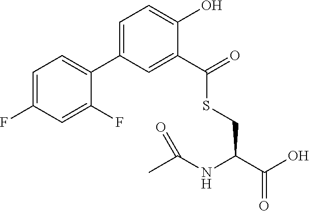 [SMILES: CC(=O)N[C@@H](CSC(=O)c1cc(-c2ccc(F)cc2F)ccc1O)C(=O)O]

### R)-2-acetamido-3-(2′,4′-difluoro-4-hydroxybiphenylcarbonylthio)propanoic acid (GMC-252

The title compound is prepared using the procedures described in BE 900328. The title compound was also commercially available. However, alternatively the compound was synthesized as follows.

### Example A-13a

To a solution of 2′,4′-difluoro-4-hydroxy-1,1′-diphenyl-3-carboxylic acid (Diflunisal, 82.5 g, 0.329 mol) dissolved in acetone (450 mL) and cooled to −10° C. (refrigerant mixture: ice-EtOH) was added Et₃N (101 mL, 0.725 mol) slowly (addition: 25 min, internal temperature: from −8° C. to 9° C.). To the resulting solution was added 2,2,2-trichloroethyl chloroformate (100 mL, 0.725 mol) slowly (addition: 60 min, internal temperature was maintained below 0° C.: from −10° C. to 0° C.). The mixture was stirred for 1 h at 0° C. (a white precipitate of triethylamine hydrochloride was gradually formed). At the end of the reaction, the mixture was filtered under vacuum, the precipitate (triethylamine hydrochloride) was washed with acetone (4×180 mL) and the filtrate was evaporated under vacuum at 30° C. The oily residue was taken with Et₂O (150 mL) and the suspension was evaporated again under vacuum. The operation was repeated three times to remove excess of chlorocarbonate. The residue was dissolved in acetone (180 mL), and added to a refrigerated solution of N-acetyl-L-cysteine (N-Ac-Cys, 53.81 g, 0.329 mol) and Et₃N (46 mL, 0.329 mol) in acetone (140 mL) slowly (addition: 55 min, internal temperature was maintained below 15° C.: from 0° C. to 15° C.). The reaction mixture was stirred at 15° C. for 4 h. The mixture was cooled to −12° C. (internal temperature), and Et₃N (115 mL, 0.824 mol) was added. The mixture was stirred for 15 h at −12° C. (internal temperature), and at the end of the reaction, the mixture was filtered under vacuum and the precipitate was washed with acetone (3×150 mL). The oily precipitate was suspended in CH₂Cl₂ (400 mL), cooled to 0° C. and an aqueous HCl solution (15% v:v) was added with vigorous stirring until the pH was lowered to 3. Ethanol (80 mL) was added, and the aqueous phase was extracted with CH₂Cl₂ (2×400 mL). The combined organic layers were washed with a 10% HCl aqueous solution (1×500 mL) and with water (2×600 mL), were dried over anhydrous Na₂SO₄, filtered and concentrated. The residue was purified by trituration with Et₂O (100 mL), affording 44.13 g of the title compound (HPLC purity: 88.26%) To increase purity, the solid was suspended in Et₂O (100 mL) and stirred at room temperature for 20 min. The solid was filtered under vacuum and was washed with Et₂O (3×100 mL), to afford 31.33 g of the title compound GMC-252 (Rf=0.3 CH₂Cl₂/MeOH/AcOH 95:5:1), white solid, 24% yield, 96.22% HPLC purity); Purity was determined by NMR analysis and mass spectrometry to conform to the following parameters: 1H-NMR (CD₃OD, 250 MHz, δ): 8.00 (m, 1H, ArH); 7.66 (dm, J=8.2 Hz, 1H, ArH); 7.50 (m, 1H, ArH); 7.06 (m, 3H, ArH); 4.74 (m, 1H, CH); 3.77 (dd, J=4.7 and 13.7 Hz, 1H, CH); 3.40 (m, 1H, CH); 1.98 (s, 3H, CH₃); MS-EI+m/z: 396.00 (M+1); LC-MS: M+1: 396.00; purity: 96.52% (HPLC method: SunFire C18 3.5 um, 2.1×100 mm, flow: 0.3 mL/min, gradient: A:B 3 min 10:90+from 10:90 to 95:5 in 17 min+10 min 95:5; A: CH₃CN:MeOH 1:1; B: NH₄OAc buffer 5 mM pH 7).

### Example A-13b

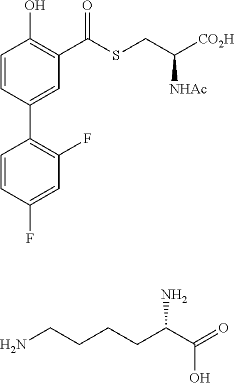 [SMILES: CC(=O)N[C@@H](CSC(=O)c1cc(-c2ccc(F)cc2F)ccc1O)C(=O)O ; NCCCC[C@H](N)C(=O)O]

### 2R)-2-(Acetylamino)-3-{[(2′,4′-difluoro-4-hydroxy-1,1′-biphenyl-3-yl)carbonyl]thio}propanoic acid L-Lysine Salt (GMC-252-L-Lysine salt

Starting material (GMC-252, 18.33 g, 46.37 mmol, Example A-13a) was dissolved in acetone (300 mL) and L-Lysine (L-Lys, 6.44 g, 44.05 mmol) dissolved in H₂O (60 mL) was added. Acetone (100 mL) was added and the mixture was stirred at room temperature for 1 h. The resulting solid was filtered under vacuum, washed with acetone (3×100 mL), Et₂O (2×80 mL), and hexanes (2×80 mL). The solid was dried at room temperature to give 22.01 g of title salt GMC-252.L-Lys as a white solid. (92% yield, 99.59% HPLC purity). ¹H-NMR (D₂O, 250 MHz) 6 7.77 (m, 1H, ArH); 7.50 (d, J=8.5 Hz, 1H, ArH); 7.23 (m, 1H, ArH); 6.90 (m, 3H, ArH); 4.47 (m, 1H, CH); 3.73-3.59 (m, 2H, CH); 3.25 (m, 1H, CH); 2.97 (t, J=7.4 Hz, 2H, CH₂); 1.94 (s, 3H, CH₃);); 1.84 (m, 2H, CH₂); 1.67 (m, 2H, CH₂); 1.44 (m, 2H, CH₂); MS-EI⁺ m/z: 396.00 (M+1-L-Lys); LC-MS: M+11-L-Lys: 396.00; purity: 99.59% (HPLC method: SunFire C18 3.5 um, 2.1×100 mm, flow: 0.3 mL/min, gradient: A:B 3 min 10:90+from 10:90 to 95:5 in 17 min+10 min 95:5; A: CH₃CN:MeOH 1:1; B: NH₄OAc buffer 5 mM pH 7).

### Example A-14

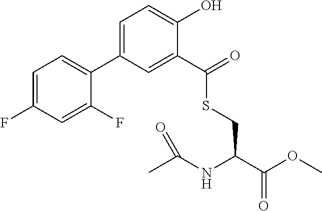 [SMILES: COC(=O)[C@H](CSC(=O)c1cc(-c2ccc(F)cc2F)ccc1O)NC(C)=O]

### (R)-methyl 2-acetamido-3-(2′,4′-difluoro-4-hydroxybiphenylcarbonylthio)propanoate

The title compound is prepared using similar procedures as described in BE 900328.

### Example A-15

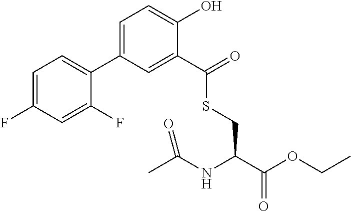 [SMILES: CCOC(=O)[C@H](CSC(=O)c1cc(-c2ccc(F)cc2F)ccc1O)NC(C)=O]

### (R)-ethyl 2-acetamido-3-(2′,4′-difluoro-4-hydroxybiphenylcarbonylthio)propanoate

The title compound is prepared using similar procedures as described in BE 900328.

### Example A-16

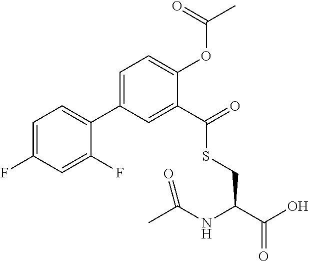 [SMILES: CC(=O)N[C@@H](CSC(=O)c1cc(-c2ccc(F)cc2F)ccc1OC(C)=O)C(=O)O]

### (R)-2-acetamido-3-(4-acetoxy-2′,4′-difluorobiphenylcarbonylthio)propanoic acid

The title compound is prepared using similar procedures as described in BE 900328.

### Example A-17

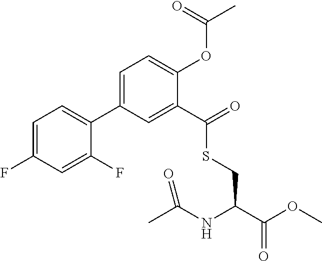 [SMILES: COC(=O)[C@H](CSC(=O)c1cc(-c2ccc(F)cc2F)ccc1OC(C)=O)NC(C)=O]

### (R)-methyl 2-acetamido-3-(4-acetoxy-2′,4′-difluorobiphenylcarbonylthio)propanoate

The title compound is prepared using similar procedures as described in BE 900328.

### Example A-18

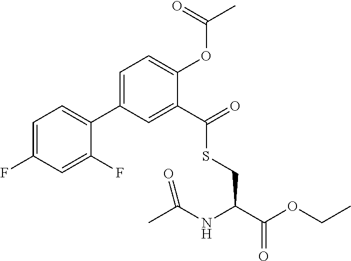 [SMILES: CCOC(=O)[C@H](CSC(=O)c1cc(-c2ccc(F)cc2F)ccc1OC(C)=O)NC(C)=O]

### (R)-ethyl 2-acetamido-3-(4-acetoxy-2′,4′-difluorobiphenylcarbonylthio)propanoate

The title compound is prepared using similar procedures as described in BE 900328.

### Example A-19

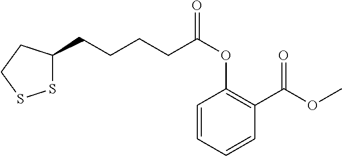 [SMILES: COC(=O)c1ccccc1OC(=O)CCCC[C@@H]1CCSS1]

### Methyl 2-(5-((R)-1,2-dithiolan-3-yl)pentanoyloxy)benzoate

Lipoyl chloride (commercially available, 300 mg) was added slowly to a solution of Methyl 2-hydroxybenzoate (commercially available, 260 mg) and triethylamine (300 mg) in dichloromethane. The reaction was stirred at room temperature for 12 h. The reaction was then concentrated and the residue purified by column chromatography to obtain the desired compound (160 mg) as a pale yellow solid. ¹H-NMR (DMSO) δ: 1.29 (m, 2H); 1.55 (m, 4H); 1.80 (m, 4H); 2.23 (m, 2H); 2.58 (m, 3H); 3.80 (s, 3H); 7.18 (m, 2H); 7.44 (m, 1H); 7.94 (m, 1H).

### Example A-20

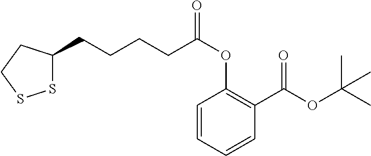 [SMILES: CC(C)(C)OC(=O)c1ccccc1OC(=O)CCCC[C@@H]1CCSS1]

### tert-Butyl 2-(5-((R)-1,2-dithiolan-3-yl)pentanoyloxy)benzoate

The title compound was prepared in a similar manner as that described in Example 19 except using tert-butyl 2-hydroxybenzoate instead of methyl 2-hydroxybenzoate. ¹H-NMR (DMSO) δ: 1.29 (m, 2H); 1.40 (s, 9H); 1.55 (m, 4H); 1.80 (m, 4H); 2.23 (m, 2H); 2.58 (m, 3H); 7.18 (m, 2H); 7.44 (m, 1H); 7.94 (m, 1H).

### Example A-21

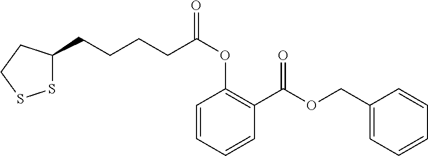 [SMILES: O=C(CCCC[C@@H]1CCSS1)Oc1ccccc1C(=O)OCc1ccccc1]

### Benzyl 2-(5-((R)-1,2-dithiolan-3-yl)pentanoyloxy)benzoate

The title compound was prepared in a similar manner as that described in Example 19 except using benzyl 2-hydroxybenzoate instead of methyl 2-hydroxybenzoate. ¹H-NMR (DMSO) δ: 1.29 (m, 2H); 1.55 (m, 4H); 1.80 (m, 4H); 2.23 (m, 2H); 2.58 (m, 3H); 5.51 (m, 2H); 7.18 (m, 7H); 7.44 (m, 1H); 7.94 (m, 1H).

### Example B-1

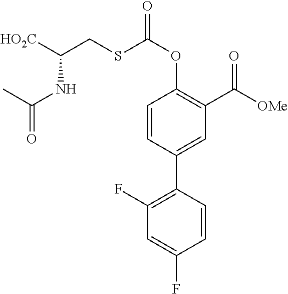 [SMILES: COC(=O)c1cc(-c2ccc(F)cc2F)ccc1OC(=O)SC[C@H](NC(C)=O)C(=O)O]

### (R)-2-Acetamido-3-((2′,4′-difluoro-3-(methoxycarbonyl)biphenyl-4-yloxy) carbonylthio)propanoic acid

### Step 1: (Methyl 2′,4′-difluoro-4-hydroxybiphenyl)-3-carboxylate

H₂SO₄ (9.6 mL, 179.12 mmol) was added to a solution of diflunisal (15.0 g, 59.95 mmol) in MeOH (200 mL). The reaction mixture was refluxed for 12 h and it was allowed to reach r.t. Solids were collected by filtration and washed with cold MeOH (20 mL) to furnish 13.42 g of methyl 2′,4′-difluoro-4-hydroxybiphenyl-3-carboxylate (white solid, yield: 85%).

¹H NMR (CDCl₃, 250 MHz) δ ppm: 10.84 (s, 1H), 7.98 (s, 1H), 7.60 (d, J=8.4 Hz, 1H), 7.36 (c, J₁=8.8 Hz, J₂=6.8 Hz, 1H), 7.06 (d, J=8.8 Hz, 1H), 6.92 (m, 2H), 3.97 (s, 3H).

### Step 2: (R)-2-Acetamido-3-((2′,4′-difluoro-3-(methoxycarbonyl)biphenyl-4-yloxy) carbonylthio)propanoic acid (Method A)

4-Nitrophenyl chloroformate (300 mg, 1.488 mmol) was added to a solution of methyl 2′,4′-difluoro-4-hydroxybiphenyl-3-carboxylate (600 mg, 2.27 mmol) and Et₃N (0.5 mL, 3.587 mmol) in CH₂Cl₂ (20 mL). The reaction mixture was refluxed for 4 h and allowed to reach r.t. It was diluted with CH₂Cl₂ (70 mL) and washed with NaHCO₃ (saturated aqueous solution, 100 mL). The organic layer was dried over Na₂SO₄ (anhydrous), filtered and concentrated. The crude residue was submitted to next step without further purification. The crude residue from previous step was dissolved in DMF (16 mL) and NAC (360 mg, 2.20 mmol) was added. The reaction mixture was stirred at r.t. for 15 min and Et₃N (1.0 mL, 7.11 mmol) was added. The reaction was stirred at r.t. overnight (16 h). It was poured into H₂O (50 mL), taken up to pH=3 by adding HCl (5% aqueous solution) and it was extracted with CH₂Cl₂ (2×40 mL). The organic layer was dried over Na₂SO₄ (anhydrous), filtered and concentrated (DMF was concentrated off at high vacuum pump). The crude residue was flash chromatographed on SiO₂ (25% MeOH/CF₂Cl₂) to furnish a solid that was slurred with Et₂O (6 mL), to furnish 88 mg of (R)-2-acetamido-3-((2′,4′-difluoro-3-(methoxycarbonyl)biphenyl-4-yloxy)carbonyl thio)propanoic acid (off-white solid, yield: 13%).

¹H NMR (CD₃OD, 250 MHz) δ ppm: 8.14 (s, 1H), 7.82 (d, J=8.5 Hz, 1H), 7.59 (m, 1H), 7.36 (d, J=8.5 Hz, 1H), 7.18-7.06 (m, 2H), 4.63 (bs, 1H), 3.94 (s, 3H), 3.66 (d, J=13.1 Hz, 1H), 3.30 (d, J=13.1 Hz, 1H), 2.0 (s, 3H).

EI MS: m/z=454 (M+1).

### Example B-2

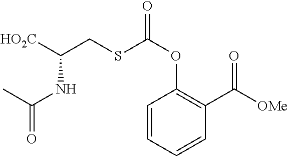 [SMILES: COC(=O)c1ccccc1OC(=O)SC[C@H](NC(C)=O)C(=O)O]

### (R)-2-Acetamido-3-((2-(methoxycarbonyl)phenoxy)carbonylthio)propanoic acid

### Step 1: Methyl 2-hydroxybenzoate

H₂SO₄ (4.0 mL, 74.63 mmol) was added to a solution of salicylic acid (5.0 g, 36.20 mmol) in MeOH (60 mL). The reaction mixture was refluxed for 20 h, allowed to reach r.t., poured into H₂O (100 mL) and extracted with Cl₂Cl₂ (120 mL). The organic layer was dried over Na₂SO₄ (anhydrous), filtered and concentrated. The crude residue was flash chromatographed on SiO₂ (5% EtOAc/hexanes) to furnish 5.44 g of methyl 2-hydroxybenzoate (colourless oil, yield: 98%).

¹H NMR (CDCl₃, 250 MHz) δ ppm: 10.75 (s, 1H), 7.84 (dt, J₁=6.6 Hz, J₂=8.2 Hz, 1H), 7.45 (m, 1H), 6.98 (d, J=8.2 Hz, 1H), 6.87 (m, 1H), 3.95 (s, 3H).

### Step 2: (R)-2-Acetamido-3-((2-(methoxycarbonyl)phenoxy)carbonylthio) propanoic acid

The compound was synthesized from methyl 2-hydroxybenzoate and NAC following the experimental procedure detailed in Method A. It was purified by flash chromatography on SiO₂ (5% MeOH/CH₂Cl₂) to furnish (R)-2-acetamido-3-((2-(methoxycarbonyl) phenoxy)carbonylthio)propanoic acid (white solid, yield: 68%).

¹H NMR (CD₃OD, 250 MHz) δ ppm: 7.98 (dd, J₁=8.0 Hz, J₂=7.7 Hz, 1H), 7.63 (t, J=8.0 Hz, 1H), 7.40 (t, J=7.7 Hz, 1H), 7.22 (d, J=8.2 Hz, 1H), 4.55 (m, 1H), 3.86 (s, 3H), 3.63 (d, J=13.7 Hz, 1H), 3.26 (d, J=13.7 Hz, 1H), 2.0 (s, 3H).

EI MS: m/z=342 (M+1).

### Example B-3

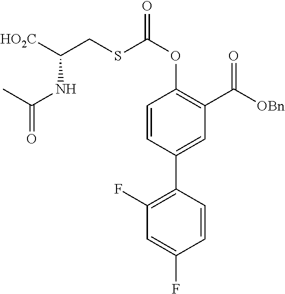 [SMILES: CC(=O)N[C@@H](CSC(=O)Oc1ccc(-c2ccc(F)cc2F)cc1C(=O)OCc1ccccc1)C(=O)O]

### (R)-2-Acetamido-3-((2′,4′-difluoro-3-(benzyloxycarbonyl)biphenyl-4-yloxy)carbonylthio)propanoic acid

### Step 1: Benzyl 2′,4′-difluoro-4-hydroxybiphenyl-3-carboxylate

BnBr (1.35 mL, 11.286 mmol) was added to a solution of diflunisal (2.0 g, 7.99 mmol) in TBAF (10 mL, 1 M solution in THF) and the reaction mixture was stirred at r.t. overnight (18 h). The organic layer was poured into H₂O (15 mL) and extracted with EtOAc (40 mL). It was dried over Na₂SO₄ (anhydrous), filtered and concentrated. The crude residue was flash chromatographed on SiO₂ (hexanes) to furnish 2.09 g of benzyl 2′,4′-difluoro-4-hydroxybiphenyl-3-carboxylate (white solid, yield: 77%).

¹H NMR (CDCl₃, 250 MHz) δ ppm: 10.84 (s, 1H), 8.00 (s, 1H), 7.60 (m, 1H), 7.29-7.47 (6H), 7.06 (d, J=8.4 Hz, 1H), 6.91 (m, 2H), 5.42 (s, 2H).

### Step: 2: (R)-2-Acetamido-3-((2′ 4′-difluoro-3-(benzyloxycarbonyl)biphenyl-4-yloxy)carbonylthio)propanoic acid

The compound was synthesized from benzyl 2′,4′-difluoro-4-hydroxybiphenyl-3-carboxylate and NAC following the experimental procedure detailed in Method A. It was purified by flash chromatography on SiO₂ (7% MeOH/CH₂Cl₂) to furnish (R)-2-acetamido-3-((2′,4′-difluoro-3-(benzyloxycarbonyl)biphenyl-4-yloxy)carbonylthio)propanoic acid (white solid, yield: 9%). ¹H NMR (CD₃OD, 250 MHz) δ ppm: 8.11 (s, 1H), 7.77 (m, 1H), 7.58-7.27 (m, 7H), 7.06 (m, 2H), 5.35 (s, 2H), 4.60 (m, 1H), 3.51 (m, 1H), 3.18 (m, 1H), 1.96 (s, 3H).

EI MS: m/z=530 (M+1).

### Example B-4

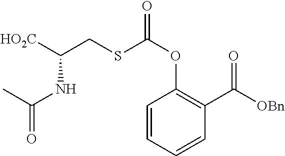 [SMILES: CC(=O)N[C@@H](CSC(=O)Oc1ccccc1C(=O)OCc1ccccc1)C(=O)O]

### (R)-2-Acetamido-3-((2-(benzyloxycarbonyl)phenoxy)carbonylthio)propanoic acid

### Step 1: Benzyl 2-hydroxybenzoate

BnBr (1.75 mL, 14.631 mmol) was added to a solution of salicylic acid (2.0 g, 14.625 mmol) in TBAF (17.5 mL, 1 M solution in THF) and the reaction mixture was stirred at r.t. overnight (16 h). The organic layer was poured into H₂O (100 mL) and extracted with EtOAc (70 mL). It was dried over Na₂SO₄ (anhydrous), filtered and concentrated. The crude residue was flash chromatographed on SiO₂ (2% EtOAc/hexanes) to furnish 2.53 g of benzyl 2-hydroxybenzoate (colourless oil, yield: 76%).

¹H NMR (CDCl₃, 250 MHz) δ ppm: 10.75 (s, 1H), 7.87 (t, J=7.1 Hz, 1H), 7.42 (m, 6H), 6.97 (t, J=7.4 Hz, 1H), 6.86 (c, J=7.1 Hz, 1H), 5.38 (s, 2H).

### Step 2: (R)-2-Acetamido-3-((2-(benzyloxycarbonyl)phenoxy)carbonylthio)propanoic acid

The compound was synthesized from benzyl 2-hydroxybenzoate and NAC following the experimental procedure detailed in Method A. It was purified by flash chromatography on SiO₂ (10% MeOH/CH₂Cl₂) to furnish (R)-2-acetamido-3-((2-(benzyloxycarbonyl)phenoxy) carbonylthio)propanoic acid (white solid, yield: 22%).

¹H NMR (CD₃OD, 250 MHz) □ ppm: 8.00 (d, J=7.4 Hz, 1H), 7.64 (t, J=7.4 Hz, 1H), 7.48-7.28 (m, 6H), 7.22 (d, J=8.8 Hz, 1H), 5.32 (s, 2H), 4.55 (m, 1H), 3.52 (d, J=13.7 Hz, 1H), 3.17 (d, J=13.7 Hz, 1H), 1.96 (s, 3H).

EI MS: m/z=418 (M+1).

### Example B-5

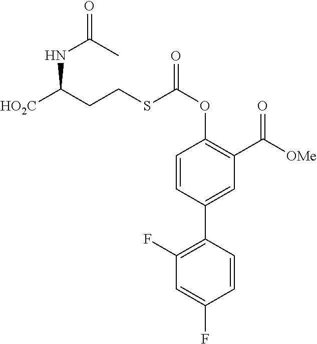 [SMILES: COC(=O)c1cc(-c2ccc(F)cc2F)ccc1OC(=O)SCC[C@H](NC(C)=O)C(=O)O]

### +/−)-2-Acetamido-4((2′,4′-difluoro-3-(methoxycarbonyl)biphenyl-4-yloxy)carbonylthio)butanoic acid (GMC-300

### Step 1: DL-N-Acetylhomocysteine

NaOH (1 M deoxygenated aqueous solution, 100 mL) was dropwise added to DL-N-acetylhomocysteine thiolactone (4.00 g, 25.124 mmol). Addition time: 15 min. The reaction mixture was warmed up to 50° C. and allowed to react for 30 min. The reaction was cooled down to 0° C., acidified with HCl (10% aqueous solution, 30 mL) and the product was extracted with nBuOH (4×70 mL). The organic layer was dried over Na₂SO₄ (anhydrous), filtered and concentrated. The crude residue was slurred with Et₂O/hexanes (1:3, 150 mL) to furnish 4.00 g of DL-N-acetylhomocysteine (white solid, yield: 90%).

¹H NMR (D₂O, 250 MHz) δ ppm: 6.04 (m, 1H), 4.05 (m, 2H), 3.44-3.60 (m, 5H).

### Step 2: (+/−)-2-Acetamido-4-((2′,4′-difluoro-3-(methoxycarbonyl)biphenyl-4-yloxy)carbonylthio)butanoic acid

The compound was synthesized from methyl 2′,4′-difluoro-4-hydroxybiphenyl-3-carboxylate and N-acetylhomocysteine following the experimental procedure detailed in Method A. It was purified by flash chromatography on SiO₂ (0□6% MeOH/CH₂Cl₂) to furnish (+/−)-2-acetamido-4-((2′,4′-difluoro-3-(methoxycarbonyl)biphenyl-4-yloxy)carbonylthio)butanoic acid (off-white solid, yield: 15%).

¹H NMR (CD₃OD, 250 MHz) δ ppm: 8.10 (s, 1H), 7.77 (d, J=8.1 Hz, 1H), 7.56 (m, 1H), 7.32 (d, J=8.1 Hz, 1H), 7.10 (m, 2H), 4.52 (m, 1H), 3.90 (s, 3H), 3.04 (m, 2H), 2.30 (m, 1H), 2.10 (m, 1H), 2.00 (s, 3H).

EI MS: m/z=468 (M+1).

### Example B-6

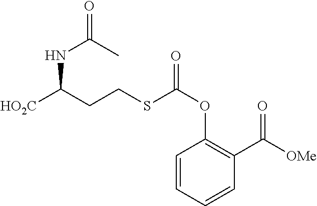 [SMILES: COC(=O)c1ccccc1OC(=O)SCC[C@H](NC(C)=O)C(=O)O]

### (+/−)-2-Acetamido-4-((2-(methoxycarbonyl)phenoxy)carbonylthio) butanoic acid

The compound was synthesized from methyl 2-hydroxybenzoate and N-acetylhomocysteine following the experimental procedure detailed in Method A. It was purified by flash chromatography on SiO₂ (3→10% MeOH/CH₂Cl₂) to furnish (+/−)-2-acetamido-4-((2-(methoxycarbonyl)phenoxy)carbonylthio)butanoic acid (yellow-coloured oil, yield: 13%). ¹H NMR (CDCl₃, 250 MHz) δ ppm: 8.01 (d, J=8.0 Hz, 1H), 7.58 (t, J=7.7 Hz, 1H), 7.35 (t, J=7.7 Hz, 1H), 7.17 (d, J=8.0 Hz, 1H), 6.62 (s, 1H), 4.62 (m, 1H), 3.90 (s, 3H), 3.00 (m, 2H), 2.33 (m, 1H), 2.18 (m, 1H), 2.05 (s, 3H).

EI MS: m/z=356 (M+1).

### Example B-7

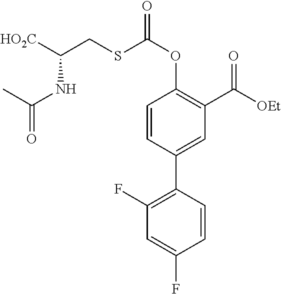 [SMILES: CCOC(=O)c1cc(-c2ccc(F)cc2F)ccc1OC(=O)SC[C@H](NC(C)=O)C(=O)O]

### (R)-2-Acetamido-3-((2′,4′-difluoro-3-(ethoxycarbonyl)biphenyl-4-yloxy)carbonylthio)propanoic acid

### Step 1: Ethyl 2′,4′-difluoro-4-hydroxybiphenyl-3-carboxylate

H₂SO₄ (1 mL, 18.76 mmol) was added to a solution of diflunisal (1.50 g, 5.995 mmol) in EtOH (50 mL). The reaction mixture was refluxed for 2 days, allowed to reach r.t., and diluted with CH₂Cl₂ (200 mL). The organic layer was washed with Na₂CO₃ (1 M aqueous solution, 200 mL). It was dried over Na₂SO₄ (anhydrous), filtered and concentrated to furnish 1.30 g of ethyl 2′,4′-difluoro-4-hydroxybiphenyl-3-carboxylate (white solid, yield: 78%).

¹H NMR (CDCl₃, 250 MHz) δ ppm: 10.92 (s, 1H), 7.97 (m, 1H), 7.59 (dt, J₁=6.6 Hz, J₂=8.5 Hz, 1H), 7.37 (m, 1H), 7.05 (d, J=8.5 Hz, 1H), 6.86-6.99 (m, 2H), 4.44 (c, J=7.1 Hz, 2H), 1.43 (t, J=7.) Hz, 3H).

### Step 2: (R)-2-Acetamido-3-((2′,4′-difluoro-3-(ethoxycarbonyl)biphenyl-4-yloxy) carbonylthio)propanoic acid

The compound was synthesized from ethyl 2′,4′-difluoro-4-hydroxybiphenyl-3-carboxylate and NAC following the experimental procedure detailed in Method A. It was purified by flash chromatography on SiO₂ (3→7% MeOH/CH₂Cl₂) to furnish (R)-2-acetamido-3-((2′,4′-difluoro-3-(ethoxycarbonyl)biphenyl-4-yloxy)carbonylthio)propanoic acid (off-white solid, yield: 28%).

¹H NMR (CD₃OD, 250 MHz) δ ppm: 8.09 (m, 1H), 7.76 (m, 1H), 7.55 (m, 1H), 7.32 (d, J=8.4 Hz, 1H), 7.05-7.16 (m, 2H), 4.58 (m, 1H), 4.35 (c, J=7.0 Hz, 1H), 3.66 (m, 1H), 3.25 (m, 1H), 2.01 (s, 3H), 1.38 (t, J=7.0 Hz, 3H).

EI MS: m/z=468 (M+1).

### Example B-8

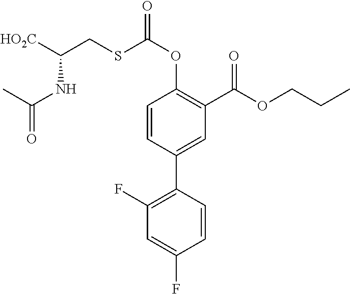 [SMILES: CCCOC(=O)c1cc(-c2ccc(F)cc2F)ccc1OC(=O)SC[C@H](NC(C)=O)C(=O)O]

### R)-2-Acetamido-3-((2′,4′-difluoro-3-(propoxycarbonyl)biphenyl-4-yloxy) carbonylthio)propanoic acid (GMC-316)

### Step 1: Propyl 2′,4′-difluoro-4-hydroxybiphenyl-3-carboxylate

CDI (972 mg, 5.99 mmol) was added to a solution of diflunisal (1.50 g, 5.99 mmol) in DMF (30 mL). The reaction mixture was stirred at 50° C. for 2 h and n-PrOH (1.13 mL, 14.97 mmol) was dropwise added. The reaction mixture was stirred at 50° C. for 3 bond allowed to reach r.t. It was poured into H₂O (50 mL) and extracted with Et₂O (2×50 mL) The organic layer was washed with NaHCO₃ (20 mL, saturated aqueous solution), dried over Na₂SO₄ (anhydrous), filtered and concentrated. The crude residue was flash chromatographed on SiO₂ (2% EtOAc/hexanes) to furnish 1.33 g of propyl 2′,4′-difluoro-4-hydroxybiphenyl-3-carboxylate (yellow-coloured oil, yield: 76%). ¹H NMR (CDCl₃, 250 MHz) δ ppm: 10.92 (s, 1H), 7.98 (m, 1H), 7.59 (dt, J=6.9 Hz, J₂=8.8 Hz, 1H), 7.36 (m, 1H), 7.05 (d, J=8.8 Hz, 1H), 6.86-7.00 (m, 2H), 4.33 (t, J=6.6 Hz, 2H), 1.82 (m, 2H), 1.04 (t, J=7.4 Hz, 3H).

### Step 2: (R)-2-Acetamido-3-((2′,4′-difluoro-3-(propoxycarbonyl)biphenyl-4-yloxy) carbonylthio)propanoic acid

The compound was synthesized from propyl 2′,4′-difluoro-4-hydroxybiphenyl-3-carboxylate and NAC following the experimental procedure detailed in Method A. It was purified by flash chromatography on SiO₂ (3→5% MeOH/CH₂Cl₂) to furnish (R)-2-acetamido-3-((2′,4′-difluoro-3-(propoxycarbonyl)biphenyl-4-yloxy)carbonylthio)propanoic acid (off-white solid, yield: 16%). ¹H NMR (CD₃OD, 250 MHz) δ ppm: 8.10 (m, 1H), 7.77 (dt, J₁=8.6 Hz, J₂=6.5 Hz), 7.55 (m, 1H), 7.32 (d, 1=8.6 Hz, 1H), 7.06-7.15 (m, 2H), 4.56 (m, 1H), 4.26 (t, J=6.5 Hz, 2H), 3.65 (dd, =13.5 Hz, J₂=4.3 Hz, 1H), 3.26 (m, 1H), 2.01 (s, 3H), 1.79 (m, 2H), 1.01 (t, J=7.6 Hz, 3H). EI MS: m/z=480 (M−1).

### Example B-9

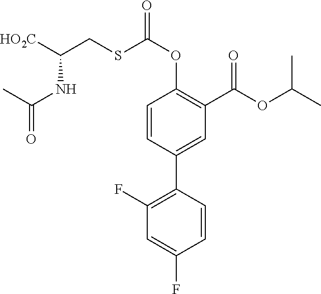 [SMILES: CC(=O)N[C@@H](CSC(=O)Oc1ccc(-c2ccc(F)cc2F)cc1C(=O)OC(C)C)C(=O)O]

### (R)-2-Acetamido-3-((2′,4′-difluoro-3-(isopropoxycarbonyl)biphenyl-4-yloxy) carbonylthio)propanoic acid

### Step 1: Isopropyl 2′,4′-difluoro-4-hydroxybiphenyl-3-carboxylate

CDI (972 mg, 5.99 mmol) was added to a solution of diflunisal (1.50 g, 5.99 mmol) in DMF (30 mL). The reaction mixture was stirred at 50° C. for 2 h and isopropyl alcohol (1.15 mL, 14.97 mmol) was dropwise added. The reaction mixture was stirred at 50° C. for 3 h and allowed to reach r.t. It was poured into H₂O (50 mL) and extracted with Et₂O (2×60 mL). The organic layer was washed with NaHCO₃ (20 mL, saturated aqueous solution), dried over Na₂SO₄ (anhydrous), filtered and concentrated. The crude residue was flash chromatographed on SiO₂ (15% EtOAc/hexanes) to furnish 720 mg of isopropyl 2′,4′-difluoro-4-hydroxybiphenyl-3-carboxylate (white solid, yield: 41%). ¹H NMR (CDCl₃, 250 MHz) 6 ppm: 11.03 (s, 1H), 7.94 (m, 1H), 7.58 (dt, J₁=8.5 Hz, J₂=8.0 Hz, 1H), 7.37 (m, 1H), 7.05 (d, J=8.8 Hz, 1H), 6.93 (m, 2H), 5.31 (m, 1H), 1.41 (s, 3H), 1.39 (s, 3H).

### Step 2: (R)-2-Acetamido-3-((2′,4′-difluoro-3-(isopropoxycarbonyl)biphenyl-4-yloxy)carbonylthio)propanoic acid

The compound was synthesized from isopropyl 2′,4′-difluoro-4-hydroxybiphenyl-3-carboxylate and NAC following the experimental procedure detailed in Method A. It was purified by flash chromatography on SiO₂ (0→10% MeOH/CH₂Cl₂) to give a solid that was slurred with cold hexanes, to furnish (R)-2-acetamido-3-((2′,4′-difluoro-3-(isopropoxycarbonyl)biphenyl-4-yloxy)carbonylthio)propanoic acid (off-white solid, yield: 38%). ¹H NMR (CD₃OD, 250 MHz) δ ppm: 8.07 (m, 1H), 7.76 (m, 1H), 7.55 (m, 1H), 7.32 (d, J=8.5 Hz, 1H), 7.05-7.14 (m, 2H), 5.21 (m, 1H), 4.60 (m, 1H), 3.64 (dd, J₁=13.4 Hz, J₂=4.1 Hz, 1H), 3.26 (m, 1H), 2.00 (s, 3H), 1.37 (s, 3H), 1.35 (s, 3H).

EI MS: m/z=499 (M+18).

### Example B-10

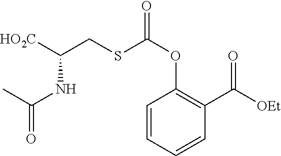 [SMILES: CCOC(=O)c1ccccc1OC(=O)SC[C@H](NC(C)=O)C(=O)O]

### (R)-2-Acetamido-3-((2-(ethoxycarbonyl)phenoxy)carbonylthio)propanoic acid

### Step 1: Ethyl 2-hydroxybenzoate

H₂SO₄ (1.5 mL, 28.14 mmol) was added to a solution of salicylic acid (1.50 g, 10.860 mmol) in EtOH (50 mL). The reaction mixture was refluxed for 2 days, allowed to reach r.t., and it was diluted with CH₂Cl₂ (200 mL). The organic layer was washed with Na₂CO₃ (1 M aqueous solution, 200 mL). It was dried over Na₂SO₄ (anhydrous), filtered and concentrated to furnish 1.26 g of ethyl 2-hydroxybenzoate (yellow-coloured oil, yield: 70%).

¹H NMR (CDCl₃, 250 MHz) δ ppm: 10.89 (s, 1H), 7.84 (dd, J₁=8.5 Hz, J₂=8.0 Hz, 1H), 7.43 (t, J=8.5 Hz, 1H), 6.96 (d, J=8.5 Hz, 1H), 6.86 (t, J=7.3 Hz, 1H), 4.40 (c, J=7.1 Hz, 2H), 1.40 (t, J=7.1 Hz, 3H).

### Step 2: (R)-2-Acetamido-3-((2-(ethoxycarbonyl)phenoxy)carbonylthio)propanoic acid

The compound was synthesized from ethyl 2-hydroxybenzoate and NAC following the experimental procedure detailed in Method A. It was purified by flash chromatography on SiO₂ (0□10% MeOH/CH₂Cl₂) to give a solid that was slurred with cold Et₂O/hexanes (1:10), to furnish (R)-2-acetamido-3-((2-(ethoxycarbonyl)phenoxy)carbonylthio)propanoic acid (off-white solid, yield: 31%). ¹H NMR (CD₃OD, 250 MHz) δ ppm: 7.98 (dd, J₁=8.0 Hz, J₂=7.7 Hz, 1H), 7.63 (dt, Jⱼ=8.0 Hz, J₂=7.4 Hz, 1H), 7.40 (dt, J₁=8.0 Hz, J₂=7.4 Hz, 1H), 7.23 (dd, J₁=8.0 Hz, 0.17=7.7 Hz, 1H), 4.70 (m, 1H), 4.33 (c, J=7.1 Hz, 2H), 3.58 (m, 1H), 3.24 (m, 1H), 2.00 (s, 3H), 1.37 (t, 0.1=7.1 Hz, 3H). EI MS: m/z=356 (M+1).

### Example B-11

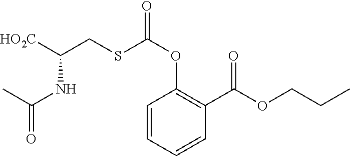 [SMILES: CCCOC(=O)c1ccccc1OC(=O)SC[C@H](NC(C)=O)C(=O)O]

### (R)-2-Acetamido-3-((2-(propoxycarbonyl)phenoxy)carbonylthio)propanoic acid

### Step 1: Propyl 2-hydroxybenzoate

CDI (2.34 g, 14.48 mmol) was added to a solution of salicylic acid (2.00 g, 14.48 mmol) in DMF (40 mL). The reaction mixture was stirred at 50° C. for 4 h and PrOH (2.72 mL, 36.20 mmol) was dropwise added. The reaction mixture was stirred at 50° C. for 16 h and allowed to reach r.t. It was poured into H₂O (20 mL) and extracted with Et₂O (2×40 mL). The organic layer was washed with NaHCO₃ (20 mL, saturated aqueous solution), dried over Na₂SO₄ (anhydrous), filtered and concentrated. The crude residue was flash chromatographed on SiO₂ (5% EtOAc/hexanes) to furnish 2.05 g of propyl 2-hydroxybenzoate (colourless oil, yield: 79%). ¹H NMR (CDCl₃, 250 MHz) δ ppm: 10.85 (s, 1H), 7.85 (dd, =8.5 Hz, J₂=8.0 Hz, 1H), 7.45 (t, J=8.5 Hz, 1H), 6.97 (d, J=8.8 Hz, 1H), 6.88 (t, J=8.0 Hz, 1H), 4.31 (t, 0.1=6.6 Hz, 2H), 1.81 (m, 2H), 1.04 (t, J=7.4 Hz, 3H).

### Step 2: (R)-2-Acetamido-3-((2-(propoxycarbonyl)phenoxy)carbonylthio)propanoic acid

The compound was synthesized from propyl 2-hydroxybenzoate and NAC following the experimental procedure detailed in Method A. It was purified by flash chromatography on SiO₂ (0□6% MeOH/CH₂Cl₂) to give a solid that was slurred with cold hexanes, to furnish (R)-2-acetamido-3-((2-(propoxycarbonyl)phenoxy)carbonylthio)propanoic acid (off-white solid, yield: 17%). ¹H NMR (CD₃OD, 250 MHz) δ ppm: 7.99 (m, 1H), 7.64 (m, 1H), 7.41 (m, 1H), 7.23 (m, 1H), 4.72 (m, 1H), 4.25 (t, J=6.6 Hz, 2H), 3.58 (m, 1H), 3.23 (in, 1H), 2.00 (s, 3H), 1.78 (in, 2H), 1.00 (t, J=7.4 Hz, 3H).

EI MS: m/z=370 (M+1).

### Example B-12

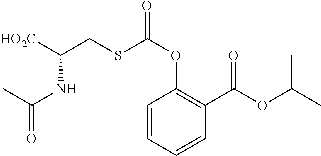 [SMILES: CC(=O)N[C@@H](CSC(=O)Oc1ccccc1C(=O)OC(C)C)C(=O)O]

### (R)-2-Acetamido-3-((2-(isopropoxycarbonyl)phenoxy)carbonylthio)propanoic acid

### Step 1: Isopropyl 2-hydroxybenzoate

CDI (2.34 g, 14.48 mmol) was added to a solution of salicylic acid (2.00 g, 14.48 mmol) in DMF (40 mL). The reaction mixture was stirred at 50° C. for 4 h and isopropyl alcohol (2.80 mL, 36.20 mmol) was dropwise added. The reaction mixture was stirred at 50° C. for 16 h and allowed to reach r.t. It was poured into H₂O (50 mL) and extracted with Et₂O (2×40 mL). The organic layer was washed with NaHCO₃ (20 mL, saturated aqueous solution), dried over Na₂SO₄ (anhydrous), filtered and concentrated. The crude residue was flash chromatographed on SiO₂ (5% EtOAc/hexanes) to furnish 1.395 g of isopropyl 2-hydroxybenzoate (colourless oil, yield: 54%). ¹H NMR (CDCl₃, 250 MHz) δ ppm: 10.94 (s, 1H), 7.84 (d, J=8.0 Hz, 1H), 7.44 (t, J=8.8 Hz, 1H), 6.97 (d, J=8.2 Hz, 1H), 6.87 (t, J=8.2 Hz, 1H), 5.28 (m, 1H), 1.40 (s, 3H), 1.37 (s, 3H).

### Step 2: (R)-2-Acetamido-3-((2-(isopropoxycarbonyl)phenoxy)carbonylthio)propanoic acid

The compound was synthesized from isopropyl 2-hydroxybenzoate and NAC following the experimental procedure detailed in Method A. It was purified by flash chromatography on SiO₂ (3% MeOH/CH₂Cl₂) to furnish (R)-2-acetamido-3-((2-(isopropoxycarbonyl) phenoxy)carbonylthio)propanoic acid (white solid, yield: 23%). ¹H NMR (CD₃OD, 250 MHz) δ ppm: 7.96 (dd, J₁=8.0 Hz, J₂=7.7 Hz, 1H), 7.63 (dt, J₁=7.7 Hz, J₂=7.4, 1H), 7.40 (dt, J₁=7.7 Hz, J?=7.4, 1H), 7.23 (dd, J₁=8.0 Hz, J₂=7.7 Hz, 1H), 5.20 (m, 1H), 4.73 (m, 1H), 3.58 (dd, J=4.7 Hz, 1H), 3.25 (m, 1H), 2.00 (s, 3H), 1.36 (s, 3H), 1.34 (s, 3H). EI MS: m/z=370 (M+1).

### Example B-13

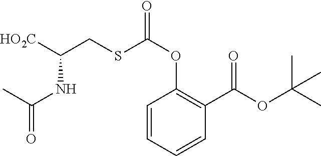 [SMILES: CC(=O)N[C@@H](CSC(=O)Oc1ccccc1C(=O)OC(C)(C)C)C(=O)O]

### (R)-2-Acetamido-3-((2-(tert-butoxycarbonyl)phenoxy)carbonylthio)propanoic acid

### Step 1: tert-Butyl 2-hydroxybenzoate

CDI (2.40 g, 14.79 mmol) was added to a solution of salicylic acid (2.02 g, 14.62 mmol) in DMF (20 mL). The reaction mixture was stirred at 50° C. for 30 min and tert-butyl alcohol (2.80 mL, 29.84 mmol) and DBU (4.4 mL, 29.45 mmol) were dropwise added. The reaction mixture was stirred at 50° C. for 16 h and allowed to reach r.t. It was poured into NaHCO₃ (100 mL, saturated aqueous solution) and extracted with EtOAc (70 mL). The organic layer was dried over Na₂SO₄ (anhydrous), filtered and concentrated. The crude residue was flash chromatographed on SiO₂ (5% EtOAc/hexanes) to furnish 2.089 g of tert-butyl 2-hydroxybenzoate (colourless oil, yield: 73%).

¹H NMR (CDCl₃, 250 MHz) δ ppm: 7.78 (d, J=8.0 Hz, 1H), 7.41 (t, J=7.1 Hz, 1H), 6.95 (d, J=7.1 Hz, 1H), 6.84 (t, J=8.0 Hz, 1H), 1.61 (s, 9H).

### Step 2: (R)-2-Acetamido-3-((2-(tert-butoxycarbonyl)phenoxy)carbonylthio)propanoic acid

The compound was synthesized from tert-butyl 2-hydroxybenzoate and NAC following the experimental procedure detailed in Method A. It was purified by flash chromatography on SiO₂ (5% MeOH/CH₂Cl₂) to furnish (R)-2-acetamido-3-((2-(tert-butoxycarbonyl)phenoxy) carbonylthio)propanoic acid (white solid, yield: 40%). ¹H NMR (CD₃OD, 250 MHz) δ ppm: 7.85 (dd, J=7.7 Hz, 1H), 7.59 (dt, J=8.0 Hz, 1H), 7.38 (dt, J=7.7 Hz, 1H), 7.20 (d, J=8.0 Hz, 1H), 4.68 (m, 1H), 3.59 (dd, J₁=14.3 Hz, J₂=4.9 Hz, 1H), 3.23 (m, 1H), 1.98 (s, 3H), 1.57 (s, 9H).

### Example B-14

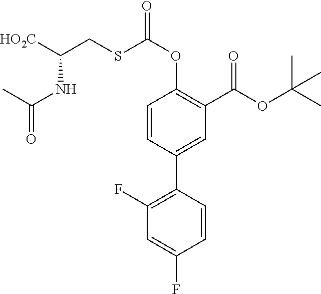 [SMILES: CC(=O)N[C@@H](CSC(=O)Oc1ccc(-c2ccc(F)cc2F)cc1C(=O)OC(C)(C)C)C(=O)O]

### (R)-2-Acetamido-3-((3-(tert-butoxycarbonyl)-2′,4′-difluorobiphenyl-4-yloxy)carbonylthio)propanoic acid

### Step 1: tert-But 2′,4′-difluoro-4-hydroxybiphenyl-3-carboxylate

CDI (1.29 g, 7.99 mmol) was added to a solution of diflunisal (2.02 g, 8.07 mmol) in DMF (20 mL) The reaction mixture was stirred at 50° C. for 30 min and tert-butyl alcohol (1.50 mL, 14.97 mmol) and DBU (2.40 mL, 16.064 mmol) were dropwise added. The reaction mixture was stirred at 50° C. for 20 h and allowed to reach r.t. It was poured into NaHCO₃ (100 mL, saturated aqueous solution) and extracted with EtOAc (100 mL). The organic layer was dried over Na₂SO₄ (anhydrous), filtered and concentrated. The crude residue was flash chromatographed on SiO₂ (5% EtOAc/hexanes) to furnish 1.677 g of tert-butyl 2′,4′-difluoro-4-hydroxybiphenyl-3-carboxylate (colourless oil, yield: 68%). ¹H NMR (CDCl₃, 250 MHz) δ ppm: 11.40 (s, 1H), 7.88 (m, 1H), 7.55 (dt, 1=8.8 Hz, 1H), 7.36 (m, 1H), 6.86-7.04 (m, 3H), 1.62 (s, 9H).

### Step 2: (R)-2-Acetamido-3-((3-(tert-butoxycarbonyl)-2′,4′-difluorobiphenyl-4-yloxy) carbonylthio)propanoic acid

The compound was synthesized from tert-butyl 2′,4′-difluoro-4-hydroxybiphenyl-3-carboxylate and NAC following the experimental procedure detailed in Method A. It was purified by flash chromatography on SiO₂ (3□15% MeOH/CH₂Cl₂) to furnish (R)-2-acetamido-3-((3-(tert-butoxycarbonyl)-2′,4′-difluorobiphenyl-4-yloxy)carbonylthio)propanoic acid (off-white solid, yield: 60%). ¹H NMR (CD₃OD, 250 MHz) δ ppm: 7.96 (m, 1H), 7.72 (d, J=7.9 Hz, 1H), 7.54 (q, J=8.7 Hz, 1H), 7.29 (d, J=8.7 Hz, 1H), 7.09 (m, 2H), 4.61 (m, 1H), 3.64 (dd, J₁=14.3 Hz, J₂=4.7 Hz, 1H), 3.26 (m, 1H), 2.00 (s, 3H), 1.59 (s, 9H).

### Example B-15

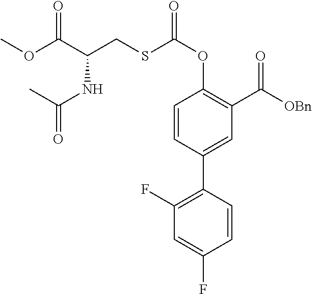 [SMILES: COC(=O)[C@H](CSC(=O)Oc1ccc(-c2ccc(F)cc2F)cc1C(=O)OCc1ccccc1)NC(C)=O]

### (R)-Benzyl 4-((2-acetamido-3-methoxy-3-oxopropylthio)carbonyloxy)-2′,4′-difluorobiphenyl-3-carboxylate

H₂SO₄ (0.020 mL, 0.343 mmol) was added to a solution of (R)-2-acetamido-3-((2′,4′-difluoro-3-(benzyloxycarbonyl)biphenyl-4-yloxy)carbonylthio)propanoic acid (54 mg, 0.102 mmol) in MeOH (10 mL). The reaction mixture was stirred at r.t. for 18 h, poured into H₂O (30 mL) and extracted with CH₂Cl₂ (20 mL) The organic layer was dried over Na₂SO₄ (anhydrous), filtered and concentrated. The crude residue was purified by flash chromatography on SiO₂ (3% MeOH/CH₂Cl₂) to furnish (R)-benzyl 4-((2-acetamido-3-methoxy-3-oxopropylthio)carbonyloxy)-2′,4′-difluorobiphenyl-3-carboxylate (yellow-coloured solid, yield: 82%). ¹H NMR (CD₃OD, 250 MHz) δ ppm: 8.19 (s, 1H), 7.75 (d, J=8.4 Hz, 1H), 7.50-7.37 (m, 5H), 7.30 (m, 1H), 7.03 (d, J=8.4 Hz, 1H), 6.95 (d, J=8.4 Hz, 1H), 6.58 (d, J=7.2 Hz, 1H), 5.41 (s, 2H), 4.96 (m, 1H), 3.80 (s, 3H), 3.51 (dd, J₁=14.4 Hz, J₂=4.5 Hz, 1H), 3.38 (dd, J₁=14.4 Hz, J₂=5.2 Hz, 1H), 2.01 (s, 3H). EI MS: m/z=544 (M+1).

### Example B-16

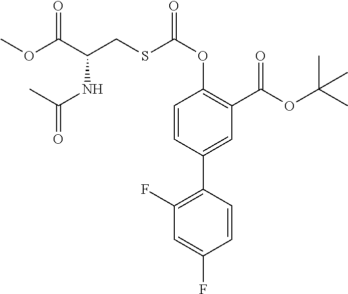 [SMILES: COC(=O)[C@H](CSC(=O)Oc1ccc(-c2ccc(F)cc2F)cc1C(=O)OC(C)(C)C)NC(C)=O]

### (R)-tert-Butyl 4-((2-acetamido-3-methoxy-3-oxopropylthio)carbonyloxy)-2′,4′-difluorobiphenyl-3-carboxylate

H₂SO₄ (0.020 mL, 0.343 mmol) was added to a solution of (R)-2-acetamido-3-((3-(tert-butoxycarbonyl)-2′,4′-difluorobiphenyl-4-yloxy)carbonylthio)propanoic acid (170 mg, 0.343 mmol) in MeOH (5 mL). The reaction mixture was stirred at r.t. for 16 h, poured into H₂O (50 mL) and extracted with CH₂Cl₂ (50 mL). The organic layer was dried over Na₂SO₄ (anhydrous), filtered and concentrated. The crude residue was purified by flash chromatography on SiO₂ (3% MeOH/CH₂Cl₂) to furnish (R)-tert-butyl 4-((2-acetamido-3-methoxy-3-oxopropylthio)carbonyloxy)-2′,4′-difluorobiphenyl-3-carboxylate (yellow-coloured solid, yield: 53%). ¹H NMR (CD₃OD, 250 MHz) δ ppm: 7.99 (s, 1H), 7.65 (in, 1H), 7.40 (m, 1H), 7.19 (in, 1H), 6.89-7.02 (m, 2H), 4.95 (m, 1H), 3.77 (s, 3H), 3.42 (dd, =14.4 Hz, J₂=4.6 Hz, 1H), 3.27 (dd, J₁=14.4 Hz, J₂=5.2 Hz, 1H), 2.02 (s, 3H), 1.59 (s, 9H).

EI MS: m/z=510 (M+1).

### Example B-17

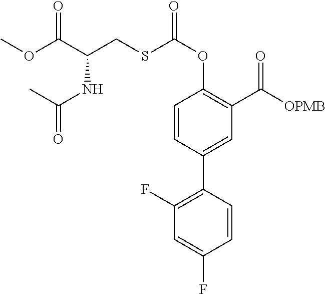

### (R)-2-Acetamido-3-((2′,4′-difluoro-3-((4-methoxybenzyloxy)carbonyl) biphenyl-4-yloxy)carbonylthio)propanoic acid

### Step 1: 4-Methoxybenzyl 2′,4′-difluoro-4-hydroxybiphenyl-3-carboxylate

PMBBr (1.20 mL, 8.325 mmol) was added to a solution of diflunisal (1.50 g, 5.995 mmol) in TBAF (7 mL, 1 M solution in THF) and the mixture was stirred at r.t. overnight (16 h). The reaction mixture was poured into H₂O (100 mL) and extracted with EtOAc (70 mL). It was dried over Na₂SO₄ (anhydrous), filtered and concentrated. The crude residue was flash chromatographed on SiO₂ (5→10% EtOAc/hexanes) to furnish 1.85 g of 4-methoxybenzyl 2′,4′-difluoro-4-hydroxybiphenyl-3-carboxylate (white solid, yield: 83%). ¹H NMR (CDCl₃, 250 MHz) δ ppm: 10.89 (s, 1H), 7.96 (bs, 1H), 7.58 (dt, J₁=8.6 Hz, J₂=1.9 Hz, 1H), 7.39 (d, J=8.9 Hz, 2H), 7.33 (m, 1H), 7.05 (d, J=8.6 Hz, 1H) 6.98-6.83 (m, 4H), 5.34 (s, 2H), 3.81 (s, 3H).

### Step 2: (R)-2-Acetamido-3-((2′,4′-difluoro-3-((4-methoxybenzyloxy)carbonyl)biphenyl-4-yloxy)carbonylthio)propanoic acid

The compound was synthesized from 4-methoxybenzyl 2′,4′-difluoro-4-hydroxybiphenyl-3-carboxylate and NAC following the experimental procedure detailed in Method A. It was purified by flash chromatography on SiO₂ (0→20% MeOH/CH₂Cl₂) to furnish (R)-2-acetamido-3-((2′,4′-difluoro-3-((4-methoxybenzyloxy)carbonyl)biphenyl-4-yloxy)carbonylthio)propanoic acid (off-white solid, yield: 18%). The compound was submitted to next step without characterization.

### Step 3: (R)-4-Methoxybenzyl 4-((2-acetamido-3-methoxy-3-oxopropylthio)carbonyloxy)-2′,4′-difluorobiphenyl-3-carboxylate

H₂SO₄ (0.04 mL, 0.750 mmol) was added to a solution of (R)-2-acetamido-3-((2′,4′-difluoro-3-((4-methoxybenzyloxy)carbonyl)biphenyl-4-yloxy)carbonylthio)propanoic acid (160 mg, 0.286 mmol) in MeOH (25 mL). The reaction mixture was stirred at r.t. for 2 days, poured into H₂O (100 mL) and extracted with CH₂Cl₂ (100 mL). The organic layer was dried over Na₂SO₄ (anhydrous), filtered and concentrated. The crude residue was purified by flash chromatography on SiO₂ (5□80% EtOAc/hexanes) to furnish (R)-4-methoxybenzyl 4-((2-acetamido-3-methoxy-3-oxopropylthio)carbonyloxy)-2′,4′-difluorobiphenyl-3-carboxylate (white solid, yield: 30%). ¹H NMR (CDCl₃, 250 MHz) δ ppm: 8.10 (bs, 1H), 7.68 (dt, J₁=8.5 Hz, J₂=1.6 Hz 1H), 7.37 (d, J=8.5 Hz, 2H), 7.33 (m, 1H), 7.22 (d, J=8.6 Hz, 1H) 7.01-6.85 (m, 4H), 6.56 (d, J=7.6 Hz, 1H), 5.30 (s, 2H), 4.92 (m, 1H), 3.81 (s, 3H), 3.77 (s, 3H), 3.48 (dd, J₁=14.5 Hz, J₂=4.7 Hz, 1H), 3.33 (dd, J₁=14.5 Hz, J₂=5.5 Hz, 1H), 1.99 (s, 3H). EI MS: m/z=574 (M+1).

### Example B-18

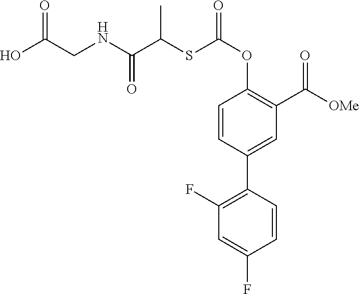 [SMILES: COC(=O)c1cc(-c2ccc(F)cc2F)ccc1OC(=O)SC(C)C(=O)NCC(=O)O]

### 2-(2-((2′,4′-Difluoro-3-(methoxycarbonyl)biphenyl-4-yloxy)carbonylthio)propanamido)acetic acid

The compound was synthesized from methyl 2′,4′-difluoro-4-hydroxybiphenyl-3-carboxylate and N-(2-mercaptopropionyl)glycine following the experimental procedure detailed in Method A, avoiding the addition of Et₃N to the reaction medium. The crude residue was purified by flash chromatography on SiO₂ (2→10% MeOH/CH₂Cl₂) to give a colourless oil that was precipitated by stirring at −78° C. in the presence of hexanes, to furnish 2-(2-((2′,4′-difluoro-3-(methoxycarbonyl)biphenyl-4-yloxy)carbonylthio)propanamido)acetic acid (white solid, yield: 19%). ¹H NMR (CD₃OD, 250 MHz) δ ppm: 8.11 (s, 1H), 7.78 (d, J=8.5 Hz, 1H), 7.55 (m, 1H), 7.34 (d, J=7.34 Hz, 1H), 7.05-7.14 (m, 2H), 4.24 (c, J=6.6 Hz, 1H), 3.85-3.99 (m, 5H), 1.61 (d, J=6.6 Hz, 3H). EI MS: m/z=454 (M+1).

### Example B-19

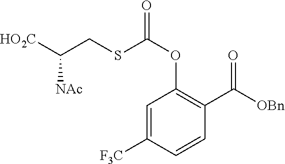

### (R)-2-Acetamido-3-((2-(benzyloxycarbonyl)-5-(trifluoromethyl)phenoxy) carbonylthio)propanoic acid

### Step 1: Benzyl 2-hydroxy-4-(trifluoromethyl)benzoate

BnBr (1.20 mL, 8.325 mmol) was added to a solution of 2-hydroxy-4-(trifluoromethyl)benzoic acid (1.50 g, 7.277 mmol) in TBAF (10 mL, 1 M solution in THF) and the mixture was stirred at r.t. for 2 h. The reaction mixture was poured into NaHCO₃ (saturated aqueous solution, 100 mL) and extracted with EtOAc (100 mL). It was dried over Na₂SO₄ (anhydrous), filtered and concentrated. The crude residue was flash chromatographed on SiO₂ (5→30% EtOAc/hexanes) to furnish 2.18 g of benzyl 2-hydroxy-4-(trifluoromethyl)benzoate (colourless oil, yield: quantitative). ¹H NMR (CDCl₃, 250 MHz) δ ppm: 10.89 (s, 1H), 7.99 (d, J=8.5 Hz, 1H), 7.44 (m, 6H), 7.10 (d, J=8.5 Hz, 1H), 5.42 (s, 2H).

### Step 2: (R)-2-Acetamido-3-((2-(benzyloxycarbonyl)-5-(trifluoromethyl)phenoxy) carbonylthio)propanoic acid

The compound was synthesized from benzyl 2-hydroxy-4-(trifluoromethyl)benzoate and NAC following the experimental procedure detailed in Method A. The crude residue was purified by flash chromatography on SiO₂ (5→20% MeOH/CH₂Cl₂) to give (R)-2-acetamido-3-((2-(benzyloxycarbonyl)-5-(trifluoromethyl)phenoxy)carbonylthio)propanoic acid (white solid, yield: 18%). ¹H NMR (CD₃OD, 250 MHz) δ ppm: 8.17 (d, J=8.2 Hz, 1H), 7.72 (d, J=8.2 Hz, 1H), 7.61 (s, 1H), 7.33-7.48 (m, 5H), 5.36 (s, 2H), 4.54 (m, 1H), 3.54 (dd, =4.1 Hz, J₂=13.7 Hz, 1H), 3.18 (m, 1H), 1.96 (s, 3H). EI MS: m/z=486 (M+1).

### Example C-1

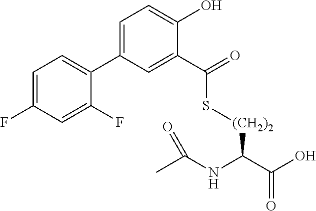

### S)-2-acetamido-4-(2′,4′-difluoro-4-hydroxybiphenylcarbonylthio)butanoic acid (GMC-299

The title compound is prepared using the procedures described in BE 900328 and in International Patent Application Publication no. WO 2010/106082 (see above at Example A-13), each of which is hereby incorporated by reference in its entirety.

### Example C-1b

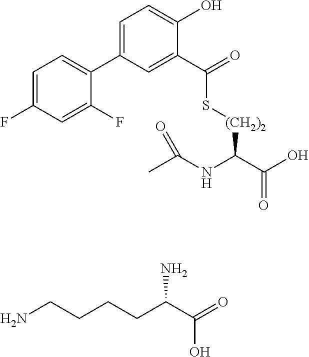

### S)-2-acetamido-4-(2′,4′-difluoro-4-hydroxybiphenylcarbonylthio)butanoic acid L-lysine salt ((3MC-299 lys

The title compound is prepared using the procedures described in BE 900328 and in International Patent Application Publication no. WO 2010/106082 (see above at Example A-13b), each of which is hereby incorporated by reference in its entirety.

It is understood that the examples and embodiments described herein are for illustrative purposes only. Unless clearly excluded by the context, all embodiments disclosed for one aspect of the invention can be combined with embodiments disclosed for other aspects of the invention, in any suitable combination. It will be apparent to those skilled in the art that various modifications and variations can be made to the present invention without departing from the scope of the invention. Thus, it is intended that the present invention cover the modifications and variations of this invention provided they come within the scope of the appended claims and their equivalents. All publications, patents, and patent applications cited herein are hereby incorporated herein by reference for all purposes.

1. A pharmaceutical combination comprising:
(a) an anti-inflammatory agent/anti-oxidant agent conjugate; and
(b) an insulin secretagogue, an insulin sensitizer, an alpha-glucosidase inhibitor, a peptide analog, or a combination thereof.

2. A pharmaceutical combination according to claim 1, wherein the anti-inflammatory agent/anti-oxidant agent conjugate is selected from
(R)-2-acetamido-3-(2-hydroxybenzoylthio)propanoic acid;
(R)-methyl 2-acetamido-3-(2-hydroxybenzoylthio)propanoate;
(R)-ethyl 2-acetamido-3-(2-hydroxybenzoylthio)propanoate;
(R)-2-acetamido-3-(2-acetoxybenzoylthio)propanoic acid;
(R)-methyl 2-acetamido-3-(2-acetoxybenzoylthio)propanoate;
(R)-ethyl 2-acetamido-3-(2-acetoxybenzoylthio)propanoate;
(R)-2-acetamido-3-(2-hydroxy-4-(trifluoromethyl)benzoylthio)propanoic acid;
(R)-methyl 2-acetamido-3-(2-hydroxy-4-(trifluoromethyl)benzoylthio)propanoate;
(R)-ethyl 2-acetamido-3-(2-hydroxy-4-(trifluoromethyl)benzoylthio)propanoate;
(R)-2-acetamido-3-(2-acetoxy-4-(trifluoromethyl)benzoylthio)propanoic acid;
(R)-methyl 2-acetamido-3-(2-acetoxy-4-(trifluoromethyl)benzoylthio)propanoate;
(R)-ethyl 2-acetamido-3-(2-acetoxy-4-(trifluoromethyl)benzoylthio)propanoate;
(R)-2-acetamido-3-(2-acetoxy-4-(trifluoromethyl)benzoylthio)propanoic acid;
(R)-methyl 2-acetamido-3-(2-acetoxy-4-(trifluoromethyl)benzoylthio)propanoate;
(R)-ethyl 2-acetamido-3-(2-acetoxy-4-(trifluoromethyl)benzoylthio)propanoate;
(R)-2-acetamido-3-(2′,4′-difluoro-4-hydroxybiphenylcarbonylthio)propanoic acid (GMC-252);
(R)-methyl 2-acetamido-3-(2′,4′-difluoro-4-hydroxybiphenylcarbonylthio)propanoate;
(R)-ethyl 2-acetamido-3-(2′,4′-difluoro-4-hydroxybiphenylcarbonylthio)propanoate;
(R)-2-acetamido-3-(4-acetoxy-2′,4′-difluorobiphenylcarbonylthio)propanoic acid;
(R)-methyl 2-acetamido-3-(4-acetoxy-2′,4′-difluorobiphenylcarbonylthio)propanoate;
(R)-ethyl 2-acetamido-3-(4-acetoxy-2′,4′-difluorobiphenylcarbonylthio)propanoate;
methyl 2-(5-((R)-1,2-dithiolan-3-yl)pentanoyloxy)benzoate;
tert-butyl 2-(5-((R)-1,2-dithiolan-3-yl)pentanoyloxy)benzoate;
benzyl 2-(5-((R)-1,2-dithiolan-3-yl)pentanoyloxy)benzoate;
(R)-2-acetamido-3-((2′,4′-difluoro-3-(methoxycarbonyl)biphenyl-4-yloxy) carbonylthio)propanoic acid;
(R)-2-acetamido-3-((2-(methoxycarbonyl)phenoxy)carbonylthio)propanoic acid;
(R)-2-acetamido-3-((2′,4′-difluoro-3-(benzyloxycarbonyl)biphenyl-4-yloxy)carbonylthio)propanoic acid;
(R)-2-acetamido-3-((2-(benzyloxycarbonyl)phenoxy)carbonylthio)propanoic acid;
(+/−)-2-acetamido-4-((2′,4′-difluoro-3-(methoxycarbonyl)biphenyl-4-yloxy)carbonylthio)butanoic acid (GMC-300);
(+/−)-2-acetamido-4-((2-(methoxycarbonyl)phenoxy)carbonylthio) butanoic acid (R)-2-acetamido-3-((2′,4′-difluoro-3-(ethoxycarbonyl)biphenyl-4-yloxy)carbonylthio)propanoic acid;
(R)-2-acetamido-3-((2′,4′-difluoro-3-(propoxycarbonyl)biphenyl-4-yloxy) carbonylthio)propanoic acid (GMC-316);
(R)-2-acetamido-3-((2′,4′-difluoro-3-(isopropoxycarbonyl)biphenyl-4-yloxy) carbonylthio)propanoic acid;
(R)-2-acetamido-3-((2-(ethoxycarbonyl)phenoxy)carbonylthio)propanoic acid;
(R)-2-acetamido-3-((2-(propoxycarbonyl)phenoxy)carbonylthio)propanoic acid;
(R)-2-acetamido-3-((2-(isopropoxycarbonyl)phenoxy)carbonylthio)propanoic acid;
(R)-2-acetamido-3-((2-(tert-butoxycarbonyl)phenoxy)carbonylthio)propanoic acid;
(R)-2-acetamido-3-((3-(tert-butoxycarbonyl)-2′,4′-difluorobiphenyl-4-yloxy)carbonylthio)propanoic acid;
(R)-benzyl 4-((2-acetamido-3-methoxy-3-oxopropylthio)carbonyloxy)-2′,4′-difluorobiphenyl-3-carboxylate;
(R)-tert-butyl 4-((2-acetamido-3-methoxy-3-oxopropylthio)carbonyloxy)-2′,4′-difluorobiphenyl-3-carboxylate;
(R)-2-acetamido-3-((2′,4′-difluoro-3-((4-methoxybenzyloxy)carbonyl) biphenyl-4-yloxy)carbonylthio)propanoic acid;
2-(2-((2′,4′-difluoro-3-(methoxycarbonyl)biphenyl-4-yloxy)carbonylthio) propanamido)acetic acid;
(R)-2-acetamido-3-((2-(benzyloxycarbonyl)-5-(trifluoromethyl)phenoxy) carbonylthio)propanoic acid, and
(S)-2-acetamido-4-(2′,4′-difluoro-4-hydroxybiphenylcarbonylthio)butanoic acid (GMC-299), and pharmaceutically acceptable salts thereof.

3. A pharmaceutical combination according to claim 1, wherein the anti-inflammatory agent/anti-oxidant agent conjugate is (R)-2-acetamido-3-(2′,4′-difluoro-4-hydroxybiphenylcarbonylthio)propanoic acid (GMC-252) or a pharmaceutically acceptable salt thereof.

4. A pharmaceutical combination according to claim 1, comprising:
(a) the anti-inflammatory agent/anti-oxidant agent conjugate; and
(b) an insulin secretagogue.

5. A pharmaceutical combination according to claim 1, comprising:
(a) the anti-inflammatory agent/anti-oxidant agent conjugate; and
(b) an insulin sensitizer.

6. A pharmaceutical combination according to claim 5, wherein the insulin sensitizer is metformin.

7. A pharmaceutical combination according to claim 1, comprising:
(a) the anti-inflammatory agent/anti-oxidant agent conjugate; and
(b) an alpha-glucosidase inhibitor.

8. A pharmaceutical combination according to claim 1, comprising:
(a) the anti-inflammatory agent/anti-oxidant agent conjugate; and
(b) a peptide analog.

9. A pharmaceutical combination according to claim 8, wherein the peptide analog is selected from glucagon-like peptide (GLP) analogs and agonists, gastric inhibitory peptide analogs, and amylin analogues.

10. A pharmaceutical combination according to claim 8, wherein the peptide analog is exenatide.

11. A pharmaceutical combination according to claim 1, comprising (R)-2-acetamido-3-(2′,4′-difluoro-4-hydroxybiphenylcarbonylthio)propanoic acid (GMC-252) or a pharmaceutically acceptable salt thereof as the anti-inflammatory agent/anti-oxidant agent, and metformin.

12. A pharmaceutical combination according to claim 1, comprising (R)-2-acetamido-3-(2′,4′-difluoro-4-hydroxybiphenylcarbonylthio)propanoic acid (GMC-252) or a pharmaceutically acceptable salt thereof as the anti-inflammatory agent/anti-oxidant agent, and exenatide.

13. A pharmaceutical composition comprising a pharmaceutical combination according to claim 1 and at least one pharmaceutically acceptable carrier.

14. A method for treating type I diabetes, type II diabetes, Latent Autoimmune Diabetes of Adulthood (LADA), metabolic syndrome, hyperglycemia, or insulin sensitivity in a mammal or human patient in need of such treatment, the method comprising administering to the mammal or human patient a therapeutically effective amount of a pharmaceutical combination according to claim 1.

15. A method for protecting pancreatic β-cells in a mammal or human patient in need of such protection, the method comprising administering to the mammal or human patient a therapeutically effective amount of a pharmaceutical combination according to claim 1.
